(19) United States
(12) Patent Application Publication
(10) Pub. No.: US 2025/0002919 A1
(43) Pub. Date: Jan. 2, 2025

(54) REGENERATION OR REJUVENATION OF TISSUES AND ORGANS

(71) Applicant: The Board of Trustees of the Leland Stanford Junior University, Stanford, CA (US)

(72) Inventors: Helen M. Blau, Stanford, CA (US); Yu Xin Wang, Stanford, CA (US); Daniel Robinson, Stanford, CA (US)

(73) Assignee: The Board of Trustees of the Leland Stanford Junior University, Stanford, CA (US)

(21) Appl. No.: 18/704,002

(22) PCT Filed: Oct. 27, 2022
(86) PCT No.: PCT/US2022/078762

§ 371 Date: Apr. 23, 2024

Related U.S. Application Data
(60) Provisional application No. 63/272,511, filed on Oct. 27, 2021.

(51) Int. Cl. C12N 15/113; A61K 31/713; C12N 9/22; C12N 15/11

(52) U.S. Cl. CPC C12N 15/1137; A61K 31/713; C12N 9/22; C12N 15/111; C12N 2310/20

(57) ABSTRACT

The present disclosure provides compositions and methods based on the use of 15-PGDH and/or PGE2 as a therapeutic target, for example, to regenerate or rejuvenate injured, damaged, aged, or diseased tissue and/or injured, damaged, aged, or diseased organs.

### CROSS-REFERENCE TO RELATED APPLICATIONS

This application claims priority to U.S. Provisional Application No. 63/272,511, filed Oct. 27, 2021, the disclosure of which is hereby incorporated by reference in its entirety for all purposes.

### STATEMENT AS TO RIGHTS TO INVENTIONS MADE UNDER FEDERALLY SPONSORED RESEARCH AND DEVELOPMENT

This invention was made with Government support under contract AG069858 awarded by the National Institutes of Health. The Government has certain rights in the invention.

### BACKGROUND

As an individual ages, quality of life is reduced and mortality is increased. Age-related diseases are a group of diseases that occur more frequently in people as they age, and which can also be caused or accelerated by genetic or other factors, which directly correlate to decreased longevity. Aging and age-related diseases and conditions can have numerous deleterious effects on tissues, leading to an overall loss of function or health of the tissues.

Prostaglandin E2 (PGE2), also known as dinoprostone, has been employed in various clinical settings including to induce labor in women and to augment hematopoietic stem cell transplantation. PGE2 can be used as an anticoagulant and antithrombotic agent. The role of PGE2 as a lipid mediator that can resolve inflammation is also well known. Nonsteroidal anti-inflammatory drugs (NSAIDs), inhibitors of COX-1 and/or COX-2, suppress inflammation by inhibiting prostanoids, mainly via PGE2 biosynthesis. Prostaglandin D2 (PGD2) is a structural isomer of PGE2, with the 9-keto and 11-hydroxy group on PGE2 reversed on PGD2. PGD2 plays a role in a number of biological functions including vasoconstriction, inflammation, the regulation of body temperature during sleep, chemotaxis, and male sexual development. PGE2 and PGD2 are both synthesized from arachidonic acid by cyclooxygenases (COX) and by prostaglandin E synthase enzymes or prostaglandin D synthase enzymes, respectively. Levels of PGE2 and PGD2 are physiologically regulated by the enzyme 15-hydroxyprostaglandin dehydrogenase (15-PGDH), which catalyzes the conversion of the 15-OH group of both PGE2 and PGD2 to a 15-keto group.

### BRIEF SUMMARY

There remains a need in the art for effective treatments for preventing or reversing loss of function in tissues and/or organs, e.g., skeletal muscle and non-skeletal muscle tissues and organs in subjects with diseased or damaged tissues and organs, for example in age-related diseases and disorders and/or in aged subjects and/or where damage to skeletal muscle and non-skeletal muscle tissues and organs occurs to a subject. The present disclosure satisfies this need and provides other advantages as well.

The present disclosure provides compositions and methods for improving the health, function, and/or performance of skeletal and non-skeletal muscle tissues and/or organs in subjects with damaged tissues and/or organs, or in age-related conditions or diseases and/or in aged subjects, in particular by inhibiting 15-PGDH or upregulating PGE2 synthesis in the subjects.

Provided herein are methods, systems and compositions for regenerating and/or rejuvenating a function of an injured, damaged, aged, or diseased tissue or an injured, damaged, aged, or diseased organ in an individual, the method comprising: administering to the individual a 15-hydroxyprostaglandin dehydrogenase (15-PGDH) inhibitor and/or a prostaglandin E2 (PGE2) activator in an amount effective to inhibit 15-PGDH expression or activity and/or increase PGE2 synthesis or activity in the individual, thereby regenerating and/or rejuvenating the function of the injured, damaged, aged, or diseased tissue or the injured, damaged, aged, or diseased organ in the individual, wherein the 15-PGDH inhibitor and/or PGE2 activator is selected from the group consisting of: a nucleic acid molecule or polypeptide capable of inhibiting 15-PGDH expression, a nucleic acid molecule or polypeptide capable of enhancing PGE2 synthesis, a gene editing system capable of inhibiting 15-PGDH expression, a gene editing system capable of enhancing PGE2 synthesis, an epigenetic modification capable of inhibiting 15-PGDH expression, an epigenetic modification capable of enhancing PGE2 synthesis, and combinations thereof. In some instances, after the administering, the function of the injured, damaged, aged, or diseased tissue or the injured, damaged, aged, or diseased organ is regenerated and/or rejuvenated relative to a function of the injured, damaged, aged, or diseased tissue or the injured, damaged, aged, or diseased organ prior to the administering the 15-PGDH inhibitor and/or PGE2 activator. In yet other instances, after the administering, the function of the injured, damaged, aged, or diseased tissue or the injured, damaged, aged, or diseased organ is regenerated and/or rejuvenated by at least about 5% relative to a function of the injured, damaged, aged, or diseased tissue or the injured, damaged, aged, or diseased organ prior to the administering the 15-PGDH inhibitor and/or PGE2 activator. In still other instances, after the administering, the function of the injured, damaged, aged, or diseased tissue or the injured, damaged, aged, or diseased organ is regenerated and/or rejuvenated to a level substantially similar to a level of a function of a young tissue or a young organ.

In still other after the administering, the function of the injured, damaged, aged, or diseased tissue or the injured, damaged, aged, or diseased organ is regenerated and/or rejuvenated to a level within at least about 25% of a level of a function of a young tissue or a young organ. In some cases, after the administering, a level of prostaglandin E2 (PGE2) in the injured, damaged, aged, or diseased tissue or the injured, damaged, aged, or diseased organ is increased relative to a level of PGE2 in the injured, damaged, aged, or diseased tissue or the injured, damaged, aged, or diseased organ prior to the administering. In other cases, after the administering, a level of PGE2 in the injured, damaged, aged, or diseased tissue or the injured, damaged, aged, or diseased organ is increased by at least about 5% relative to a level of PGE2 in the injured, damaged, aged, or diseased tissue or the injured, damaged, aged, or diseased organ prior to the administering. In still other cases, after the administering, a level of PGE2 in the injured, damaged, aged, or diseased tissue or the injured, damaged, aged, or diseased organ is increased to a level substantially similar to a level of PGE2 present in a young tissue or a young organ. In still other embodiments, after the administering, a level of PGE2 in the injured, damaged, aged, or diseased tissue or the injured, damaged, aged, or diseased organ is increased to a level within at least about 25% of a level of PGE2 present in a young tissue or a young organ.

In some instances, the administering of the compositions disclosed herein increases systemic levels of PGE2 in the individual. In yet other instances, the administering results in a regeneration or rejuvenation of serum cytokines to levels substantially similar to serum cytokine levels found in a young individual. In still other cases, the serum cytokines are selected from the group consisting of: interleukin-10 (IL10), interleukin-6 (IL6), betacellulin (BTC), granulocyte-macrophage colony-stimulating factor (GM-CSF), interleukin-13 (IL13), tumor necrosis factor alpha (TNF-α), interleukin-1 beta (IL1b), interleukin-22 (IL22), and any combination thereof. In yet other instances, the administering results in: a decrease in serum IL10 levels relative to serum IL10 levels prior to the administering, a decrease in serum IL6 levels relative to serum IL6 levels prior to the administering, a decrease in serum BTC levels relative to serum BTC levels prior to the administering, a decrease in scrum GM-CSF levels relative to serum GM-CSF levels prior to the administering, a decrease in serum IL13 levels relative to serum IL13 levels prior to the administering, an increase in serum TNF-alpha levels relative to serum TNF-alpha levels prior to the administering, an increase in serum IL1 beta levels relative to serum IL1 beta levels prior to the administering, an increase in serum IL22 levels relative to serum IL22 levels prior to the administering, or any combination thereof.

In some instances, the injured, damaged, aged, or diseased tissue or the injured, damaged, aged, or diseased organ is selected from the group consisting of: skeletal muscle tissue, epidermal tissue, epithelial tissue, vascular tissue, cardiac muscle, brain, bone, cartilage, sensory organs, kidney, thyroid, lung, smooth muscle, brown fat, spleen, liver, heart, small intestine, colon, skin, ovaries and other reproductive tissues, hair, dental tissue, blood, cochlea, and any combination thereof. In still other cases, the individual has one or more biomarkers of aging, tissue or organ injury, tissue or organ damage, or tissue or organ disease. In some instances, the one or more biomarkers of aging, tissue or organ injury, tissue or organ damage, or tissue or organ disease is selected from the group consisting of: an increase in 15-PGDH levels relative to a young individual, a decrease in PGE2 levels relative to a young individual, an increase in a PGE2 metabolite relative to a young individual, an increase or a greater accumulation of senescent cells relative to a young individual, an increase in expression of one or more atrogenes relative to a young individual, a decrease in mitochondria biogenesis and/or function relative to a young individual, and an increase in transforming growth factor pathway signaling relative to a young individual.

In some instances, the 15-PGDH inhibitor and/or PGE2 activator administered to the individual in the methods and systems disclosed herein is the nucleic acid molecule capable of inhibiting 15-PGDH or the nucleic acid molecule capable of enhancing PGE2 synthesis. In some cases, the nucleic acid molecule capable of inhibiting 15-PGDH or the nucleic acid molecule capable of enhancing PGE2 synthesis is selected from the group consisting of: a peptide nucleic acid (PNA), an aptamer, an antisense oligonucleotide, a morpholino oligomer, microRNA, siRNA, shRNA, and combinations thereof. In other instances, the 15-PGDH inhibitor and/or PGE2 activator is the gene editing system capable of inhibiting 15-PGDH expression or the gene editing system capable of enhancing PGE2 synthesis. In some cases, the gene editing system capable of inhibiting 15-PGDH expression or the gene editing system capable of enhancing PGE2 synthesis is selected from the group consisting of: a CRISPR-Cas system, a zinc-finger nuclease system, a transcription activator-like effector nuclease (TALEN) system, and combinations thereof. In still other instances, the gene editing system capable of inhibiting 15-PGDH expression or the gene editing system capable of enhancing PGE2 synthesis comprises the CRISPR-Cas system. In some cases, the CRISPR-Cas system comprises a Cas endonuclease coupled to a guide RNA (gRNA) targeting at least a portion of a polynucleotide sequence encoding 15-PGDH and/or a prostaglandin synthase. In still other cases, the Cas endonuclease is Cas9 endonuclease.

In still other instances, the method comprises using the gene editing system capable of inhibiting 15-PGDH expression to insert a silencer sequence near a polynucleotide sequence encoding 15-PGDH, thereby inhibiting 15-PGDH expression. In still other instances, the method comprises using the gene editing system capable of enhancing PGE2 synthesis to insert an enhancer sequence near a polynucleotide sequence encoding a prostaglandin synthase, thereby enhancing PGE2 synthesis. In yet other instances, the administering results in inhibition of transcription factors that negatively regulate the enhancer or promoter of the HPGD gene. In yet other instances, the administering results in upregulation of transcription factors that enhance an enhancer or promoter of a prostaglandin synthase gene. In still other instances, the prostaglandin synthase gene is selected from the group consisting of: PTGES1, PTGES2, COX1, COX2, and combinations thereof. In yet other instances, the 15-PGDH inhibitor and/or PGE2 activator is the polypeptide capable of inhibiting 15-PGDH expression or the polypeptide capable of enhancing PGE2 synthesis. In still other instances, the polypeptide capable of inhibiting 15-PGDH expression or the polypeptide capable of enhancing PGE2 synthesis is selected from the group consisting of: a peptide, an antibody, a nanobody, and combinations thereof.

In some cases, the individual administered the compositions disclosed herein is a human. In some instances, the 15-PGDH inhibitor reduces or blocks 15-PGDH expression. In other cases, the 15-PGDH inhibitor reduces or blocks enzymatic activity of 15-PGDH. In other instances, the PGE2 activator increases expression of a prostaglandin synthase. In other instances, the PGE2 activator increases expression of an activating factor of a prostaglandin synthase. In still other cases, the administering results in decreased levels of a PGE2 metabolite in the injured, damaged, aged, or diseased tissue or the injured, damaged, aged, or diseased organ relative to the injured, damaged, aged, or diseased tissue or the injured, damaged, aged, or diseased organ prior to the administering of the 15-PGDH inhibitor and/or PGE2 activator and/or to a level that is substantially similar to a level present in young tissue or a young organ. In yet other instances, the PGE2 metabolite is selected from the group consisting of: 15-keto PGE2, 13,14-dihydro-15-keto PGE2, and both.

In one aspect, a method of regenerating and/or rejuvenating an injured, damaged, aged, or diseased (e.g., skeletal muscle and/or non-skeletal muscle) tissue in a subject is provided, the method comprising: administering to the subject an amount of a 15-PGDH-inhibitor and/or PGE2 activator effective to inhibit 15-PGDH activity and/or increase PGE2 synthesis and/or increase PGE2 activity and/or reduce 15-PGDH levels and/or increase PGE2 levels in the subject, thereby regenerating and/or rejuvenating the injured, damaged, aged, or diseased (e.g., skeletal muscle and/or non-skeletal muscle) tissue. In some cases, the administering increases a level of PGE2 in the injured, damaged, aged, or diseased (e.g., skeletal muscle and/or non-skeletal muscle) tissue of the subject. In some cases, a level of PGE2 in the injured, damaged, aged, or diseased (e.g., skeletal muscle and/or non-skeletal muscle) tissue is increased relative to the injured, damaged, aged, or diseased (e.g., skeletal muscle and/or non-skeletal muscle) tissue prior to the administering of the 15-PGDH inhibitor and/or PGE2 activator. In some cases, a level of PGE2 in the injured, damaged, aged, or diseased (e.g., skeletal muscle and/or non-skeletal muscle) tissue is increased by at least 5% or 10% relative to the injured, damaged, aged, or diseased (e.g., skeletal and/or non-skeletal muscle) tissue prior to the administering of the 15-PGDH inhibitor and/or PGE2 activator. In some cases, a level of PGE2 in the injured, damaged, aged, or diseased (e.g., skeletal muscle and/or non-skeletal muscle) tissue is increased to a level substantially similar to a level present in young (e.g., skeletal muscle and/or non-skeletal muscle) tissue. In some cases, a level of PGE2 in the injured, damaged, aged, or diseased (e.g., skeletal muscle and/or non-skeletal muscle) tissue is increased to a level within 25% or within 50% of a level present in young (e.g., skeletal muscle and/or non-skeletal muscle) tissue. In some cases, the injured, damaged, aged, or diseased (e.g., skeletal muscle and/or non-skeletal muscle) tissue is selected from the group consisting of: skeletal muscle tissue, epidermal tissue, epithelial tissue, vascular tissue, cardiac muscle, brain, bone, cartilage, sensory organs, kidney, thyroid, lung, smooth muscle, brown fat, spleen, liver, heart, small intestine, colon, skin, ovaries and other reproductive tissues, hair, dental tissue, blood, cochlea, and any combination thereof.

In some cases, the subject has one or more biomarkers of aging. In some cases, the one or more biomarkers of aging is selected from the group consisting of: an increase in 15-PGDH levels relative to young (e.g., skeletal muscle and/or non-skeletal muscle) tissue, a decrease in PGE2 levels relative to young (e.g., skeletal muscle and/or non-skeletal muscle) tissue, an increase in a PGE2 metabolite relative to young (e.g., skeletal muscle and/or non-skeletal muscle) tissue, an increase or a greater accumulation of senescent cells relative to young (e.g., skeletal muscle and/or non-skeletal muscle) tissue, an increase in expression of one or more atrogenes relative to young (e.g., skeletal muscle and/or non-skeletal muscle) tissue, a decrease in mitochondria biogenesis and/or function relative to young (e.g., skeletal muscle and/or non-skeletal muscle) tissue, and an increase in transforming growth factor pathway signaling relative to young (e.g., skeletal muscle and/or non-skeletal muscle) tissue. In some cases, the aged (e.g., skeletal muscle and/or non-skeletal muscle) tissue has an increased accumulation of senescent cells relative to young (e.g., skeletal muscle and/or non-skeletal muscle) tissue. In some cases, the senescent cells express one or more senescent markers. In some cases, the senescent cells have an increased level of one or more senescent markers relative to non-senescent cells. In some cases, the one or more senescent markers is selected from the group consisting of: p15Ink4b, p16Ink4a, p19Arf, p21, Mmp13, II1a, II1b, and I16. In some cases, the senescent cells are macrophages. In some cases, the method further comprises administering a senolytic agent to the aged (e.g., skeletal muscle and/or non-skeletal muscle) tissue. In some cases, the senolytic agent is selected from the group consisting of: a Bcl2 inhibitor, a pan-tyrosine kinase inhibitor, a combination therapy of dasatinib and quercetin, a flavonoid, a peptide that interferes with the FOXO4-p53 interaction, a selective targeting system of senescent cells using galactooligosaccharide-coated nanoparticles, an HSP90 inhibitor, and combinations thereof.

In some cases, the 15-PGDH inhibitor and/or PGE2 activator is selected from the group consisting of: an antibody, a nanobody, a peptide, a nucleic acid molecule and/or a gene editing system that inhibits 15-PGDH expression or activity and/or enhances PGE2 synthesis or activity. In some cases, the 15-PGDH inhibitor and/or PGE2 activator is selected from the group consisting of peptide nucleic acid (PNA), an aptamer, an antisense oligonucleotide, microRNA, siRNA, and shRNA. In some cases, the 15-PGDH inhibitor and/or PGE2 activator comprises nucleic acid molecules and proteins that can be used to target certain genes involved in a disease or a condition, for example Clustered Regularly Interspaced Short Palindromic Repeats (CRISPR)-associated (Cas) protein-mediated gene editing (CRISPR-Cas), for example CRISPR-Cas9 mediated gene editing, transcription activator-like effector nuclease technology (TALEN) or zinc finger nuclease technology. In yet other cases, the 15-PGDH inhibitor and/or PGE2 activator comprises an epigenetic modification of the genomic structure associated with expression of 15-PGDH and/or synthesis of PGE2. In some cases, the epigenetic modification includes methylation, histone modification and microRNA-mediated genetic silencing.

In some cases, the subject or individual is a human. In some cases, the subject is at least 30 years of age. In some cases, the 15-PGDH inhibitor and/or PGE2 activator reduces or blocks 15-PGDH expression. In some cases, the 15-PGDH inhibitor and/or PGE2 activator reduces or blocks enzymatic activity of 15-PGDH. In some cases, a function of the injured, damaged, aged, or diseased (e.g., skeletal muscle and/or non-skeletal muscle) is enhanced relative to the function of the injured, damaged, aged, or diseased (e.g., skeletal muscle and/or non-skeletal muscle) prior to the administering of the 15-PGDH inhibitor and/or PGE2 activator. In some cases, a function of the injured, damaged, aged, or diseased (e.g., skeletal muscle and/or non-skeletal muscle) tissue is enhanced by at least 5% or 10% relative to the function of the injured, damaged, aged, or diseased (e.g., skeletal muscle and/or non-skeletal muscle) tissue prior to the administering of the 15-PGDH inhibitor and/or PGE2 activator. In some cases, a function of the injured, damaged, aged, or diseased (e.g., skeletal muscle and/or non-skeletal muscle) tissue is enhanced to a level that is substantially similar to a level present in young (e.g., skeletal muscle and/or non-skeletal muscle) tissue. In some cases, a function of the injured, damaged, aged, or diseased (e.g., skeletal muscle and/or non-skeletal muscle) tissue is enhanced to a level that is within 25% or within 50% of a level present in young (e.g., skeletal muscle and/or non-skeletal muscle) tissue. In some cases, the function comprises increased protein synthesis, increased cell proliferation, increased cell survival, decreased protein degradation, or any combination thereof. In some cases, the method results in decreased levels of a PGE2 metabolite in the injured, damaged, aged, or diseased (e.g., skeletal muscle and/or non-skeletal muscle) tissue relative to the injured, damaged, aged, or diseased (e.g., skeletal muscle and/or non-skeletal muscle) tissue prior to the administering of the 15-PGDH inhibitor and/or PGE2 activator and/or to a level that is substantially similar to a level present in young (e.g., skeletal muscle and/or non-skeletal muscle) tissue. In some cases, the PGE2 metabolite is selected from the group consisting of: 15-keto PGE2 and 13,14-dihydro-15-keto PGE2.

In another aspect, a method of enhancing a function of a (e.g., skeletal muscle and/or non-skeletal muscle) tissue in a subject is provided, the method comprising: administering to the subject a 15-PGDH inhibitor and/or PGE2 activator in an amount effective to inhibit 15-PGDH activity and/or increase PGE2 synthesis and/or increase PGE2 activity and/or reduce 15-PGDH levels and/or increase PGE2 levels in the (e.g., skeletal muscle and/or non-skeletal muscle) tissue, thereby enhancing a function of the (e.g., skeletal muscle and/or non-skeletal muscle tissue) in the subject. In some cases, the function is enhanced relative to the (e.g., skeletal muscle and/or non-skeletal muscle) tissue prior to the administering of the 15-PGDH inhibitor and/or PGE2 activator. In some cases, the function is an increase in protein synthesis, an increase in cell proliferation, an increase in cell survival, a decrease in protein degradation, or any combination thereof. In some cases, the subject is less than 30 years of age. In some cases, the subject is greater than 30 years of age.

In another aspect, the present disclosure provides a method for increasing the function of a (e.g., skeletal muscle and/or non-skeletal muscle) tissue in a subject with an age-related disorder, the method comprising administering to the subject a therapeutically effective amount of a 15-hydroxyprostaglandin dehydrogenase (15-PGDH) inhibitor, wherein the administration of the 15-PGDH inhibitor and/or PGE2 activator increases or restores the level of PGE2 and/or PGD2 in the (e.g., skeletal muscle and/or non-skeletal muscle) tissue in the subject.

In some embodiments, the subject is suspected of having or at risk for developing the condition or disease associated with muscle damage, injury, or atrophy. In some cases, the condition or disease associated with muscle damage, injury or atrophy is selected from the group consisting of acute muscle injury or trauma, soft, tissue hand injury, spinal muscular atrophy (SMA), Duchenne muscular dystrophy (DMD). Becker muscular dystrophy, limb girdle muscular dystrophy, amyotrophic lateral sclerosis (ALS), distal muscular dystrophy (DD), inherited myopathies, myotonic muscular dystrophy (MOD), mitochondrial myopathies, myotubular myopathy (MM), myasthenia gravis (MG), congestive heart failure, periodic paralysis, polymyositis, rhabdomyolysis, dermatomyositis, cancer cachexia, AIDS cachexia, cardiac cachexia, stress induced urinary incontinence, and sarcopenia. In some embodiments of the method, the age-related disorder is selected from the group consisting of cardiovascular disease, chronic respiratory disease, nutritional disease, kidney disease, gastrointestinal or digestive disease, neurological disorder, sensory disorder, hearing disorder, skin or subcutaneous disease, cerebrovascular disease, osteoporosis, osteoarthritis, premature aging disease, and combinations thereof. In some embodiments, the cardiovascular disease is atrial fibrillation, stroke, ischemic heart disease, cardiomyopathy, endocarditis, intracerebral hemorrhage, hypertension, or a combination thereof. In some embodiments, the chronic respiratory disease is chronic obstructive pulmonary disease, asbestosis, silicosis, or a combination thereof. In some embodiments, the nutritional disease is trachoma, diarrheal disease, encephalitis, or a combination thereof. In some embodiments, the kidney disease is a chronic kidney disease. In some embodiments, the gastrointestinal or digestive disease is NASH, pancreatitis, ulcer, intestinal obstruction, or a combination thereof. In some embodiments, the neurological disorder is Alzheimer's disease, dementia, Parkinson's disease, or a combination thereof. In some embodiments, the sensory disorder is hearing loss, vision loss, loss of sense of smell or sense of taste, macular degeneration, retinosa pigmentosa, glaucoma, or a combination thereof. In some embodiments, the skin or subcutaneous disease is cellulitis, ulcer, fungal skin disease, pyoderma, or a combination thereof. In some embodiments, the premature aging disease is Osteogenesis imperfecta, Bloom syndrome, Cockayne Syndrome, Hutchinson-Gilford Progeria Syndrome, Mandibuloacral Dysplasia, Progeria, Progeroid Syndrome, Rothmund-Thomson Syndrome, Seip Syndrome, Werner Syndrome, Down Syndrome, Acrogeria, Rothmund-Thomson syndrome, an immunodeficiency leading to a premature aging syndrome such as Ataxia telangiectasia, or an infectious disease leading to premature aging such as HIV.

In some embodiments of the method, the subject is a human. In some embodiments, the method further comprises a step in which the human is selected for treatment with the 15-PGDH inhibitor and/or PGE2 activator based on a diagnosis of the age-related disorder. In some embodiments, the (e.g., skeletal muscle and/or non-skeletal muscle) tissue is selected from the group consisting of epidermal tissue, epithelial tissue, vascular tissue, cardiac muscle, brain, bone, cartilage, sensory organs, kidney, thyroid, lung, smooth muscle, brown fat, spleen, liver, heart, brain, small intestine, colon, skin, ovaries and other reproductive tissues, hair, dental tissues, cochlea, oligodendrocytes, and combinations thereof.

In some embodiments of the method, the 15-PGDH inhibitor and/or PGE2 activator inactivates 15-PGDH or blocks 15-PGDH activity. In some embodiments, the 15-PGDH inhibitor and/or PGE2 activator reduces or blocks the enzymatic activity of 15-PGDH. In some embodiments, the 15-PGDH inhibitor and/or PGE2 activator is a blocking antibody, nanobody, or peptide. In some embodiments, the 15-PGDH inhibitor and/or PGE2 activator reduces or blocks 15-PGDH expression. In some embodiments, the 15-PGDH inhibitor and/or PGE2 activator is a peptide nucleic acid (PNA), an aptamer, antisense oligonucleotide, microRNA, siRNA, or shRNA.

In some embodiments of the method, the administration of the 15-PGDH inhibitor and/or PGE2 activator increases or restores the level of PGE2 in the (e.g., skeletal muscle and/or non-skeletal muscle) tissue in the subject. In some embodiments, the therapeutically effective amount of the 15-PGDH inhibitor and/or PGE2 activator decreases PGE2 and/or PGD2 metabolite levels in the (e.g., skeletal muscle and/or non-skeletal muscle) tissue of the subject. In some embodiments, the PGE2 metabolite is 15-keto-PGE2 or 13,14-dihydro-15-keto-PGE2 (PGEM). In some embodiments, the PGD2 metabolite is 15-keto-PGD2 or 13,14-dihydro-15-keto-PGD2. In some embodiments, the therapeutically effective amount of the 15-PGDH inhibitor and/or PGE2 activator increases protein synthesis, increases cell proliferation, increases cell survival, lengthens telomeres, and/or decreases protein degradation, in the (e.g., skeletal muscle and/or non-skeletal muscle) tissue of the subject. In some embodiments, administering the 15-PGDH inhibitor and/or PGE2 activator comprises systemic administration. In some embodiments, administering the 15-PGDH inhibitor and/or PGE2 activator comprises local administration. In some embodiments, the (e.g., skeletal muscle and/or non-skeletal muscle) tissue has an increased accumulation of senescent cells relative to young (e.g., skeletal muscle and/or non-skeletal muscle) tissue. In some embodiments, the method further comprises administering a senolytic agent to the subject. In some embodiments, the senolytic agent is selected from the group consisting of a Bcl2 inhibitor, a pan-tyrosine kinase inhibitor, a flavonoid, a peptide that interferes with the FOXO4-p53 interaction, a selective targeting system of senescent cells using galactooligosaccharide-coated nanoparticles, an HSP90 inhibitor, and combinations thereof.

In yet other embodiments, the methods, compositions and systems disclosed herein further comprise one or more additional therapeutic agents, e.g., agents for administering in combination with the agent that antagonizes the expression or activity of 15-PGDH or increases the expression or activity of PGE-2. In some embodiments, the combination therapy further comprises an inhibitor of 15-PGDH expression or activity and/or an enhancer of PGE-2 expression or activity and a composition that further treats the injured, damaged, aged, or diseased tissue or organ, including, for example, gene editing or gene replacement to correct dysfunctional nucleic acid or protein expression associated with the injured, damaged, aged, or diseased tissue or organ. The combination therapeutic agent may be, for example, a small molecule inhibitor of 15-PGDH and/or an enhancer of PGE2. Replacement of the defective SMN1 gene, for example, in spinal muscular atrophy patients is also contemplated using, for example, gene editing (e.g., CRISPR-Cas mediated correction of the mutant SMN1 gene), gene therapy (e.g., AAV gene therapy to add or replace defective SMN1 gene), nucleic acid therapy (e.g., splice therapy to produce a functional transcript) or peptide therapy in addition to the agent that antagonizes the expression or activity of 15-PGDH or increases the expression of PGE-2.

Other objects, features, and advantages of the present disclosure will be apparent to one of skill in the art from the following detailed description.

### DETAILED DESCRIPTION

### 1. Introduction

The present disclosure is based, in part, on the discovery that the PGE2 degrading enzyme, 15-PGDH, or its transcript, is elevated in a range of aging tissues and/or organs, in particular, skeletal muscle and/or non-skeletal muscle tissues and/or organs. As such, 15-PGDH proteins or transcripts can be used as a biomarker for aging in skeletal muscle and/or non-skeletal muscle tissues and/or organs, e.g., in subjects with an age-related disorder or disease and/or in aged subjects. In addition, 15-PGDH can be inhibited in order to reverse or slow aging and aging-related processes in skeletal muscle and/or non-skeletal muscle tissues and/or organs, thereby ameliorating their function. Without being bound by the following theory, it is believed that elevated 15-PGDH levels in skeletal muscle and/or non-skeletal muscle tissues and/or organs in subjects with age-related conditions or diseases and/or in aged subjects, e.g., in the colon, brain, skin, spleen, or liver, leads to PGE2 and/or PGD2 degradation in these tissues and/or organs and thus to lower levels of PGE2 and/or PGD2 and of PGE2 and/or PGD2 signaling, which has deleterious effects on tissue function that are manifested in aging. The present disclosure therefore provides compositions and methods based on the use of 15-PGDH activity as a therapeutic target in skeletal muscle and/or non-skeletal muscle tissues and/or organs in subjects with age-related diseases or conditions and/or in aged subjects. Inhibiting 15-PGDH and/or upregulating PGE2 synthesis in these tissues and/or organs may restore or increase PGE2 and/or PGD2 levels in the tissues and/or organs and may ameliorate their function, health, and/or physiological activity. Reducing 15-PGDH can thus lead to improved quality of life and outcomes for age-related diseases or disorders.

A non-limiting list of skeletal muscle and/or non-skeletal muscle tissues and/or organs that can be treated using the present methods and compositions include, for example, skeletal muscle, epidermal tissue, vascular tissue, cardiac muscle, brain, bone, cartilage, smooth muscle, brown fat, spleen, liver, and the like. In some embodiments, the subject has, is suspected of having, or is at risk for developing muscle damage, injury, or atrophy. In some cases, the muscle damage, injury or atrophy is selected from the group consisting of acute muscle injury or trauma, soft, tissue hand injury, 15-PGDH elevation may occur in diseases of injured, damaged, aged, or diseased tissues and/or organs including skeletal muscle diseases (e.g., spinal muscular atrophy (SMA). Duchenne muscular dystrophy (DMD). Becker muscular dystrophy, limb girdle muscular dystrophy, amyotrophic lateral sclerosis (ALS), distal muscular dystrophy (DD), inherited myopathies, myotonic muscular dystrophy (MOD), mitochondrial myopathies, myotubular myopathy (MM), myasthenia gravis (MG), congestive heart failure, periodic paralysis, polymyositis, rhabdomyolysis, dermatomyositis, cancer cachexia. AIDS cachexia, cardiac cachexia, stress induced urinary incontinence, and sarcopenia), cardiovascular diseases (e.g., atrial fibrillation, stroke, ischemic heart diseases, cardiomyopathies, endocarditis, intracerebral hemorrhage), chronic respiratory diseases (e.g., chronic obstructive pulmonary disease, asbestosis, silicosis), nutritional diseases (trachoma, diarrheal diseases, encephalitis), kidney diseases (e.g., chronic kidney diseases), gastrointestinal and digestive diseases (e.g., NASH, pancreatitis, ulcer, intestinal obstruction), neurological disorders (e.g., Alzheimer's, dementia, Parkinson's), sensory disorders (e.g., hearing loss, macular degeneration, glaucoma), skin and subcutaneous diseases (e.g., cellulitis, ulcer, fungal skin diseases, pyoderma), osteoporosis, osteoarthritis, rheumatoid arthritis and the like. In addition, genetic disorders of these tissues that lead to premature aging syndromes, such as Bloom syndrome, Cockayne Syndrome, Hutchinson-Gilford Progeria Syndrome, Mandibuloacral Dysplasia, Progeria, Progeroid Syndrome, Rothmund-Thomson Syndrome, Seip Syndrome, Weiner Syndrome, Down Syndrome, Acrogeria, and Rothmund-Thomson syndrome, as well as immunodeficiencies of these tissues that lead to premature aging syndromes, such as Ataxia telangiectasia, and infectious diseases of these tissues that lead to premature aging syndromes, such as human immunodeficiency virus (HIV), can also benefit from 15-PGDH inhibition. 15-PGDH elevation in tissues and/or organs may also occur naturally during the aging process, and the methods and compositions provided herein contemplate treating a subject having injured, damaged, aged, or diseased tissues and/or organs with a 15-PGDH inhibitor and/or PGE2 activator, or by treating the injured, damaged, aged, or diseased tissue and/or organ itself with a 15-PGDH inhibitor and/or PGE2 activator, thereby regenerating and/or rejuvenating the injured, damaged, aged, or diseased tissue and/or organ.

Treating (e.g., skeletal muscle and/or non-skeletal muscle) tissues and/or organs with inhibitors of 15-PGDH may provide numerous advantages, such as that the treatment can be localized to specific cell types that express elevated levels of the enzyme (e.g., damaged, injured, diseased, or aged (e.g., skeletal muscle and/or non-skeletal muscle) tissues and/or organs), that it provides the ability to restore endogenous levels of PGE2 and/or PGD2 to achieve physiological “youthful” levels of PGE2 and/or PGD2, that it can target (e.g., skeletal muscle and/or non-skeletal muscle) tissues and/or organs with high senescent cell infiltration (e.g., colon, skin, spleen), which is thought to have detrimental effects in aging and aging-associated conditions, and that it provides the possibility of targeting 15-PGDH with molecules with relatively long half-lives or by using gene therapy, in order to provide sustained, systemic PGE2 and/or PGD2 benefits.

### 2. General

Practicing the methods disclosed herein utilizes routine techniques in the field of molecular biology. Basic texts disclosing the general methods of use described herein include Sambrook and Russell, Molecular Cloning, A Laboratory Manual (3rd ed. 2001): Kriegler, Gene Transfer and Expression: A Laboratory Manual (1990); and Current Protocols in Molecular Biology (Ausubel et al., eds., 1994)).

For nucleic acids, sizes are given in either kilobases (kb), base pairs (bp), or nucleotides (nt). Sizes of single-stranded DNA and/or RNA can be given in nucleotides. These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage and Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J. Chron. 255: 137-149 (1983).

### 3. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

“Age-related condition” or “age-related disease” refers to any disease, condition, or disorder that shows or potentially shows any signs or features associated with increasing age or passage of time in (e.g., skeletal muscle and/or non-skeletal muscle) tissues and/or organs, including, e.g., loss or decrease of tissue and/or organ function, loss or decrease of tissue and/or organ health, loss or decrease of one or more physiological activities of the tissue and/or organ, decreased protein synthesis in cells of the tissue and/or organ, increased protein degradation in cells of the tissue and/or organ, decreased survival or viability of the tissue and/or organ, decreased proliferation of cells within the tissue and/or organ, shortened telomeres in cells of the tissue and/or organ, mitochondrial dysfunction in cells of the tissue and/or organ, increased presence of senescent cells in the tissue and/or organ, increased levels of 15-PGDH levels and/or activity in the tissue and/or organ, decreased levels of PGE2 and/or PGD2 in the tissue and/or organ, etc. The condition or disease can be a result of natural aging processes due to the passage of time, of other factors such as lifestyle factors or disease, e.g., infectious disease, or of genetic conditions that cause premature aging.

A “skeletal muscle and/or non-skeletal muscle tissue” or “skeletal muscle and/or non-skeletal muscle organ” as used herein can refer to any tissue or organ in the body, including skeletal muscle (e.g., musculi pectoralis complex, latissimus dorsi, teres major and subscapularis, brachioradialis, biceps, brachialis, pronator quadratus, pronator teres, flexor carpi radialis, flexor carpi ulnaris, flexor digitorum superficialis, flexor digitorum profundus, flexor pollicis brevis, opponens pollicis, adductor pollicis, flexor pollicis brevis, iliopsoas, psoas, rectus abdominis, rectus femoris, gluteus maximus, gluteus medius, medial hamstrings, gastrocnemius, lateral hamstring, quadriceps mechanism, adductor longus, adductor brevis, adductor magnus, gastrocnemius medial, gastrocnemius lateral, soleus, tibialis posterior, tibialis anterior, flexor digitorum longus, flexor digitorum brevis, flexor hallucis longus, extensor hallucis longus, ocular muscles, pharyngeal muscles, sphincter muscles, hand muscles, arm muscles, foot muscles, leg muscles, chest muscles, stomach muscles, back muscles, buttock muscles, shoulder muscles, head and neck muscles), and can encompass tissues (e.g., groups of cells that have similar structure and function together as a unit) and organs (e.g., two or more tissues that function in a particular manner), as well as particular cell types within an organ or tissue. For example, a “skeletal muscle and/or non-skeletal muscle tissue” or “skeletal muscle and/or non-skeletal muscle organ” can include any of the following: skeletal muscle tissue, epithelial tissue, nerve tissue, connective tissue, smooth muscle, cardiac muscle, epidermal tissue, vascular tissue, heart, kidney, brain, bone, cartilage, brown fat, spleen, liver, colon, sensory organs, thyroid, lung, blood, small intestine, dental tissue, ovaries or other reproductive tissue or organs, hair, cochlea, oligodendrocytes, and combinations thereof.

The terms “damaged tissue”, “damaged organ”, “injured tissue”, “injured organ”, diseased tissue”, “diseased organ”, “aged tissue” and “aged organ” as used herein refer to any tissue or organ (e.g., skeletal muscle and/or non-skeletal muscle tissue or skeletal muscle and/or non-skeletal muscle organ) that exhibits one or more characteristics of a tissue or organ affected by damage, injury, disease, or an age-related condition, an age-related disease or disorder, and/or by the natural aging processes due to the passage of time. In some cases, the injured, damaged, aged, or diseased tissue or injured, damaged, aged, or diseased organ has increased levels and/or activity of 15-PGDH. In some cases, the injured, damaged, aged, or diseased tissue or injured, damaged, aged, or diseased organ has decreased levels of PGE2 and/or PGD2.

The terms “prostaglandin E2”, “PGE2”, and “dinoprostone” are used interchangeably herein and refer to prostaglandin that can be synthesized from arachidonic acid via cyclooxygenase (COX) enzymes and terminal prostaglandin E synthases (PGES). PGE2 plays a role in a number of biological functions including vasodilation, inflammation, and modulation of sleep/wake cycles. Structural and functional information about PGE2 can be found, e.g., in the entry for “Dinoprostone” of PubChem: pubchem.ncbi.nlm.nih.gov/compound/Dinoprostone, the contents of which are herein incorporated by reference in their entirety.

The terms “prostaglandin D2” or “PGD2” are used interchangeably herein and refer to prostaglandin that can be synthesized from arachidonic acid via cyclooxygenase (COX) enzymes and PGD2 synthases (PTDS). PGD2 is a structural isomer of PGE2, with the 9-keto and 11-hydroxy group on PGE2 reversed on PGD2. PGD2 plays a role in a number of biological functions including vasoconstriction, inflammation, the regulation of body temperature during sleep, chemotaxis, and male sexual development. Structural and functional information about PGD2 can be found, e.g., in the entry for “Prostaglandin D2” of PubChem: pubchem.ncbi.nlm.nih.gov/compound/448457, the contents of which are herein incorporated by reference in their entirety.

“15-PGDH” (15-hydroxyprostaglandin dehydrogenase) is an enzyme involved in the inactivation of a number of active prostaglandins, e.g., by catalyzing oxidation of PGE2 to 15-keto-prostaglandin E2 (15-keto-PGE2), or the oxidation of PGD2 to 15-keto-prostaglandin D2 (15-keto-PGD2). The human enzyme is encoded by the HPGD gene (Gene ID: 3248). The enzyme is a member of the short-chain nonmetalloenzyme alcohol dehydrogenase protein family. Multiple isoforms of the enzyme exist, e.g., in humans, any of which can be targeted using the present methods. For example, any of human isoforms 1-6 (e.g., GenBank Accession Nos. NP_000851.2, NP_001139288.1. NP_001243236.1, NP_001243234.1, NP_001243235.1, NP_001350503.1, NP_001243230.1) can be targeted, as can any isoform with 50%, 60%, 70%, 80%, 85%, 90%, 95%, 99%, or higher identity to the amino acid sequences of any of GenBank Accession Nos. NP_000851.2, NP_001139288.1, NP_001243236.1, NP_001243234.1, NP_001243235.1, NP_001350503.1, NP_001243230.1, or of any other 15-PGDH enzyme.

“Prostaglandin-E synthases” (PGES) are enzymes (isomerases) that catalyze the conversion of prostaglandin-H (PGH) into PGE2. Cytosolic PGES (cPGES) is a cytosolic protein that is constitutively expressed in a wide variety of cells and tissues and is associated with heat shock protein 90 (Hsp90). Membrane-associated PGES (mPGES), the expression of which is stimulus-inducible and is downregulated by anti-inflammatory glucocorticoids, is a perinuclear protein belonging to the microsomal glutathione S-transferase (GST) family. These two PGESs display distinct functional coupling with upstream COXs in cells: cPGES is predominantly coupled with the constitutive COX-1, whereas mPGES is preferentially linked with the inducible COX-2. Several cytosolic OSTs also have the capacity to convert PGF12 to PGE2 in vitro. The human enzyme is encoded by, for example, the PTGES (HGNC: 9599: NCBI Entrez Gene: 9536; Ensembl: ENSG00000148334; OMIM: 605172; UniProtKB/Swiss-Prot: 014684) and PMES2 genes (HGNC: 17822: NCBI Entrez Gene: 80142: Ensembl: ENSG00000148334: OMIM: 608152; UniProtKB/Swiss-Prot: Q9H7Z7). Any of the human PGES enzymes can be targeted, as can any isoform with 50%, 60%, 70%, 80%, 85%, 90%, 95%, 99%, or higher identity to the amino acid sequences of any of the prostaglandin-E synthase enzymes, including prostaglandin-E synthase 1 and prostaglandin-E synthase 2.

“Cyclooxeygenases” or “COX” enzymes are also involved in the synthesis of prostaglandins, including PGE2, by oxygenation of arachidonic acid, DGLA or EPA and terminal prostaglandin synthases. Also known as prostaglandin-endoperoxide synthase (PTGS), cyclooxygenase is an enzyme (family of isozymes, EC 1.14.99.1) that is responsible for formation of prostanoids, including thromboxane and prostaglandins, from arachidonic acid. A member of the animal-type heme peroxidase family, it is also known as prostaglandin G/H synthase. The specific reaction catalyzed is the conversion from arachidonic acid to prostaglandin H2, via a short-living prostaglandin G2 intermediate

A “15-PGDH inhibitor and/or PGE2 activator” refers to any agent that is capable of inhibiting, reducing, decreasing, attenuating, abolishing, eliminating, slowing, and/or counteracting in any way any aspect of the expression, stability, and/or activity of 15-PGDH. A 15-PGDH inhibitor and/or PGE2 activator can, for example, reduce or increase any aspect of the expression, e.g., transcription, RNA processing, RNA stability, and/or translation of a gene encoding 15-PGDH, e.g., the human HPGD gene, or PGE2, e.g., prostaglandin-E synthases, for example, PTGES1 (prostaglandin E synthase 1) or PTGES2 (prostaglandin E synthase 2) gene, or associated upstream enzymes, including COX1 and COX2 (cyclooxygenase 1 and 2, respectively) by, e.g., 5%, 10%, 15%, 20%, 25%, 30%, 35%, 40%, 45%, 50%, 55%, 60%, 65%, 70%, 75%, 80%, 85%, 90%, 95%, or more as compared to a control, e.g., in the absence of the inhibitor, in vitro or in vivo. A 15-PGDH inhibitor and/or PGE2 activator can, for example, inhibit or enhance transcription factors that promote or enhance expression of the gene encoding 15-PGDH, e.g., the human HPGD gene, or PGE2, e.g. prostaglandin synthase genes, for example, PTGES1 (prostaglandin E synthase 1), PTGES2 (prostaglandin E synthase 2) gene, or associated upstream enzymes, including COX1 and COX2 (cyclooxygenase 1 and 2, respectively) by, e.g., 5%, 10%, 15%, 20%, 25%, 30%, 35%, 40%, 45%, 50%, 55%, 60%, 65%, 70%, 75%, 80%, 85%, 90%, 95%, or more as compared to a control, e.g., in the absence of the inhibitor, in vitro or in vivo. Similarly, a 15-PGDH inhibitor and/or PGE2 activator can, for example, reduce or increase the activity, e.g., enzymatic activity, of a 15-PGDH enzyme or PGE2 synthase by, e.g., 5%, 10%, 15%, 20%, 25%, 30%, 35%, 40%, 45%, 50%, 55%, 60%, 65%, 70%, 75%, 80%, 85%, 90%, 95%, or more as compared to a control, e.g., in the absence of the inhibitor, in vitro or in vivo. Further, a 15-PGDH inhibitor and/or PGE2 activator can, for example, reduce or increase the stability of a 15-PGDH enzyme or PGE2 synthase by, e.g., 5%, 10%, 15%, 20%, 25%, 30%, 35%, 40%, 45%, 50%, 55%, 60%, 65%, 70%, 75%, 80%, 85%, 90%, 95%, or more as compared to a control, e.g., in the absence of the inhibitor, in vitro or in vivo. A “15-PGDH inhibitor and/or PGE2 activator”, also referred to herein as an “15-PGDH agent”, a “I5-PGDH compound,” a “PGE2 agent” or a “PGE2 compound” can be any molecule, either naturally occurring or synthetic, e.g., peptide, protein, oligopeptide (e.g., from about 5 to about 25 amino acids in length, e.g., about 5, about 10, about 15, about 20, or about 25 amino acids in length), small molecule (e.g., an organic molecule having a molecular weight of less than about 2500 Daltons, e.g., less than about 2000, less than about 1000, or less than about 500 daltons), antibody, nanobody, polysaccharide, lipid, fatty acid, inhibitory RNA (e.g., siRNA, shRNA, microRNA), modified RNA, polynucleotide, oligonucleotide, e.g., peptide nucleic acid (PNA), an aptamer, antisense oligonucleotide, morpholino oligomer, affimer, drug compound, or other compound. In some embodiments, the inhibitory RNA (e.g., siRNA, shRNA, microRNA), modified RNA, polynucleotide, morpholino oligomer, affimer, aptamer or oligonucleotide may be at least about 5 nucleotides, 10 nucleotides, 15 nucleotides, 20 nucleotides, 25 nucleotides, 30 nucleotides, 35 nucleotides, 40 nucleotides, 45 nucleotides, or 50 nucleotides in length.

A “senolytic agent” refers to any agent that is capable of inducing the death of senescent cells, e.g., inducing the death of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more of a population of senescent cells, in vitro or in vivo. A non-limiting list of senolytic agents that can be used in the present methods include Bcl2 inhibitors (e.g., navitoclax (ABT-263), ABT-737), pan-tyrosine kinase inhibitors (e.g., dasatinib), flavonoids (e.g., quercetin), peptides that interfere with the FOXO4-p53 interaction (e.g., FOXO4-DRI), a selective targeting system of senescent cells using galactooligosaccharide-coated nanoparticles, HSP90 inhibitors (e.g., 17-DMAG), and combinations thereof. In particular embodiments, a senolytic agent is capable of inducing the death of, e.g., 5%, 10%, 15%, 20%, 25%, 30%, 35%, 40%, 45%, 50%, 55%, 60%, 65%, 70%, 75%, 80%, 85%, 90%, 95% or more senescent cells, e.g., macrophages and/or fibroadipocytes within (e.g., skeletal muscle and/or non-skeletal muscle) tissue and/or organs.

The terms “expression” and “expressed” refer to the production of a transcriptional and/or translational product, e.g., of a nucleic acid sequence encoding a protein (e.g., 15-PGDH) or signaling molecule (e.g. PGE2). In some embodiments, the term refers to the production of a transcriptional and/or translational product encoded by a gene (e.g., the human HPGD gene) or a portion thereof. The level of expression of a DNA molecule in a cell may be assessed on the basis of either the amount of corresponding mRNA that is present within the cell or the amount of protein encoded by that DNA produced by the cell. In some embodiments, the term refers to the synthesis of a signaling product, e.g., PGE2 synthesis from arachidonic acid through expression of prostaglandin synthases (e.g., prostaglandin-E synthases, including prostaglandin-E synthase-1 and prostaglandin-E synthase 2, and COX1 and COX2 enzymes).

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. The term includes antibody fragments having the same antigen specificity, and fusion products thereof.

An exemplary immunoglobulin (antibody) structural unit comprises a tetramer. Each tetramer is composed of two identical pairs of polypeptide chains, each pair having one “light” chain (about 25 kDa) and one “heavy” chain (about 50-70 kDa). The N-terminus of each chain defines a variable region of about 100 to 110 or more amino acids primarily responsible for antigen recognition. Thus, the terms “variable heavy chain,” “VH”, or “VH” refer to the variable region of an immunoglobulin heavy chain, including an Fv, scFv, dsFv or Fab; while the terms “variable light chain,” “V_{H}”, or “VH” refer to the variable region of an immunoglobulin light chain, including of an Fv, scFv, dsFv or Fab. Equivalent molecules include antigen binding proteins having the desired antigen specificity, derived, for example, by modifying an antibody fragment or by selection from a phage display library.

The terms “antigen-binding portion” and “antigen-binding fragment” are used interchangeably herein and refer to one or more fragments of an antibody that retains the ability to specifically bind to an antigen (e.g., a 15-PGDH protein or prostaglandin-E synthases).

Examples of antibody-binding fragments include, but are not limited to, a Fab fragment (a monovalent fragment consisting of the VL, VH, CL, and CHI domains), F(ab′)₂ fragment (a bivalent fragment comprising two Fab fragments linked by a disulfide bridge at the hinge region), a single chain Fv (scFv), a disulfide-linked Fv (dsFv), complementarity determining regions (CDRs), VL (light chain variable region), VH (heavy chain variable region), nanobodies, and any combination of those or any other functional portion of an immunoglobulin peptide capable of binding to target antigen (see, e.g., Fundamental Immunology (Paul ed., 4th ed. 2001).

The phrase “specifically binds” refers to a molecule (e.g., a 15-PGDH inhibitor and/or PGE2 activator) that binds to a target with greater affinity, avidity, more readily, and/or with greater duration to that target in a sample than it binds to a non-target compound. In some embodiments, a molecule that specifically binds a target (e.g., 15-PGDH, PGE2 or prostaglandin-E synthases) binds to the target with at least 2-fold greater affinity than non-target compounds, e.g., at least 3-fold, at least 4-fold, at least 5-fold, at least 6-fold, at least 7-fold, at least 8-fold, at least 9-fold, at least 10-fold, at least 20-fold, at least 25-fold, at least 50-fold or greater affinity. For example, in some embodiments, a molecule that specifically binds to 15-PGDH or PGE2 typically binds to 15-PGDH or PGE2 with at least a 2-fold greater affinity than to a non-15-PGDH or PGE2 target.

The term “derivative,” in the context of a compound, includes but is not limited to, amide, ether, ester, amino, carboxyl, acetyl, and/or alcohol derivatives of a given compound.

The term “treating” or “treatment” refers to any one of the following: ameliorating one or more symptoms of a disease or condition: preventing the manifestation of such symptoms before they occur; slowing down or completely preventing the progression of the disease or condition (as may be evident by longer periods between reoccurrence episodes, slowing down or prevention of the deterioration of symptoms, etc.); enhancing the onset of a remission period: slowing down the irreversible damage caused in the progressive-chronic stage of the disease or condition (both in the primary and secondary stages): delaying the onset of said progressive stage, or any combination thereof.

The term “administer”, “administering”, or “administration” refers to the methods that may be used to enable delivery of agents or compositions such as the compounds described herein to a desired site of biological action. These methods include, but are not limited to, parenteral administration (e.g., intravenous, subcutaneous, intraperitoneal, intramuscular, intra-arterial, intravascular, intracardiac, intrathecal, intranasal, intradermal, intravitreal, and the like), transmucosal injection, oral administration, administration as a suppository, and topical administration. In some cases, the administration is systemic administration (e.g., administration into the circulatory system such that multiple tissues and/or organs are treated or affected). In some cases, the administration is local administration (e.g., directly to the tissue or organ such that the tissue and/or organ is treated or affected). One skilled in the art will know of additional methods for administering a therapeutically effective amount of the compounds described herein.

The term “therapeutically effective amount” or “therapeutically effective dose” or “effective amount” refers to an amount of a compound (e.g., 15-PGDH inhibitor and/or PGE2 activator) that is sufficient to bring about a beneficial or desired clinical effect. A therapeutically effective amount or dose may be based on factors individual to each patient, including, but not limited to, the patient's age, size, type or extent of disease or condition, stage of the disease or condition, route of administration, the type or extent of supplemental therapy used, and/or ongoing disease process and type of treatment desired (e.g., aggressive vs. conventional treatment). Therapeutically effective amounts of a compound (e.g., 15-PGDH inhibitor and/or PGE2 activator), as described herein, can be estimated initially from cell culture and animal models. For example, IC₅₀ values determined in cell culture methods can serve as a starting point in animal models, while IC₅₀ values determined in animal models can be used to find a therapeutically effective dose in humans.

The term “pharmaceutical composition” as used herein refers to a composition comprising a compound (e.g., 15-PGDH inhibitor and/or PGE2 activator) as described herein and one or more pharmaceutically acceptable carriers and/or pharmaceutically acceptable excipients.

The term “pharmaceutically acceptable carrier” as used herein refers to a carrier or a diluent that does not cause significant irritation to an organism and does not abrogate the biological activity and properties of the administered compound.

The terms “subject”, “individual”, and “patient” are used interchangeably herein to refer to a vertebrate, preferably a mammal, more preferably a human. Mammals include, but are not limited to, murines, rats, simians, humans, farm animals or livestock for human consumption such as pigs, cattle, and ovines, as well as sport animals and pets. Subjects also include vertebrates such as fish and poultry.

The term “acute regimen”, in the context of administration of a compound, refers to a temporary or brief application of a compound to a subject, e.g., human subject, or to a repeated application of a compound to a subject, e.g., human subject, wherein a desired period of time (e.g., 1 day) lapses between applications. In some embodiments, an acute regimen includes an acute exposure (e.g., a single dose) of a compound to a subject over the course of treatment or over an extended period of time. In other embodiments, an acute regimen includes intermittent exposure (e.g., repeated doses) of a compound to a subject in which a desired period of time lapses between each exposure.

The term “chronic regimen,” in the context of administration of a compound, refers to a repeated, chronic application of a compound to a subject, e.g., human subject, over an extended period of time such that the amount or level of the compound is substantially constant over a selected time period. In some embodiments, a chronic regimen includes a continuous exposure of a compound to a subject over an extended period of time.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. The promoter can be a heterologous promoter. In the context of promoters operably linked to a polynucleotide, a “heterologous promoter” refers to a promoter that would not be so operably linked to the same polynucleotide as found in a product of nature (e.g., in a wild-type organism).

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. In particular embodiments, modified RNA molecules are used, e.g., mRNA with certain chemical modifications to allow increased stability and/or translation when introduced into cells, as described in more detail below. It will be appreciated that any of the RNAs used in the present methods, including nucleic acid inhibitors such as siRNA or shRNA, can be used with chemical modifications to enhance, e.g., stability and/or potency, e.g., as described in Dar et al. (2016) Scientific Reports 6: article no. 20031 (2016), and as presented in the database accessible at crdd.osdd.net/servers/sirnamod/.

“Polypeptide”, “peptide”, and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

As used herein, the terms “identical” or percent “identity”, in the context of describing two or more polynucleotide or amino acid sequences, refer to two or more sequences or specified subsequences that are the same. Two sequences that are “substantially identical” have at least about 60% identity, preferably at least about 65%, at least about 70%, at least about 75%, at least about 80%, at least about 85%, at least about 90%, at least about 91%, at least about 92%, at least about 93, at least about 94%, at least about 95%, at least about 96%, at least about 97%, at least about 98%, at least about 99%, or about 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. With regard to amino acid sequences, in some cases, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm and the default parameters are used.

The terms “regenerating and/or rejuvenating” or “regeneration or rejuvenation” as used herein refers to the restoration or revitalization of an individual, a cell, a tissue, and/or an organ (e.g., to a more “youthful” state, e.g., to more closely resemble a young individual, young cell, young tissue, and/or young organ). In some cases, the terms “regenerating and/or rejuvenating” or “regeneration or rejuvenation” may refer to increasing, enhancing, restoring, regenerating, and/or improving a function of a (e.g., injured, damaged, aged, or diseased) tissue and/or (e.g., injured, damaged, aged, or diseased) organ (e.g., to more closely resemble a function of a young tissue and/or young organ). In some cases, the terms “regenerating and/or rejuvenating” or “regeneration or rejuvenation” may refer to increasing, enhancing, restoring, regenerating, and/or improving a structure of a (e.g., injured, damaged, aged, or diseased) tissue and/or (e.g., injured, damaged, aged, or diseased) organ (e.g., to more closely resemble a structure of a young tissue and/or young organ). In some cases, the terms “regenerating and/or rejuvenating” or “regeneration or rejuvenation” may refer to decreasing levels of 15-PGDH and/or activity of 15-PGDH, and/or increasing levels of PGE2 and/or PGD2 in a (e.g., injured, damaged, aged, or diseased) tissue or (e.g., injured, damaged, aged, or diseased) organ (such that the levels of 15-PGDH and/or activity of 15-PGDH and/or levels of PGE2 and/or PGD2 more closely resemble those of young tissue or young organs).

### 4. Methods of Regenerating and/or Rejuvenating Tissue and/or Organ Function in Subjects with Age-Related Conditions

In another embodiment, a method is provided for regenerating and/or rejuvenating an injured, damaged, aged, or diseased tissue and/or organ in a subject, the method comprising: administering to the subject an amount of a 15-PGDH inhibitor and/or PGE2 activator effective to inhibit 15-PGDH and/or increase PGE2 synthesis, thereby regenerating and/or rejuvenating the injured, damaged, aged, or diseased tissue and/or organ. In one embodiment, a method is provided for regenerating and/or rejuvenating a function of an injured, damaged, aged, or diseased (e.g., skeletal muscle and/or non-skeletal muscle) tissue or injured, damaged, aged, or diseased (e.g., skeletal muscle and/or non-skeletal muscle) organ in an individual, the method comprising: administering to the subject a 15-PGDH inhibitor and/or PGE2 activator in an amount effective to inhibit 15-PGDH expression and/or activate or increase prostaglandin synthase expression and/or increase PGE2 levels and/or reduce 15-PGDH levels in the individual, thereby regenerating and/or rejuvenating the function of the injured, damaged, aged, or diseased (e.g., skeletal muscle and/or non-skeletal muscle) tissue or the injured, damaged, aged, or diseased (e.g., skeletal muscle and/or non-skeletal muscle) organ in the individual.

Further provided herein is a method of enhancing a function of a (e.g., skeletal muscle and/or non-skeletal muscle) tissue or (e.g., skeletal muscle and/or non-skeletal muscle) organ in a subject, the method comprising: administering to the subject a 15-PGDH inhibitor and/or PGE2 activator in an amount effective to inhibit 15-PGDH expression and/or activate or increase prostaglandin synthase expression and/or increase PGE2 levels and/or reduce 15-PGDH levels in the (e.g., skeletal muscle and/or non-skeletal muscle) tissue or (e.g., skeletal muscle and/or non-skeletal muscle) organ, thereby enhancing a function of the (e.g., skeletal muscle and/or non-skeletal muscle) tissue or (e.g., skeletal muscle and/or non-skeletal muscle) organ in the subject. In some cases, the subject is less than 30 years of age (e.g., 29, 28, 27, 26, 25, 24, 23, 22, 21, 20, 19, 18, 17, 16, 15, 14, 13, 12, 11, 10, 9, 8, 7, 6, 5, 4, 3, 2, or 1 years of age). In other cases, the subject is greater than 30 years of age.

In various aspects, the injured, damaged, aged, or diseased (e.g., skeletal muscle and/or non-skeletal muscle) tissue and/or organ may have one or more senescent cells (e.g., present within or near the injured, damaged, aged, or diseased tissue and/or organ). In some cases, the injured, damaged, aged, or diseased (e.g., skeletal muscle and/or non-skeletal muscle) tissue and/or organ may have a plurality of senescent cells (e.g., present within or near the injured, damaged, aged, or diseased tissue and/or organ). In some cases, the injured, damaged, aged, or diseased (e.g., skeletal muscle and/or non-skeletal muscle) tissue and/or organ may have an increased accumulation of senescent cells (e.g., within or near the injured, damaged, aged, or diseased (e.g., skeletal muscle and/or non-skeletal muscle) tissue and/or organ) (e.g., relative to young (e.g., skeletal muscle and/or non-skeletal muscle) tissue and/or organ). In some cases, the injured, damaged, aged, or diseased (e.g., skeletal muscle and/or non-skeletal muscle) tissue and/or organ may have a number of senescent cells that is higher (e.g., substantially higher) than a number typically found in young (e.g., skeletal muscle and/or non-skeletal muscle) tissue and/or organs. The senescent cells may express one or more senescent markers. The senescent cells may have an increased level of one or more senescent markers relative to a non-senescent cell. The one or more senescent markers may be, without limitation, p15Ink4b, p16Ink4a, p19Arf, p21, Mmp13, II1a, II1b, and I16. In various aspects, the subject may be selected for treatment (e.g., by any method disclosed herein) based on a level of senescent cells present within the injured, damaged, aged, or diseased (e.g., skeletal muscle and/or non-skeletal muscle) tissue and/or organ and/or based on the presence or levels of one or more senescent markers. In some cases, the presence of senescent cells within the injured, damaged, aged, or diseased (e.g., skeletal muscle and/or non-skeletal muscle) tissue and/or organ (e.g., at a number higher than a number typically found in young (e.g., skeletal muscle and/or non-skeletal muscle) tissue and/or organs) and/or the presence and/or levels of one or more senescent markers may indicate that a treatment (e.g., any disclosed herein) is likely to provide a therapeutic benefit. In some cases, the senescent cells may express 15-PGDH (e.g., at levels effective to decrease a level of PGE2 within the injured, damaged, aged, or diseased (e.g., skeletal muscle and/or non-skeletal muscle) tissue and/or organ). In some cases, the senescent cells may be macrophages.

In various aspects, the subject may express one or more biomarkers of aging. A biomarker of aging may include, without limitation, an increase in 15-PGDH levels (e.g., relative to a level present in young (e.g., skeletal muscle and/or non-skeletal muscle) tissue and/or organs), a decrease in PGE2 levels (e.g., relative to a level present in young (e.g., skeletal muscle and/or non-skeletal muscle) tissue and/or organs), an increase in a PGE2 metabolite (e.g., relative to a level present in young (e.g., skeletal muscle and/or non-skeletal muscle) tissue and/or organs), an increase or a greater accumulation of senescent cells (e.g., relative to a level present in young (e.g., skeletal muscle and/or non-skeletal muscle) tissue and/or organs), an increase in expression of one or more atrogenes (e.g., Atrogin1 (MAFbx1). Fbxo30 (MuSA), and Trim63 (MuRF1)) (e.g., relative to a level present in young (e.g., skeletal muscle and/or non-skeletal muscle) tissue and/or organs), a decrease in mitochondria biogenesis and/or function (e.g., relative to a level present in young (e.g., skeletal muscle and/or non-skeletal muscle) tissue and/or organs), and an increase in transforming growth factor pathway signaling (e.g., an increase in expression of one or more genes involved in a transforming growth factor signaling pathway, e.g., one or more of Activin receptor, Myostatin, a SMAD protein, and a bone morphogenetic protein) (e.g., relative to a level present in young (e.g., skeletal muscle and/or non-skeletal muscle) tissue and/or organs). In some cases, a biomarker of aging may include increased levels or activity of 15-PGDH (e.g., within the injured, damaged, aged, or diseased (e.g., skeletal muscle and/or non-skeletal muscle) tissue and/or organ) (e.g., relative to a level present in young (e.g., skeletal muscle and/or non-skeletal muscle) tissue and/or organs). In some cases, a biomarker of aging may include decreased levels of PGE2 (e.g., within the injured, damaged, aged, or diseased (e.g., skeletal muscle and/or non-skeletal muscle) tissue and/or organ) (e.g., relative to a level present in young (e.g., skeletal muscle and/or non-skeletal muscle) tissue and/or organs). In some cases, a biomarker of aging may include increased levels of a PGE2 metabolite (e.g., 15-keto PGE2 and 13,14-dihydro-15-keto PGE2, e.g., within the injured, damaged, aged, or diseased (e.g., skeletal muscle and/or non-skeletal muscle) tissue and/or organ) (e.g., relative to a level present in young (e.g., skeletal muscle and/or non-skeletal muscle) tissue and/or organs). In some cases, the presence of a biomarker of aging may indicate that the subject is likely to benefit from treatment according to any method disclosed herein. Young (e.g., skeletal muscle and/or non-skeletal muscle) tissue and/or organs may include (e.g., skeletal muscle and/or non-skeletal muscle) tissue and/or organs from a subject under the age of 30 (e.g., 29, 28, 27, 26, 25, 24, 23, 22, 21, 20, 19, 18, 17, 16, 15, 14, 13, 12, 11, 10, 9, 8, 7, 6, 5, 4, 3, 2, or 1 years of age).

In various aspects, levels of PGE2 present within the injured, damaged, aged, or diseased (e.g., skeletal muscle and/or non-skeletal muscle) tissue and/or organ may be increased (e.g., after treatment with a 15-PGDH inhibitor and/or PGE2 activator, e.g., according to methods provided herein) relative to levels present in the injured, damaged, aged, or diseased (e.g., skeletal muscle and/or non-skeletal muscle) tissue and/or organ prior to the treatment (e.g., with the 15-PGDH inhibitor and/or PGE2 activator). PGE2 levels in the injured, damaged, aged, or diseased (e.g., skeletal muscle and/or non-skeletal muscle) tissue and/or organ may be increased (e.g., by any method disclosed herein) by at least about 5%, by at least about 10% (e.g., at least about 15%, at least about 20%, at least about 25%, at least about 30%, at least about 40%, at least about 50%, at least about 60%, at least about 70%, or greater) relative to levels present in the injured, damaged, aged, or diseased (e.g., skeletal muscle and/or non-skeletal muscle) tissue and/or organ prior to the treatment (e.g., with the 15-PGDH inhibitor and/or PGE2 activator). In various aspects, levels of PGE2 present within the injured, damaged, aged, or diseased (e.g., skeletal muscle and/or non-skeletal muscle) tissue and/or organ may be increased (e.g., after treatment with a 15-PGDH inhibitor and/or PGE2 activator, e.g., according to methods provided herein) to a level substantially similar to a level present in young (e.g., skeletal muscle and/or non-skeletal muscle) tissue and/or organs. PGE2 levels in the injured, damaged, aged, or diseased (e.g., skeletal muscle and/or non-skeletal muscle) tissue and/or organ may be increased (e.g., by any method disclosed herein) to a level within about 25% or within about 50% or less of a level present in young (e.g., skeletal muscle and/or non-skeletal muscle) tissue and/or organs (e.g., within about 40%, within about 35%, within about 30%, within about 25%, within about 20%, within about 15%, within about 10%, within about 5%, or within about 1%).

In various aspects, levels of PGE2 metabolites present within the injured, damaged, aged, or diseased (e.g., skeletal muscle and/or non-skeletal muscle) tissue and/or organ may be decreased (e.g., after treatment with a 15-PGDH inhibitor, e.g., according to methods provided herein) relative to levels present in the injured, damaged, aged, or diseased (e.g., skeletal muscle and/or non-skeletal muscle) tissue and/or organ prior to the treatment (e.g., with the 15-PGDH inhibitor). PGE2 metabolite levels in the injured, damaged, aged, or diseased (e.g., skeletal muscle and/or non-skeletal muscle) tissue and/or organ may be decreased (e.g., by any method disclosed herein) by at least about 5%, by at least about 10% (e.g., at least about 15%, at least about 20%, at least about 25%, at least about 30%, at least about 40%, at least about 50%, at least about 60%, at least about 70%, or greater) relative to levels present in the injured, damaged, aged, or diseased (e.g., skeletal muscle and/or non-skeletal muscle) tissue and/or organ prior to the treatment (e.g., with the 15-PGDH inhibitor). In various aspects, levels of PGE2 metabolites present within the injured, damaged, aged, or diseased (e.g., skeletal muscle and/or non-skeletal muscle) tissue and/or organ may be decreased (e.g., after treatment with a 15-PGDH inhibitor, e.g., according to methods provided herein) to a level substantially similar to a level present in young (e.g., skeletal muscle and/or non-skeletal muscle) tissue and/or organs. PGE2 metabolite levels in the injured, damaged, aged, or diseased (e.g., skeletal muscle and/or non-skeletal muscle) tissue and/or organ may be decreased (e.g., by any method disclosed herein) to a level within about 25% or within about 50% or less of a level present in young (e.g., skeletal muscle and/or non-skeletal muscle) tissue and/or organs (e.g., within about 40%, within about 35%, within about 30%, within about 25%, within about 20%, within about 15%, within about 10%, within about 5%, or within about 1%). The PGE2 metabolite may be 15-keto PGE2, 13,14-dihydro-15-keto PGE2, or both. The PGE2 metabolite may be 15-keto PGE2, 13,14-dihydro-15-keto PGE2, or both.

In various aspects, a function of the injured, damaged, aged, or diseased (e.g., skeletal muscle and/or non-skeletal muscle) tissue and/or organ may be enhanced (e.g., after treatment with a 15-PGDH inhibitor and/or PGE2 activator, e.g., according to methods provided herein) relative to the injured, damaged, aged, or diseased (e.g., skeletal muscle and/or non-skeletal muscle) tissue and/or organ prior to the treatment (e.g., with the 15-PGDH inhibitor and/or PGE2 activator). A function of the injured, damaged, aged, or diseased (e.g., skeletal muscle and/or non-skeletal muscle) tissue and/or organ may be enhanced (e.g., by any method disclosed herein) by at least about 5%, by at least about 10% (e.g., at least about 15%, at least about 20%, at least about 25%, at least about 30%, at least about 40%, at least about 50%, at least about 60%, at least about 70%, or greater) relative to levels present in the injured, damaged, aged, or diseased (e.g., skeletal muscle and/or non-skeletal muscle) tissue and/or organ prior to the treatment (e.g., with the 15-PGDH inhibitor and/or PGE2 activator). In various aspects, a function of the injured, damaged, aged, or diseased (e.g., skeletal muscle and/or non-skeletal muscle) tissue and/or organ may be enhanced (e.g., after treatment with a 15-PGDH inhibitor and/or PGE2 activator, e.g., according to methods provided herein) to a level substantially similar to a level present in young (e.g., skeletal muscle and/or non-skeletal muscle) tissue and/or organs. A function of the injured, damaged, aged, or diseased (e.g., skeletal muscle and/or non-skeletal muscle) tissue and/or organ may be enhanced (e.g., by any method disclosed herein) to a level within about 25% or within about 50% or less of a level present in young (e.g., skeletal muscle and/or non-skeletal muscle) tissue and/or organs (e.g., within about 40%, within about 35%, within about 30%, within about 25%, within about 20%, within about 15%, within about 10%, within about 5%, or within about 1%). A function may include increased protein synthesis, increased cell proliferation, increased cell survival, decreased protein degradation, or any combination thereof. A function may include any function disclosed herein.

In some instances, treatment (e.g., with a 15-PGDH inhibitor and/or PGE2 activator, e.g., according to methods provided herein) may result in regeneration or rejuvenation of the injured, damaged, aged, or diseased (e.g., skeletal muscle and/or non-skeletal muscle) tissue and/or organ (e.g., an increase in one or more functions of the injured, damaged, aged, or diseased (e.g., skeletal muscle and/or non-skeletal) muscle tissue and/or organ) such that the injured, damaged, aged, or diseased (e.g., skeletal muscle and/or non-skeletal muscle) tissue and/or organ more closely resembles young (e.g., skeletal muscle and/or non-skeletal muscle) tissue and/or organs. In some embodiments, the function of the skeletal muscle may comprise one or more of muscle size, muscle fiber length, contraction rate, relaxation rate, maximum contraction velocity, isotonic strength, isometric force, muscle power, muscle fatigue, muscle quality based on maximal muscle force and power production relative to muscle size, and electrical signal transmission.

The present disclosure provides methods of increasing the function, health, and other properties of (e.g., skeletal muscle and/or non-skeletal muscle) tissues and/or organs in subjects, e.g., human subjects, with an age-related condition or disease, or due to the natural aging process, comprising administering a 15-PGDH inhibitor and/or PGE2 activator to the subject. The administration of the 15-PGDH inhibitor and/or PGE2 activator can be systemic or local, and can enhance any of a number of aspects of the tissue and/or organ including enhancing function, enhancing physiological activity, enhancing endurance, and/or enhancing performance on any assay for assessing tissue or organ function, or any other measure of tissue or organ function or health in the subject. In some embodiments, the administration of the 15-PGDH inhibitor and/or PGE2 activator results in protection against cell death in the (e.g., skeletal muscle and/or non-skeletal muscle) tissue and/or organ in the subject. In some embodiments, the administration of the 15-PGDH inhibitor and/or PGE2 activator results in reduced protein degradation in the (e.g., skeletal muscle and/or non-skeletal muscle) tissue and/or organ in the subject. In some embodiments, the administration of the 15-PGDH inhibitor and/or PGE2 activator results in increased protein synthesis in the (e.g., skeletal muscle and/or non-skeletal muscle) tissue and/or organ in the subject. In some embodiments, administration of the 15-PGDH inhibitor and/or PGE2 activator may result in increased endurance (e.g., during exercise, e.g., as measured on a treadmill). In some cases, the increased endurance of the subject (e.g., during exercise) may be due to an increased function and/or regeneration or rejuvenation of the injured, damaged, aged, or diseased (e.g., skeletal muscle and/or non-skeletal muscle) tissue and/or organ (e.g., heart, lungs, bones, etc.).

The present disclosure also provides methods of measuring 15-PGDH and PGE2 levels in (e.g., skeletal muscle and/or non-skeletal muscle) tissues and/or organs of a subject with an age-related condition or an aged subject. Such methods are useful, e.g., for the use of 15-PGDH and PGE2 as a biomarker of aging or aging (e.g., skeletal muscle and/or non-skeletal muscle) tissues and/or organs and/or for a loss or decrease of function of (e.g., skeletal muscle and/or non-skeletal muscle) tissues and/or organs, e.g., wherein an elevated level of 15-PDGH levels or activity or a depressed level of PGE2 levels or activity, e.g., an increase or decrease of about 5%, an increase or decrease of about 10%, about 20%, about 30%, about 40%, about 50%, about 60%, about 70%, about 80%, about 90%, about 100% or more relative to a control level in a subject without an age-related condition is indicative of aging or a loss or decrease of function in the tissue and/or organ. In such methods, 15-PGDH or PGE2 can be assessed in any of a number of ways, e.g., by detecting levels of a transcript encoding a 15-PGDH protein or prostaglandin synthases (e.g., prostaglandin-E synthases, including prostaglandin-E synthase 1 and prostaglandin-E synthase 2 and COX1 and COX2 enzymes), by detecting levels of a 15-PGDH polypeptide or PGE2, or by detecting 15-PGDH or prostaglandin-E synthases enzymatic activity.

In particular embodiments, the inhibition of 15-PGDH in the subject leads to an increase in PGE2 and/or PGD2, e.g., an elevation, increase, or restoration of PGE2 and/or PGD2 levels, in the (e.g., skeletal muscle and/or non-skeletal muscle) tissue and/or organ of the subject, and a decrease in PGE2 and/or PGD2 metabolites such as 15-keto-PGE2, 13,14-dihydro-15-keto-PGE2 (PGEM), 15-keto-PGD2, and 13,14-Dihydro-15-keto-PGD2. In some embodiments, the inhibition also leads to increased signaling through PGE2 receptors, e.g., EP1, EP2, EP3, and/or EP4 (also known as Ptger1, Ptger2, Ptger3, Ptger4) in the (e.g., skeletal muscle and/or non-skeletal muscle) tissue and/or organ. In some embodiments, the inhibition also leads to increased signaling through PGD2 receptors, e.g., DP1 and/or DP2 (also known as PTGDR1, PTGDR2/CRTH2).

In particular embodiments, the herein-described benefits of 15-PGDH inhibitor and/or PGE2 activator administration in the (e.g., skeletal muscle and/or non-skeletal muscle) tissue and/or organ, e.g., enhanced tissue health, function, physiological activity, etc., occur independently of any regeneration of the tissue and/or organ in the subject. In other words, while there may be regeneration of the tissue and/or organ in the subject, e.g., if the tissue and/or organ has been injured or damaged, the herein-described effects do not require the regeneration and would occur even without the regeneration. In particular embodiments, the (e.g., skeletal muscle and/or non-skeletal muscle) tissue and/or organ is not injured or damaged and has not or does not undergo regeneration.

In some embodiments, the administration of the 15-PGDH inhibitor and/or PGE2 activator inhibits 15-PGDH activity or reduced 15-PGDH levels in senescent cells, e.g., macrophages, fibroadipocytes, other mononuclear interstitial tissue resident cells including other immune cells, fibroblasts, endothelial cells, preadipocytes, and/or adipocytes, within the (e.g., skeletal muscle and/or non-skeletal muscle) tissue and/or organ of the subject. Consequently, the administration of the 15-PGDH inhibitor and/or PGE2 activator may result in decreased fibrosis or may inhibit fibrosis in various injured, damaged, aged, or diseased tissues and/or organs (e.g., heart, lung, etc.). In some embodiments, a fibrotic disease includes primary biliary cholangitis (PBC), non-alchoholic steatohepatitis (NASH), scleroderma and pulmonary fibrosis, including idiopathic pulmonary fibrosis (IPF). In some embodiments, the administration of the 15-PGDH inhibitor and/or PGE2 activator reduces collagen synthesis, fibrillogenesis, and/or crosslinking. In some embodiments, the methods further comprise the administration of a senolytic agent to the subject. Examples of senolytic agents that can be used include, inter alia, Bc12 inhibitors such as navitoclax (also known as ABT-263) and ABT-737, pan-tyrosine kinase inhibitors such as dasatinib together with a flavonoid such as quercetin, a peptide which interferes with the FOXO4-p53 interaction such as FOXO4-DRI, a selective targeting system of senescent cells using galactooligosaccharides-coated nanoparticles, a combination drug therapy comprising dasatinib and quercetin, and HSP90 inhibitors such as 17-DMAG. It will be appreciated that the senolytic agent can be administered together with the 15-PGDH inhibitor and/or PGE2 activator, e.g., within a single pharmaceutical formulation, or separately.

Regenerating and/or Rejuvenating Injured, Damaged, Aged, or Diseased Skeletal Muscle Tissue

The methods provided herein can be used to prevent or treat a condition or disease associated with skeletal muscle damage, injury, or atrophy in a subject in need thereof. The method can provide prophylactic treatment to a subject who is likely to experience muscle damage, injury or atrophy. In some embodiments, the subject can have a condition or disease with possible secondary symptoms that affect skeletal muscle. In other embodiments, the subject has undergone a surgical or therapeutic intervention to treat the skeletal muscle condition or disease, and the method disclosed here is used to prevent or inhibit recurrence or relapse. In some embodiments, the subject has any one of the conditions or diseases described herein that affects skeletal muscle.

In various aspects, after administration of the 15-PGDH inhibitor and/or PGE2 activator, a function of the injured, damaged, aged, or diseased skeletal muscle is regenerated and/or rejuvenated relative to a function of the injured, damaged, aged, or diseased skeletal prior to treatment with the 15-PGDH inhibitor and/or PGE2 activator. In various aspects, after administration of the 15-PGDH inhibitor and/or PGE2 activator, a function of the injured, damaged, aged, or diseased skeletal muscle is regenerated and/or rejuvenated to a level substantially similar to a level found in young skeletal muscle tissue.

In some embodiments, after administration of the 15-PGDH inhibitor and/or PGE2 activator, the injured, damaged, aged, or diseased skeletal muscle exhibits increased or enhanced force or strength, as compared to the injured, damaged, aged, or diseased skeletal muscle prior to treatment with the 15-PGDH inhibitor and/or PGE2 activator. In some embodiments, after administration of the 15-PGDH inhibitor and/or PGE2 activator, the injured, damaged, aged, or diseased skeletal muscle exhibits increased force or strength that are substantially similar to levels found in young skeletal muscle. In some embodiments, after administration of the 15-PGDH inhibitor and/or PGE2 activator, the injured, damaged, aged, or diseased skeletal muscle exhibits increased endurance, as compared to the injured, damaged, aged, or diseased skeletal muscle prior to treatment with the 15-PGDH inhibitor and/or PGE2 activator. In some embodiments, after administration of the 15-PGDH inhibitor and/or PGE2 activator, the injured, damaged, aged, or diseased skeletal muscle tissue exhibits muscle endurance that is substantially similar to muscle mass levels found in young skeletal muscle tissue. In some embodiments, after administration of the 15-PGDH inhibitor and/or PGE2 activator, the injured, damaged, aged, or diseased skeletal muscle exhibits increased muscle mass, as compared to the injured, damaged, aged, or diseased skeletal muscle prior to treatment with the 15-PGDH inhibitor and/or PGE2 activator. In some embodiments, after administration of the 15-PGDH inhibitor and/or PGE2 activator, the injured, damaged, aged, or diseased skeletal muscle tissue exhibits muscle mass that is substantially similar to muscle mass levels found in young skeletal muscle tissue.

Regenerating and/or Rejuvenating Injured, Damaged, Aged or Diseased Spleen

The spleen is a secondary lymphoid organ consisting of two compartments—the blood-containing red pulp region where pathogens and aged erythrocytes are removed by macrophages and the white pulp region comprising of B and T cells responsible for the adaptive immune response. The white pulp region is surrounded by a marginal zone which is involved in innate and adaptive immunity. It is comprised of stromal cells associated with a subset of macrophages and B cells that enable the capture of blood borne antigens.

The spleen's main function is to filter blood. For example, the spleen filters old, damaged, or abnormal red blood cells (erythrocytes) from the blood. The spleen also filters microorganisms and pathogens from the blood, as well as cellular debris. The spleen also can store leftover used products including iron, which is stored in the form of ferritin or bilirubin and returned to the bone marrow for hemoglobin production. The spleen also can store blood and can release the blood in the case of excessive blood loss. The spleen is also responsible for maturation of lymphoid cell types involved in the adaptive immune response. The spleen is also involved in initiating immune reactions to blood-borne antigens.

During the aging process, one or more of these functions may be abrogated or reduced. In elderly people, splenic function has been shown to be decreased or impaired. In addition, injured, damaged, aged, or diseased spleens have been shown to exhibit abnormal morphology with aberrant lymphoid follicular structure when compared with young spleens. Injured, damaged, aged, or diseased spleens have been shown to exhibit a loss of the marginal zone which separates the germinal center from the red pulp area of the spleen. Injured, damaged, aged, or diseased splenic follicles have been shown to exhibit reduced cell density with areas of low cell-cell contact.

In one aspect of the disclosure, a method is provided for regenerating and/or rejuvenating an injured, damaged, aged, or diseased spleen by administering to an individual (e.g., an aged individual, e.g., having an aged spleen) or to an injured, damaged, aged, or diseased spleen a 15-PGDH inhibitor and/or PGE2 activator in an amount effective to inhibit 15-PGDH activity and/or increase PGE2 activity and/or increase PGE2 synthesis and/or reduce 15-PGDH levels and/or increase PGE2 levels in the individual, thereby regenerating and/or rejuvenating the injured, damaged, aged, or diseased spleen. In some cases, the administering is systemic administration (e.g., orally). In other cases, the administering is local administration (e.g., directly to the spleen). Any suitable administration route as described herein may be used.

In various aspects, after administration of the 15-PGDH inhibitor and/or PGE2 activator, a function of the injured, damaged, aged, or diseased spleen is regenerated and/or rejuvenated relative to a function of the injured, damaged, aged, or diseased spleen prior to treatment with the 15-PGDH inhibitor and/or PGE2 activator. In various aspects, after administration of the 15-PGDH inhibitor and/or PGE2 activator, a function of the injured, damaged, aged, or diseased spleen is rejuvenated to a level substantially similar to a level found in young spleen.

In some embodiments, after administration of the 15-PGDH inhibitor and/or PGE2 activator, the injured, damaged, aged, or diseased spleen exhibits increased or enhanced clearance of pathogens, microorganisms, cellular debris, and/or aged erythrocytes from the blood (e.g., as compared to the injured, damaged, aged, or diseased spleen prior to treatment with the 15-PGDH inhibitor and/or PGE2 activator). In some embodiments, after administration of the 15-PGDH inhibitor and/or PGE2 activator, the injured, damaged, aged, or diseased spleen is capable of clearing pathogens, microorganisms, cellular debris, and/or aged erythrocytes from the blood at levels that are substantially similar to levels found in young spleen. In some embodiments, after administration of the 15-PGDH inhibitor and/or PGE2 activator, the injured, damaged, aged, or diseased spleen exhibits increased lymphoid maturation (e.g., as compared to the injured, damaged, aged, or diseased spleen prior to treatment with the 15-PGDH inhibitor and/or PGE2 activator). In some embodiments, after administration of the 15-PGDH inhibitor and/or PGE2 activator, the injured, damaged, aged, or diseased spleen exhibits levels of lymphoid maturation that are substantially similar to levels found in young spleen.

Regenerating and/or Rejuvenating Injured, Damaged, Aged, or Diseased Epithelial Tissue

The epithelial tissue lines the outer surfaces of organs and blood vessels throughout the body and the inner surfaces of cavities in many internal organs and is comprised of cell types of squamous, cuboidal, columnar, and pseudostratified. Squamous cells decrease friction of a surface for which fluids can move easily. Squamous epithelium can be found lining surfaces such as skin or alveoli in the lung, allowing passive diffusion of molecules or biomolecules. In some cases, squamous epithelium can also form the lining of cavities such as in blood vessels as endothelium, in the pericardium as mesothelium, or in other body cavities. Cuboidal epithelium can be found in secretive tissue such as the exocrine glands, in absorptive tissue such as the pancreas, the lining of the kidney tubules, in the ducts of the glands, covering the female ovary, or lining the walls of the seminiferous tubules in the testes. Cuboidal cells provide protection and can be active in pumping material in or out of the lumen or allowing passive diffusion, depending on location and specialization of the cuboidal cells. Simple cuboidal epithelium can differentiate to form the secretory and duct portions of glands. Stratified cuboidal epithelium protects areas such as the ducts of sweat glands, mammary glands, or salivary glands. Columnar epithelium forms the lining of the stomach and intestines and can possess microvilli for maximizing the surface area for absorption. These microvilli can form a brush border. Other columnar cells can be ciliated to move mucus in the function of mucociliary clearance. Other columnar cells can be ciliated and found in the fallopian tubes, the uterus, or central canal of the spinal cord. Some columnar cells can be specialized for sensory reception such as in the nose, ears, or the taste buds. Hair cells in the inner ears have stereocilia which are similar to microvilli. Goblet cells are modified columnar cells and are found between the columnar epithelial cells of the duodenum for secreting mucus as a lubricant. Single-layered non-ciliated columnar epithelium can indicate an absorptive function. Stratified columnar epithelium can be found in lobar ducts in the salivary glands, the eye, the pharynx, or sex organs. Pseudostratified epithelial cells can be ciliated, where the cilia are capable of energy-dependent pulsatile beating in a certain direction through interaction of cytoskeletal microtubules and connecting structural proteins and enzymes. In the respiratory tract, the wafting effect produced causes mucus secreted locally by the goblet cells (to lubricate and to trap pathogens and particles) to flow in that direction (typically out of the body). Ciliated epithelium can be found in the airways (nose, bronchi), but can also be found in the uterus and Fallopian tubes, where the cilia propel the ovum to the uterus.

The epithelial tissue's main functions include secretion, selective absorption, protection, transcellular transport, and/or sensing. For example, the epithelial tissue provides a protective barrier against mechanical, thermal, and physical injury and hazardous substances, prevents loss of moisture, reduces harmful effects of UV radiation, and/or acts as a sensory organ (touching, detecting temperature, etc). During the aging process, one or more of these functions may be abrogated or reduced. In elderly people, epithelial tissue function has been shown to be decreased or impaired. In addition, aged epithelial tissue has been shown to exhibit abnormal morphology with flattened dermal-epidermal junction, giving the appearance of atrophy and cellular heterogeneity. The melanocyte density declines slowly, and the Langerhans cells decrease in number with advancing age. Among the dermal changes can be attenuation in the number and diameter of elastic fibers in the papillary dermis, an increase in number and thickness of the same fibers in the reticular dermis, and a coarsening of collagen fibers with an increase in density of the collagen network. A decrease in the dermal cell population as well as a functional decline in glandular activity are also noted with intrinsic aging. A decline in hair number, rate of growth, and diameter, along with a slowing of the rate of growth of nails, have also been associated with aging epithelial tissue.

In one aspect of the disclosure, a method is provided for regenerating and/or rejuvenating injured, damaged, aged, or diseased epithelial tissue by administering to an individual (e.g., an aged individual, e.g., having an aged epithelial tissue) or to an injured, damaged, aged, or diseased epithelial tissue a 15-PGDH inhibitor and/or PGE2 activator in an amount effective to inhibit 15-PGDH activity and/or increase PGE2 synthesis and/or increase PGE2 activity and/or reduce 15-PGDH levels and/or increase PGE2 levels in the individual, thereby regenerating and/or rejuvenating the injured, damaged, aged, or diseased epithelial tissue. In some cases, the administering is systemic administration (e.g., orally or intravenously). In other cases, the administering is local administration (e.g., topically or subcutaneously). Any suitable administration route as described herein may be used, including intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

In various aspects, after administration of the 15-PGDH inhibitor and/or PGE2 activator, a function of the injured, damaged, aged, or diseased epithelial tissue is regenerated or rejuvenated relative to a function of the injured, damaged, aged, or diseased epithelial tissue prior to treatment with the 15-PGDH inhibitor and/or PGE2 activator. In various aspects, after administration of the 15-PGDH inhibitor and/or PGE2 activator, a function of the injured, damaged, aged, or diseased epithelial tissue is regenerated or rejuvenated to a level substantially similar to a level found in young epithelial tissue.

In some embodiments, after administration of the 15-PGDH inhibitor and/or PGE2 activator, the injured, damaged, aged, or diseased epithelial tissue exhibits increased or enhanced skin functions such as secretion, selective absorption, protection, transcellular transport, and sensing (e.g., as compared to the injured, damaged, aged, or diseased epithelial tissue prior to treatment with the 15-PGDH inhibitor and/or PGE2 activator) as determined by measuring, e.g., collagen composition or abundance in the epithelial tissue, transepidermal water loss, epithelial tissue hydration (e.g., skin hydration), epithelial tissue dryness (e.g., skin dryness), epithelial tissue elasticity (e.g., skin elasticity), comeocyte adhesion, ceramide concentration, pruritus, water-holding capacity, epithelial skin smoothness and roughness (e.g., skin smoothness and roughness), epithelial tissue wrinkles (e.g., skin wrinkles), epithelial tissue scaling (e.g., skin scaling), epithelial tissue tightness or softness (skin tightness or softness), epithelial tissue reddening and erythema formation (e.g., skin reddening and erythema formation), capillary blood flow, protection of the epithelial tissue against oxidative (including UV-induced or non-UV-induced) damage to nucleic acid, lipid, or protein, depletion of Langerhans cells after UV exposure, delayed-type hypersensitivity immune response to recall antigens in epithelial tissue such as skin or epithelial tissue or skin sebum. In some embodiments, after administration of the 15-PGDH inhibitor and/or PGE2 activator, the injured, damaged, aged, or diseased epithelial tissue is capable of secretion, selective absorption, protection, transcellular transport, and sensing at levels that are substantially similar to levels found in young epithelial tissue. In some embodiments, after administration of the 15-PGDH inhibitor and/or PGE2 activator, the injured, damaged, aged, or diseased epithelial tissue exhibits enhanced skin, increased barrier function, increased hair growth, increased elasticity of skin, and stimulation of hair follicle stem cells.

Regenerating and/or Rejuvenating Injured, Damaged, Aged, or Diseased Vascular Tissue

The vascular tissue is part of an organ system that permits blood to circulate and transport nutrients (such as amino acids and electrolytes), oxygen, carbon dioxide, hormones, or blood cells to and from the cells in the body to provide nourishment, immunity, and homeostasis such as stabilizing temperature or pH. Vascular tissue can include lymphatic system and cardiovascular system and comprises cell types of endothelial cells (ECs) and vascular smooth muscle cells (VSMCs).

The vascular tissue's main functions include transportation and passage of lymph or blood, which includes plasma, red blood cells, white blood cells, and platelets that are circulated by the heart through the vertebrate vascular system, carrying oxygen and nutrients to and waste materials away from all body tissues. Lymph can recycle excess blood plasma after it has been filtered from the interstitial fluid (between cells) and returned to the lymphatic system. During the aging process, one or more of these functions may be abrogated or reduced. In elderly people, vascular tissue function has been shown to be decreased or impaired. In addition, injured, damaged, aged, or diseased vasculature tissue has been shown to exhibit oxidative stress, mitochondrial dysfunction, impaired resistance to molecular stressors, chronic low-grade inflammation, genomic instability, cellular senescence, epigenetic alterations, loss of protein homeostasis, deregulated nutrient sensing, and/or stem cell dysfunction.

In one aspect of the disclosure, a method is provided for regenerating and/or rejuvenating injured, damaged, aged, or diseased vascular tissue by administering to an individual (e.g., an aged individual, e.g., having an injured, damaged, aged, or diseased vascular tissue) or to an injured, damaged, aged, or diseased vascular tissue a 15-PGDH inhibitor and/or PGE2 activator in an amount effective to inhibit 15-PGDH activity and/or increase PGE2 activity and/or increase PGE2 synthesis and/or reduce 15-PGDH levels and/or increase PGE2 levels in the individual, thereby regenerating and/or rejuvenating the injured, damaged, aged, or diseased vascular tissue. In some cases, the administering is systemic administration (e.g., orally or intravenously). In other cases, the administering is local administration (e.g., intramuscularly or intravenously). Any suitable administration route as described herein may be used, including intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

In various aspects, after administration of the 15-PGDH inhibitor and/or PGE2 activator, a function of the injured, damaged, aged, or diseased vascular tissue is rejuvenated relative to a function of the injured, damaged, aged, or diseased vascular tissue prior to treatment with the 15-PGDH inhibitor and/or PGE2 activator. In various aspects, after administration of the 15-PGDH inhibitor and/or PGE2 activator, a function of the injured, damaged, aged, or diseased vascular tissue is regenerated or rejuvenated to a level substantially similar to a level found in young vascular tissue.

In some embodiments, after administration of the 15-PGDH inhibitor and/or PGE2 activator, the injured, damaged, aged, or diseased vascular tissue exhibits increased or enhanced vascular tissue functions (e.g., as compared to the injured, damaged, aged, or diseased vascular tissue prior to treatment with the 15-PGDH inhibitor and/or PGE2 activator) as determined by, e.g., electrocardiography, ultrasound imaging, X-ray computed tomography and positron emission tomography, magnetic resonance imaging, angiography, contrast enhanced ultrasound, optical coherence tomography, flow-sensitive 4D-magnetic resonance imaging, bright field microscopy, fluorescence microscopy, mathematical modeling and abdominal aortic aneurysms, tissue material properties and tensile testing, tissue elasticity imaging, atomic force microscopy, flow cytometry, microfluidics, micropipette aspiration, optical microscopy, optical tweezers, or electron microscopy. In some embodiments, after administration of the 15-PGDH inhibitor and/or PGE2 activator, the injured, damaged, aged, or diseased vascular tissue is capable of functioning at substantially similar to levels found in young vascular tissue.

Regenerating and/or Rejuvenating Injured, Damaged, Aged, or Diseased Brown Fat

Brown fat is adipose tissue characterized by dense mitochondrial concentration along with a high degree of innervation and vascularization. Brown fat is localized in the cervical-supraclavicular region, perirenal and paravertebral region, and around major blood vessels such as the aorta. Brown fat comprises polygonal adipose cells with several liquid cytoplasmic droplets.

Brown fat's main function is to produce heat by non-shivering thermogenesis. Brown fat dissociates oxidative phosphorylation and ATP production due to the presence of an uncoupling protein (e.g., uncoupling protein 1 (UCP1 or thermogenin)) in order to produce heat.

During the aging process, one or more of these functions may be abrogated or reduced. In elderly people, aged brown fat shows decreased mass and activity. In addition, aged brown fat has been shown to display reduced UCP1 activity. Aged brown fat has been shown to display mitochondrial dysfunction and reduced mitochondrial concentration.

Accordingly, in one aspect of the disclosure, a method is provided for regenerating and/or rejuvenating injured, damaged, aged, or diseased brown fat by administering to an individual (e.g., an aged individual, e.g., having injured, damaged, aged, or diseased brown fat) or to injured, damaged, aged, or diseased brown fat a 15-PGDH inhibitor and/or PGE2 activator in an amount effective to inhibit 15-PGDH activity and/or increase PGE2 activity and/or increase PGE2 synthesis and/or reduce 15-PGDH levels and/or increase PGE2 levels in the individual, thereby regenerating and/or rejuvenating the injured, damaged, aged, or diseased brown fat. In some cases, the administering is systemic administration (e.g., orally). In other cases, the administering is local administration (e.g., directly to the brown fat). Any suitable administration route as described herein may be used.

In various aspects, after administration of the 15-PGDH inhibitor and/or PGE2 activator, a function of the injured, damaged, aged, or diseased brown fat is regenerated or rejuvenated relative to a function of the injured, damaged, aged, or diseased brown fat prior to treatment with the 15-PGDH inhibitor and/or PGE2 activator. In various aspects, after administration of the 15-PGDH inhibitor and/or PGE2 activator, a function of the injured, damaged, aged, or diseased brown fat is regenerated or rejuvenated to a level substantially similar to a level found in young brown fat.

In some embodiments, after administration of the 15-PGDH inhibitor and/or PGE2 activator, the injured, damaged, aged, or diseased brown fat exhibits increased volume (e.g., as compared to the injured, damaged, aged, or diseased brown fat prior to treatment with the 15-PGDH inhibitor and/or PGE2 activator) (e.g., as measured by medical imaging techniques, e.g. MRI and fMRI). In some embodiments, after administration of the 15-PGDH inhibitor and/or PGE2 activator, the injured, damaged, aged, or diseased brown fat is present at a volume substantially similar to that of young brown fat. In some embodiments, after administration of the 15-PGDH inhibitor and/or PGE2 activator, the injured, damaged, aged, or diseased brown fat shows increased UCP1 activity (e.g., as compared to the injured, damaged, aged, or diseased brown fat prior to treatment with the 15-PGDH inhibitor and/or PGE2 activator) (e.g., as measured by assaying UCP1 levels in a sample taken from the injured, damaged, aged, or diseased individual). In some embodiments, after administration of the 15-PGDH inhibitor and/or PGE2 activator, the injured, damaged, aged, or diseased brown fat exhibits levels of UCP1 activity that are substantially similar to levels found in young brown fat. In some embodiments, after administration of the 15-PGDH inhibitor and/or PGE2 activator, the injured, damaged, aged, or diseased brown fat exhibits increased mitochondrial concentration or activity (e.g., as compared to the injured, damaged, aged, or diseased brown fat prior to treatment with the 15-PGDH inhibitor and/or PGE2 activator) (e.g., as measured by microscopy performed on a biopsy sample from the injured, damaged, aged, or diseased individual). In some embodiments, after administration of the 15-PGDH inhibitor and/or PGE2 activator, the injured, damaged, aged, or diseased brown fat exhibits a mitochondrial concentration or activity substantially similar to that of young brown fat.

Regenerating and/or Rejuvenating Injured, Damaged, Aged, or Diseased Dental Tissue/Teeth

Teeth comprise several layers of hard and soft tissue. The outermost layer comprises enamel which comprises predominately inorganic minerals (e.g., hydroxyapatite). This outermost layer protects internal layers comprising dentin, cementum, and dental pulp. Dentin comprises hard tissue comprising minerals, organic materials, and water. Cementum comprises hard tissue comprising minerals, organic materials, and water which connects a tooth to the surrounding bone of a jaw. These hard tissues surround the interior dental pulp which comprises soft connective tissue.

During the aging process, changes in the relative abundance and distribution of these tissues take place. Old teeth display a smaller dental pulp chamber and compensatory thickening of the dentin and cementum. Furthermore, receding gums are common in aged individuals. Receding gums can expose the roots of teeth to inflammation and bacterial infection.

Accordingly, in one aspect of the disclosure, a method is provided for regenerating and/or rejuvenating injured, damaged, aged, or diseased teeth and/or gums by administering to an individual (e.g., an injured, damaged, aged, or diseased individual, e.g., having injured, damaged, aged, or diseased teeth) or to injured, damaged, aged, or diseased teeth and/or gums a 15-PGDH inhibitor and/or PGE2 activator in an amount effective to inhibit 15-PGDH activity and/or increase PGE2 activity and/or increase PGE2 synthesis and/or reduce 15-PGDH levels and/or increase PGE2 levels in the individual, thereby regenerating and/or rejuvenating the injured, damaged, aged, or diseased teeth and/or gums. In some cases, the administering is systemic administration (e.g., orally). In other cases, the administering is local topical administration (e.g., directly to the oral cavity or buccal administration). Any suitable administration route as described herein may be used.

In various aspects, after administration of the 15-PGDH inhibitor and/or PGE2 activator, a property of the dental tissue is regenerated or rejuvenated relative to a property of the injured, damaged, aged, or diseased dental tissue prior to treatment with the 15-PGDH inhibitor and/or PGE2 activator. In various aspects, after administration of the 15-PGDH inhibitor and/or PGE2 activator, a property of the injured, damaged, aged, or diseased dental tissue is regenerated or rejuvenated to a level substantially similar to a level found in young dental tissue.

In some embodiments, after administration of the 15-PGDH inhibitor and/or PGE2 activator, the injured, damaged, aged, or diseased dental tissue displays an increased ratio of dentin to dental pulp (e.g., as compared to the injured, damaged, aged, or diseased dental tissue prior to treatment with the 15-PGDH inhibitor and/or PGE2 activator) (e.g., as measured by radiography of dental tissue). In some embodiments, after administration of the 15-PGDH inhibitor and/or PGE2 activator, the injured, damaged, aged, or diseased dental tissue displays a reduced level or reversal of the conversion of dental pulp to dentin compared to untreated injured, damaged, aged, or diseased dental tissue.

Regenerating and/or Rejuvenating Injured, Damaged, Aged, or Diseased Liver

The liver is a central metabolic and endocrine organ. Liver tissue comprises mostly hepatocytes in addition to liver sinusoidal endothelial cells (LSECs), hepatic stellate cells (HSCs) and Kupffer cells (KCs). Hepatocytes are responsible for the majority of hepatic functions.

The liver is responsible for maintaining whole-body homeostasis through regulation of metabolism, xenobiotic, and endobiotic clearance, and molecular biosynthesis. Specific functions of the liver include formation and excretion of bile, regulation of carbohydrate homeostasis, lipid synthesis and secretion of plasma lipid proteins, and control of cholesterol metabolism. The liver is also a central hub of metabolism, participating in the formation of urea, serum albumin, clotting factors, enzymes, and many other proteins.

During the aging process, one or more of these functions may be abrogated or reduced. In elderly people, liver mass and function has been shown to be decreased or impaired. This reduction in liver mass and function can lead to an impaired ability to metabolize many substances in older individuals. Additionally, hepatocytes in elderly individuals display denser body compartments than to hepatocytes in younger subjects.

Accordingly, in one aspect of the disclosure, a method is provided for regenerating and/or rejuvenating injured, damaged, aged, or diseased liver by administering to an individual (e.g., an aged individual, e.g., having an injured, damaged, aged, or diseased liver) or to an injured, damaged, aged, or diseased liver a 15-PGDH inhibitor and/or PGE2 activator in an amount effective to inhibit 15-PGDH activity and/or increase PGE2 activity and/or increase PGE2 synthesis and/or reduce 15-PGDH levels and/or increase PGE2 levels in the individual, thereby regenerating and/or rejuvenating the injured, damaged, aged, or diseased liver. In some cases, the administering is systemic administration (e.g., orally). In other cases, the administering is local administration (e.g., directly to the liver). Any suitable administration route as described herein may be used.

In various aspects, after administration of the 15-PGDH inhibitor and/or PGE2 activator, a function of the injured, damaged, aged, or diseased liver is regenerated or rejuvenated relative to a function of the injured, damaged, aged, or diseased liver prior to treatment with the 15-PGDH inhibitor and/or PGE2 activator. In various aspects, after administration of the 15-PGDH inhibitor and/or PGE2 activator, a function of the injured, damaged, aged, or diseased liver is regenerated or rejuvenated to a level substantially similar to a level found in young liver.

In some embodiments, after administration of the 15-PGDH inhibitor and/or PGE2 activator, the injured, damaged, aged, or diseased liver exhibits increased mass or activity (e.g., as compared to the injured, damaged, aged, or diseased liver prior to treatment with 15-PGDH) (e.g., as measured by liver percussion or medical imaging). In some embodiments, after administration of the 15-PGDH inhibitor and/or PGE2 activator, the injured, damaged, aged, or diseased liver exhibits decreased or reversed reduction in size relative to the injured, damaged, aged, or diseased liver prior to treatment. In some embodiments, after administration of the 15-PGDH inhibitor and/or PGE2 activator, the injured, damaged, aged, or diseased liver exhibits increased xenobiotic clearance (e.g., as compared to the injured, damaged, aged, or diseased liver prior to treatment with the 15-PGDH inhibitor and/or PGE2 activator) (e.g., as measured by administering an exogenous substance to the individual and measuring metabolite formation and/or renal excretion). In some embodiments, after administration of the 15-PGDH inhibitor and/or PGE2 activator, the injured, damaged, aged, or diseased liver exhibits levels of xenobiotic clearance that are substantially similar to levels found in young liver. In some embodiments, after administration of the 15-PGDH inhibitor and/or PGE2 activator, the injured, damaged, aged, or diseased liver exhibits reduced levels of fibrosis.

Regenerating and/or Rejuvenating Injured, Damaged, Aged, or Diseased Hair

Hair is a proteinaceous that grows from the follicles found in the dermis. Hair may be characterized by its color, quantity, and quality. Specifically, hair may be characterized by the presence or absence of pigment (i.e., graying), thickness and curvature, as well as changes in the amount of hair over time (i.e., hair loss).

During the aging process, one or more of these properties may be abrogated or reduced. Changes in hair production may lead to hair loss. Additionally, properties of the hair fiber in aged hair differs from young hair. Aged hair may differ in pigmentation (graying), diameter, curvature, structural properties (stretching, bending, torsional rigidity), and lipid composition.

Accordingly, in one aspect of the disclosure, a method is provided for regenerating and/or rejuvenating injured, damaged, aged, or diseased hair by administering to an individual (e.g., an injured, damaged, aged, or diseased individual, e.g., having injured, damaged, aged, or diseased hair) or to injured, damaged, aged, or diseased hair a 15-PGDH inhibitor and/or PGE2 activator in an amount effective to inhibit 15-PGDH activity and/or increase PGE2 activity and/or increase PGE2 synthesis and/or reduce 15-PGDH levels and/or increase PGE2 levels in the individual, thereby regenerating and/or rejuvenating the injured, damaged, aged, or diseased hair. In some cases, the administering is systemic administration (e.g., orally). In other cases, the administering is topical administration (e.g., directly to the hair or scalp). Any suitable administration route as described herein may be used.

In various aspects, after administration of the 15-PGDH inhibitor and/or PGE2 activator, a property of the injured, damaged, aged, or diseased hair is regenerated or rejuvenated relative to a property of the injured, damaged, aged, or diseased hair prior to treatment with the 15-PGDH inhibitor and/or PGE2 activator. In various aspects, after administration of the 15-PGDH inhibitor and/or PGE2 activator, a property of the injured, damaged, aged, or diseased hair is regenerated or rejuvenated to a level substantially similar to a level found in young hair.

In some embodiments, after administration of the 15-PGDH inhibitor and/or PGE2 activator, the injured, damaged, aged, or diseased hair displays a larger diameter (e.g., as compared to the injured, damaged, aged, or diseased hair prior to treatment with the 15-PGDH inhibitor and/or PGE2 activator) (e.g., as measured by visual inspection, microscopy, or diffraction of a laser). In some embodiments, after administration of the 15-PGDH inhibitor and/or PGE2 activator, the individual exhibits less or no hair loss. In some embodiments, after administration of the 15-PGDH inhibitor and/or PGE2 activator, the injured, damaged, aged, or diseased hair exhibits levels of pigmentation that are substantially similar to young hair.

Regenerating and/or Rejuvenating Injured, Damaged, Aged, or Diseased Small Intestine

The small intestine is a principal organ of the digestive tract. The small intestine comprises three main regions—the duodenum, the jejunum, and the ileum. The duodenum is the shortest and prepares gastric chyme for transit through the rest of the small intestine and absorption through the villi. The jejunum is where products of digestion are principally absorbed through the villi. The ileum is the final section of the small intestine and is where vitamin B12 and bile acids as well as any remaining nutrients are absorbed.

The main function of the small intestine is to absorb nutrients from food. Most of chemical digestion takes place in the small intestine. Once nutrients are degraded into smaller molecules, they are absorbed through the villi which line the length of the small intestine. The small intestine also hosts gut flora which further aid in digestion of certain nutrients and contribute to immunity.

During the aging process, one or more of these functions may be abrogated or reduced. In elderly people, production of the enzyme lactase decreases which can lead to difficulty digesting dairy products. Additionally, changes in the gut flora comprising excessive growth of certain bacteria have been shown to occur with age that lead to difficulty absorbing certain nutrients such as calcium, folic acid, vitamin B12, and iron.

Accordingly, in one aspect of the disclosure, a method is provided for regenerating and/or rejuvenating injured, damaged, aged, or diseased small intestine by administering to an individual (e.g., an aged individual, e.g., having an injured, damaged, aged, or diseased small intestine) or to an injured, damaged, aged, or diseased small intestine a 15-PGDH inhibitor and/or PGE2 activator in an amount effective to inhibit 15-PGDH activity and/or increase PGE2 activity and/or increase PGE2 synthesis and/or reduce 15-PGDH levels and/or increase PGE2 levels in the individual, thereby regenerating and/or rejuvenating the injured, damaged, aged, or diseased small intestine. In some cases, the administering is systemic administration (e.g., orally). In other cases, the administering is local administration (e.g., directly to the small intestine). Any suitable administration route as described herein may be used.

In various aspects, after administration of the 15-PGDH inhibitor and/or PGE2 activator, a function of the injured, damaged, aged, or diseased small intestine is regenerated or rejuvenated relative to a function of the injured, damaged, aged, or diseased small intestine prior to treatment with the 15-PGDH inhibitor and/or PGE2 activator. In various aspects, after administration of the 15-PGDH inhibitor and/or PGE2 activator, a function of the injured, damaged, aged, or diseased small intestine is regenerated or rejuvenated to a level substantially similar to a level found in young small intestine.

In some embodiments, after administration of the 15-PGDH inhibitor and/or PGE2 activator, the injured, damaged, aged, or diseased small intestine exhibits increased production of lactase (e.g., as compared to the injured, damaged, aged, or diseased small intestine prior to treatment with the 15-PGDH inhibitor and/or PGE2 activator) (e.g., as measured by a lactose tolerance test). In some embodiments, after administration of the 15-PGDH inhibitor and/or PGE2 activator, the injured, damaged, aged, or diseased small intestine is capable of producing lactase at levels that are substantially similar to levels found in young small intestine. In some embodiments, after administration of the 15-PGDH inhibitor and/or PGE2 activator, the injured, damaged, aged, or diseased small intestine exhibits reduced growth of certain bacteria (e.g., as compared to the injured, damaged, aged, or diseased small intestine prior to treatment with the 15-PGDH inhibitor and/or PGE2 activator) (e.g., as measured by breath tests, medical imaging, or intestinal fluid culture, or by measuring levels of calcium, iron, folic acid, or vitamin B12 in a sample from the individual). In some embodiments, after administration of the 15-PGDH inhibitor and/or PGE2 activator, the injured, damaged, aged, or diseased small intestine exhibits gut flora substantially similar to that of a young small intestine.

Regenerating and/or Rejuvenating Injured, Damaged, Aged, or Diseased Colon

The colon is the largest component of the large intestine which is the last part of the digestive track. The large intestine comprises the cecum and appendix, the ascending colon, the transverse colon, the descending colon, the sigmoid colon, and the rectum.

The colon's main function is to complete the digestive process by absorbing water and salt from solid wastes before they are eliminated from the body. The movement of contents through the colon is accomplished by peristalsis. Additionally, the colon and large intestine comprise gut flora which can further digest some material not otherwise digested by the digestive track.

During the aging process, one or more of these functions may be abrogated or reduced. In elderly people, the movement of material through the colon via peristalsis has been shown to be decreased. This decrease in peristalsis can lead to constipation and reduce the ability of the colon to perform the function of absorbing water from solid wastes.

Accordingly, in one aspect of the disclosure, a method is provided for regenerating and/or rejuvenating injured, damaged, aged, or diseased colon by administering to an individual (e.g., an aged individual, e.g., having an injured, damaged, aged, or diseased colon) or to an injured, damaged, aged, or diseased colon a 15-PGDH inhibitor and/or PGE2 activator in an amount effective to inhibit 15-PGDH activity and/or increase PGE2 activity and/or increase PGE2 synthesis and/or reduce 15-PGDH levels and/or increase PGE2 levels in the individual, thereby regenerating and/or rejuvenating the injured, damaged, aged, or diseased colon. In some cases, the administering is systemic administration (e.g., orally). In other cases, the administering is local administration (e.g., directly to the colon). Any suitable administration route as described herein may be used.

In various aspects, after administration of the 15-PGDH inhibitor and/or PGE2 activator, a function of the injured, damaged, aged, or diseased colon is regenerated or rejuvenated relative to a function of the injured, damaged, aged, or diseased colon prior to treatment with the 15-PGDH inhibitor and/or PGE2 activator. In various aspects, after administration of the 1-PGDH inhibitor, a function of the injured, damaged, aged, or diseased colon is regenerated or rejuvenated to a level substantially similar to a level found in young colon.

In some embodiments, after administration of the 15-PGDH inhibitor and/or PGE2 activator, the injured, damaged, aged, or diseased colon exhibits increased or enhanced peristalsis (e.g., as compared to the injured, damaged, aged, or diseased colon prior to treatment with the 15-PGDH inhibitor and/or PGE2 activator) (e.g., as measured by a physician during a general physical exam, a digital rectal exam, blood tests, x-ray, colonic transit study, colonoscopy, sigmoidoscopy, or the like). In some embodiments, after administration of the 15-PGDSH inhibitor, the injured, damaged, aged, or diseased colon is exhibits peristalsis at levels that are substantially similar to a young colon.

Regenerating and/or Rejuvenating Injured, Damaged, Aged, or Diseased Ovaries and Other Reproductive Tissues

Ovaries comprise the female gonads. The outer layer of the ovaries is the ovarian cortex comprising ovarian follicles suspended in a matrix of stromal cells. In addition to the ovaries, key reproductive tissues include the oviduct and the uterus. The oviduct is the site for transport of oocytes and the platform for fertilization and early embryo development. The uterus is the essential organ for pregnancy that provides the structure for the development of an embryo.

The main function of the ovaries are the produce and release oocytes. The ovaries are also involved in the production and secretion of hormones such as estrogen, androgen, inhibin, and progesterone and therefore in the regulation of pregnancy and secondary sex characteristics. The oviduct and uterus are also involved in the development of an embryo throughout pregnancy.

During the aging process, one or more of these functions may be abrogated or reduced. In elderly people, a decline in the number of ovary follicles leads to a decline in the performance of the ovaries comprising a decrease in the number of oocytes. In addition, there is a parallel increase in pregnancy failure and chromosomally aberrant conceptions with increasing age.

Accordingly, in one aspect of the present disclosure, a method is provided for regenerating and/or rejuvenating injured, damaged, aged, or diseased ovaries and other reproductive tissues by administering to an individual (e.g., an aged individual, e.g., having injured, damaged, aged, or diseased ovaries or other reproductive tissues) or to injured, damaged, aged, or diseased ovaries or other reproductive tissues a 15-PGDH inhibitor and/or PGE2 activator in an amount effective to inhibit 15-PGDH activity and/or increase PGE2 activity and/or increase PGE2 synthesis and/or reduce 15-PGDH levels and/or increase PGE2 levels in the individual, thereby regenerating and/or rejuvenating the injured, damaged, aged, or diseased ovaries or other reproductive tissues. In some cases, the administering is system administration (e.g., orally). In other cases, the administering is local administration (e.g., directly to the reproductive tissues). Any suitable administration route as described herein may be used.

In various aspects, after administration of the 15-PGDH inhibitor and/or PGE2 activator, the injured, damaged, aged, or diseased ovaries or other reproductive tissues is regenerated or rejuvenated relative to a function of the injured, damaged, aged, or diseased ovaries or other reproductive tissues prior to treatment with the 15-PGDH inhibitor and/or PGE2 activator. In various aspects, after administration of the 15-PGDH inhibitor and/or PGE2 activator, a function of the injured, damaged, aged, or diseased ovaries or other reproductive tissues is regenerated or rejuvenated to a level substantially similar to a level found in young reproductive tissues.

In some embodiments, after administration of the 15-PGDH inhibitor and/or PGE2 activator, the injured, damaged, aged, or diseased reproductive tissues exhibit reduced or halted ovary decline (e.g., as compared to the injured, damaged, aged, or diseased reproductive tissues prior to treatment with the 15-PGDH) (e.g., as measured by blood or urine tests for hormone levels or egg reserves or imaging tests and procedures such as ultrasound exam, sonohysterography, hysterosalpingoraphy, hysteroscopy, or laparoscopy). In some embodiments, after administration of the 15-PGDH inhibitor and/or PGE2 activator, the injured, damaged, aged, or diseased ovaries show stopped or reduced decline in the number of ovary follicles or ovary follicles substantially similar to young ovaries. In some embodiments, after administration of the 15-PGDH inhibitor and/or PGE2 activator, the individual exhibits a reduction in pregnancy failure and/or number of chromosomally aberrant conceptions (e.g., as compared to the individual prior to treatment with the 15-PGDH inhibitor and/or PGE2 activator) (e.g., as measured by amniocentesis or chorionic villus sampling). In some embodiments, after administration of the 15-PGDH inhibitor and/or PGE2 activator, the individual exhibits a rate of pregnancy failure and/or chromosomally aberrant conceptions that is substantially similar to that of an individual with young ovaries and other reproductive tissues.

Regenerating and/or Rejuvenating Injured, Damaged, Aged, or Diseased Skin/Epidermal Tissue

The epidermal tissue (or skin) serves as a barrier to protect the body against microbial pathogens, oxidant stress (UV light), and chemical compounds, and provides mechanical resistance to minor injury. The epidermal tissue can contain nerve receptors that allow a subject to feel touch, pain, and pressure, help control fluid and electrolyte balance, help control body temperature, and/or protect a subject from the environment. The epidermal tissue can be divided into three main parts. The outer part (or epidermis) can contain skin cells, pigment, and proteins. The middle part (or dermis) contains skin cells, blood vessels, nerves, hair follicles, and oil glands. The dermis can provide nutrients to the epidermis. The inner layer under the dermis (or the subcutaneous layer) can contain sweat glands, some hair follicles, blood vessels, and fat. Each layer can also contain connective tissue with collagen fibers to give support and elastin fibers to provide flexibility and strength.

With aging, the outer skin layer (epidermis) thins. The number of cell layers may remain unchanged. The number of pigment-containing cells (melanocytes) can decrease. The remaining melanocytes can increase in size. Aging skin may look thinner, paler, and clear (translucent). Pigmented spots including age spots or “liver spots” may appear in sun-exposed areas. The medical term for these areas is lentigos. Changes in the connective tissue can reduce the skin's strength and elasticity (known as elastosis). The blood vessels of the dermis can become more fragile, which may lead to bruising, bleeding under the skin (often called senile purpura), cherry angiomas, and similar conditions. Sebaceous glands can produce less oil as one ages. This can make it harder to keep the skin moist, resulting in dryness and itchiness. The subcutaneous fat layer may thin so it has less insulation and padding, which may increase risk of skin injury and reduce ability to maintain body temperature. Some medicines may be absorbed by the fat layer. Shrinkage of this layer may change the way that these medicines work. The sweat glands can produce less sweat during aging, which can increase risk for overheating or developing heat stroke. Growths such as skin tags, warts, brown rough patches (seborrheic keratoses), and other blemishes can be common in older people.

Methods are provided herein to rejuvenate an injured, damaged, aged, or diseased epidermal tissue. In one aspect of the disclosure, a method is provided for regenerating and/or rejuvenating injured, damaged, aged, or diseased skin by administering to an individual (e.g., an aged individual, e.g., having injured, damaged, aged, or diseased skin) or to an injured, damaged, aged, or diseased skin a 15-PGDH inhibitor and/or PGE2 activator in an amount effective to inhibit 15-PGDH activity and/or increase PGE2 activity and/or increase PGE2 synthesis and/or reduce 15-PGDH levels and/or increase PGE2 levels in the individual, thereby regenerating and/or rejuvenating the injured, damaged, aged, or diseased skin. In some cases, the administering is systemic administration (e.g., orally). In other cases, the administering is local administration (e.g., directly to the skin, e.g., by topical administration). In some cases, the administering is topical administration or intradermal injection. Any suitable administration route as described herein may be used.

In various aspects, after administration of the 15-PGDH inhibitor and/or PGE2 activator, a function of the injured, damaged, aged, or diseased epidermal tissue is regenerated or rejuvenated relative to a function of the injured, damaged, aged, or diseased epidermal tissue prior to treatment with the 15-PGDH inhibitor and/or PGE2 activator. In various aspects, after administration of the 15-PGDH inhibitor and/or PGE2 activator, a function of the injured, damaged, aged, or diseased epidermal tissue is regenerated or rejuvenated to a level substantially similar to a level found in young epidermal tissue.

In some embodiments, after administration of the 15-PGDH inhibitor and/or PGE2 activator, the injured, damaged, aged, or diseased epidermal tissue exhibits enhanced skin condition. In some embodiments, after administration of the 15-PGDH inhibitor and/or PGE2 activator, the injured, damaged, aged, or diseased epidermal tissue exhibits increased barrier function. In some embodiments, after administration of the 15-PGDH inhibitor and/or PGE2 activator, the injured, damaged, aged, or diseased epidermal tissue supports increased hair growth. The administration of the 15-PGDH inhibitor and/or PGE2 activator can counter baldness. The administration of the 15-PGDH inhibitor and/or PGE2 activator can stimulate hair follicle stem cells. In some embodiments, after administration of the 15-PGDH inhibitor and/or PGE2 activator, the injured, damaged, aged, or diseased epidermal tissue exhibits increased elasticity of skin.

Regenerating and/or Rejuvenating Injured, Damaged, Aged, or Diseased Brain

The brain is made up of several functional parts, each with a specific and important function. The functional parts can include frontal lobe, temporal lobe, parietal lobe, occipital lobe, cerebellum and brain stem. The brain can control our ability to balance, walk, talk, eat, process information, make decision and feel emotions. The brain can coordinate and regulate breathing, blood circulation, hormone release, and heart rate.

Ageing can cause changes to the brain size, vasculature, and cognition. The brain may shrink with increasing age and there may be changes at all levels from molecules to morphology. Incidence of stroke, white matter lesions, dementia and level of memory impairment can also rise with age. There may be changes in levels of neurotransmitters and hormones during aging. Ageing may have its effects on the molecules, cells, vasculature, gross morphology, and cognition. In some cases, ageing can cause physical changes on the brain. The shrinking of grey matter may due to neuronal cell death. Declining in neuronal volume rather than number may contribute to the changes in an ageing brain and that it may be related to sex with different areas most affected in men and women. There may be changes in dendritic arbor, spines, or synapses. In some cases, aging can cause cognitive change on the brain. Cognitive change associated with ageing can be that of memory. Memory function can be broadly divided into four sections, episodic memory, semantic memory, procedural memory, and working memory. Episodic memory performance may decline from middle age onwards, and may also be a characteristic of the memory loss seen in Alzheimer's disease (AD). Levels of neurotransmitters (e.g., dopamine and serotonin) may fall as brain ages. Other factors that have been implicated in the ageing brain include calcium dysregulation, mitochondrial dysfunction, and the production of reactive oxygen species. Hormone (e.g., sex hormone and growth hormone) levels may also decline as brain ages.

Methods are provided herein to rejuvenate an injured, damaged, aged, or diseased brain. In one aspect of the disclosure, a method is provided for regenerating and/or rejuvenating injured, damaged, aged, or diseased brain by administering to an individual (e.g., an aged individual, e.g., having an injured, damaged, aged, or diseased brain) or to an injured, damaged, aged, or diseased brain a 15-PGDH inhibitor and/or PGE2 activator in an amount effective to inhibit 15-PGDH activity and/or increase PGE2 activity and/or increase PGE2 synthesis and/or reduce 15-PGDH levels and/or increase PGE2 levels in the individual, thereby regenerating and/or rejuvenating the injured, damaged, aged, or diseased brain. In some cases, the administering is systemic administration (e.g., orally). In other cases, the administering is local administration (e.g., directly to the brain). In some cases, the administering is intrathecal administration. In some cases, the administering is intracerebroventricular (ICV) administration. Any suitable administration route as described herein may be used.

In various aspects, after administration of the 15-PGDH inhibitor and/or PGE2 activator, a function of the injured, damaged, aged, or diseased brain is regenerated or rejuvenated relative to a function of the injured, damaged, aged, or diseased brain prior to treatment with the 15-PGDH inhibitor and/or PGE2 activator. In various aspects, after administration of the 15-PGDH inhibitor and/or PGE2 activator, a function of the injured, damaged, aged, or diseased brain is regenerated or rejuvenated to a level substantially similar to a level found in young brain.

In some embodiments, after administration of the 15-PGDH inhibitor and/or PGE2 activator, the injured, damaged, aged, or diseased brain exhibits increased brain size. For example, the injured, damaged, aged, or diseased brain may exhibit increased grey matter, increased amount of neuronal cells, or increased neuronal volume after administration. In some embodiments, after administration of the 15-PGDH inhibitor and/or PGE2 activator, the injured, damaged, aged, or diseased brain exhibits improved cognitive performance. For example, the injured, damaged, aged, or diseased brain may exhibit improved memory performance after administration. In some embodiments, after administration of the 15-PGDH inhibitor and/or PGE2 activator, the injured, damaged, aged, or diseased brain exhibits increased level of neurotransmitters such as dopamine, serotonin and other brain-derived neurotrophic factors. In some embodiments, after administration of the 15-PGDH inhibitor and/or PGE2 activator, the injured, damaged, aged, or diseased brain exhibits increased level of hormones (e.g., oestrogen and growth hormone). In some embodiments, after administration of the 15-PGDH inhibitor and/or PGE2 activator, the injured, damaged, aged, or diseased brain exhibits reduced risk of stroke, white matter lesions, or dementia. In some embodiments, after administration of the 15-PGDH inhibitor and/or PGE2 activator, the injured, damaged, aged, or diseased brain exhibits reduced risk of Alzheimer's or Parkinson's disease. In some embodiments, after administration of the 15-PGDH inhibitor and/or PGE2 activator, the injured, damaged, aged, or diseased brain of a subject having a Alzheimer's or Parkinson's disease may be prevented from worsening. In some embodiments, after administration of the 15-PGDH inhibitor and/or PGE2 activator, cognitive performance of the injured, damaged, aged, or diseased brain of a subject having a Alzheimer's or Parkinson's disease may be restored. For example, plaques or tangles associated with Alzheimer's disease may be reduced or may be inhibited from further developing.

Regenerating and/or Rejuvenating Injured, Damaged, Aged, or Diseased Heart and/or Cardiac Muscle

The cardiac muscle is a specialized type of muscle tissue that forms the heart. This muscle tissue, which contracts and releases involuntarily, is responsible for keeping the heart pumping blood around the body. Cardiac muscle comprises cardiac muscle cells or cardiomyocytes (also known as myocardiocytes or cardiac myocytes are the muscle cells (myocytes) that make up the cardiac muscle (heart muscle). Each cardiac muscle cell contains myofibrils, which are specialized organelles consisting of long chains of sarcomeres, the fundamental contractile units of muscle cells.

Cardiomyocytes show striations similar to those on skeletal muscle cells. Cardiomyocytes have a high mitochondrial density, which allows them to produce adenosine triphosphate (ATP) quickly, making them highly resistant to fatigue. The cardiac muscle's main function includes pumping the blood, which includes plasma, red blood cells, white blood cells, and platelets that is circulated by the heart through the vertebrate vascular system, carrying oxygen and nutrients to and waste materials away from all body tissues. During the aging process, one or more of these functions (e.g., pumping oxygenated blood to the other body parts; pumping hormones and other vital substances to different parts of the body; receiving deoxygenated blood and carrying metabolic waste products from the body and pumping it to the lungs for oxygenation; or maintaining blood pressure) can be abrogated or reduced. In elderly people, cardiac muscle function has been shown to be decreased or impaired. In some cases, the injured, damaged, aged, or diseased cardiac muscle has been shown to exhibit similar impairment as vascular tissue such as oxidative stress, mitochondrial dysfunction, impaired resistance to molecular stressors, chronic low-grade inflammation, genomic instability, cellular senescence, epigenetic alterations, loss of protein homeostasis, deregulated nutrient sensing, or stem cell dysfunction. In some cases, the injured, damaged, aged, or diseased cardiac muscle can impact heart function and anatomy. The heart has a natural pacemaker system that controls the heartbeat. Some of the pathways of this system can develop fibrous tissue and fat deposits due to aging. The natural pacemaker (the sinoatrial or SA node) loses some of its cells. These changes can result in a slightly slower heart rate. Aging can also lead to increase in the size of the heart, especially the left ventricle. The heart wall thickens, so the amount of blood that the chamber can hold may actually decrease despite the increased overall heart size. The heart can fill more slowly. Heart changes often cause the electrocardiogram (ECG) of a normal, healthy older person to be slightly different than the ECG of a healthy younger adult. Abnormal rhythms (arrhythmias), such as atrial fibrillation, are more common in aging cardiac muscle. They may be caused by several types of heart disease. Normal changes in the heart include deposits of the “aging pigment,” lipofuscin. The heart muscle cells degenerate slightly. The valves inside the heart, which control the direction of blood flow, thicken and become stiffer. A heart murmur caused by valve stiffness can be common in aging heart.

Accordingly, in one aspect of the disclosure, a method is provided for regenerating and/or rejuvenating injured, damaged, aged, or diseased cardiac muscle by administering to an individual (e.g., an aged individual, e.g., having an injured, damaged, aged, or diseased cardiac muscle) or to an injured, damaged, aged, or diseased cardiac muscle a 15-PGDH inhibitor and/or PGE2 activator in an amount effective to inhibit 15-PGDH activity and/or increase PGE2 activity and/or increase PGE2 synthesis and/or reduce 15-PGDH levels and/or PGE2 levels in the individual, thereby regenerating and/or rejuvenating the injured, damaged, aged, or diseased cardiac muscle. In some cases, the administering is systemic administration (e.g., orally or intravenously). In other cases, the administering is local administration (e.g., intraventricularly). Any suitable administration route as described herein may be used, including intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

In various aspects, after administration of the 15-PGDH inhibitor and/or PGE2 activator, a function of the injured, damaged, aged, or diseased cardiac muscle is regenerated or rejuvenated relative to a function of the injured, damaged, aged, or diseased cardiac muscle prior to treatment with the 15-PGDH inhibitor and/or PGE2 activator. In various aspects, after administration of the 15-PGDH inhibitor and/or PGE2 activator, a function of the injured, damaged, aged, or diseased cardiac muscle is regenerated or rejuvenated to a level substantially similar to a level found in young cardiac muscle.

In some embodiments, after administration of the 15-PGDH inhibitor and/or PGE2 activator, the injured, damaged, aged, or diseased cardiac muscle exhibits increased or enhanced cardiac muscle tissue functions (e.g., as compared to the injured, damaged, aged, or diseased cardiac muscle prior to treatment with the 15-PGDH inhibitor and/or PGE2 activator) as determined by: echocardiogram, transesophageal echocardiography (TEE), electrocardiogram (ECG or EKG), magnetic resonance imaging (MRI), CT scan, exercise cardiac stress test, pharmacologic stress test, tilt test, ambulatory rhythm monitoring tests, or coronary angiogram. In some embodiments, after administration of the 15-PGDH inhibitor and/or PGE2 activator, the injured, damaged, aged, or diseased cardiac muscle is capable of functioning at substantially similar to levels found in young cardiac muscle. In some embodiments, after administration of the 15-PGDH inhibitor and/or PGE2 activator, the injured, damaged, aged, or diseased cardiac muscle is capable of functioning such as pumping oxygenated blood to the other body parts; pumping hormones and other vital substances to different parts of the body; receiving deoxygenated blood and carrying metabolic waste products from the body and pumping it to the lungs for oxygenation; or maintaining blood pressure at levels that are substantially similar to levels found in young cardiac muscle. In some embodiments, after administration of the 15-PGDH inhibitor and/or PGE2 activator, the injured, damaged, aged, or diseased cardiac muscle exhibits decreased fibrosis or exhibits a fibrosis level that is substantially similar to a fibrosis level in young cardiac muscle.

Regenerating and/or Rejuvenating Injured, Damaged, Aged, or Diseased Bone and/or Injured, Damaged, Aged, or Diseased Cartilage

Bone is a rigid tissue that constitutes part of the vertebrate skeleton. Bones protect the various organs of the body, produce red and white blood cells, store minerals, provide structure and support for the body, and enable mobility. Bone tissue (osseous tissue) is a hard tissue, a type of specialized connective tissue and made up of different types of bone cells. Osteoblasts and osteocytes are involved in the formation and mineralization of bone: osteoclasts are involved in the resorption of bone tissue. Modified (flattened) osteoblasts become the lining cells that form a protective layer on the bone surface. The mineralized matrix of bone tissue has an organic component of mainly collagen called ossein and an inorganic component of bone mineral made up of various salts. Other types of tissue found in bones include bone marrow, endosteum, periosteum, nerves, blood vessels and cartilage.

Cartilage is a resilient and smooth elastic tissue, rubber-like padding that covers and protects the ends of long bones at the joints and nerves, and is a structural component of the rib cage, the ear, the nose, the bronchial tubes, the intervertebral discs, and other body components. The matrix of cartilage is made up of glycosaminoglycans, proteoglycans, collagen fibers, or elastin. Cartilage is composed of specialized cells called chondrocytes that produce a large amount of collagenous extracellular matrix, abundant ground substance that is rich in proteoglycan and elastin fibers. Cartilage is classified in three types, elastic cartilage, hyaline cartilage and fibrocartilage, which differ in relative amounts of collagen and proteoglycan.

The bone and cartilage's main function includes mechanical support and movement; hematopoiesis; storage of mineral or fat, stabilizing pH or calcium; hormone secretion: lubrication; or damages repair. During the aging process, one or more of these functions may be abrogated or reduced. In elderly people, bone and cartilage function has been shown to be decreased or impaired. In addition, injured, damaged, aged, or diseased bone and cartilage can lead to osteomalacia, osteoporosis, or osteoarthritis.

Accordingly, in one aspect of the disclosure, a method is provided for regenerating and/or rejuvenating injured, damaged, aged, or diseased bone and/or injured, damaged, aged, or diseased cartilage by administering to an individual (e.g., an aged individual, e.g., having an injured, damaged, aged, or diseased bone and/or an injured, damaged, aged, or diseased cartilage) or to an injured, damaged, aged, or diseased bone and/or injured, damaged, aged, or diseased cartilage a 15-PGDH inhibitor and/or PGE2 activator in an amount effective to inhibit 15-PGDH activity and/or increase PGE2 activity and/or increase PGE2 synthesis and/or reduce 15-PGDH levels and/or increase PGE2 levels in the individual, thereby regenerating and/or rejuvenating the injured, damaged, aged, or diseased bone and/or injured, damaged, aged, or diseased cartilage. In some cases, the administering is systemic administration (e.g., orally or intravenously). In other cases, the administering is local administration (e.g., intraosseous infusion). Any suitable administration route as described herein may be used, including intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

In various aspects, after administration of the 15-PGDH inhibitor and/or PGE2 activator, a function of the injured, damaged, aged, or diseased bone and/or injured, damaged, aged, or diseased cartilage is regenerated or rejuvenated relative to a function of the injured, damaged, aged, or diseased bone and/or injured, damaged, aged, or diseased cartilage prior to treatment with the 15-PGDH inhibitor and/or PGE2 activator. In various aspects, after administration of the 15-PGDH inhibitor and/or PGE2 activator, a function of the injured, damaged, aged, or diseased bone and/or injured, damaged, aged, or diseased cartilage is regenerated or rejuvenated to a level substantially similar to a level found in young bone and/or young cartilage.

In some embodiments, after administration of the 15-PGDH inhibitor and/or PGE2 activator, the injured, damaged, aged, or diseased bone and/or injured, damaged, aged, or diseased cartilage exhibits increased or enhanced bone and/or cartilage tissue functions (e.g., as compared to the injured, damaged, aged, or diseased bone and/or injured, damaged, aged, or diseased cartilage prior to treatment with the 15-PGDH inhibitor and/or PGE2 activator) as determined by: mechanical methods (e.g., whole-bone mechanical testing, bulk tissue specimen mechanical testing, microbeam mechanical testing, microindentation, or nanoindentation); imaging methods (e.g., computerized tomography (CT), magnetic resonance imaging (MRI), nuclear magnetic resonance (NMR), fourier-transform infrared spectroscopy (FTIR), Raman imaging, or scanning electron microscopy); chemical or physical methods (gravimetric analysis or chemical analysis of collagen crosslinks); bone densitometry.

In some embodiments, after administration of the 15-PGDH inhibitor and/or PGE2 activator, the injured, damaged, aged, or diseased bone and/or injured, damaged, aged, or diseased cartilage is capable of functioning at substantially similar to levels found in young bone and/or cartilage. In some embodiments, after administration of the 15-PGDH inhibitor and/or PGE2 activator, the injured, damaged, aged, or diseased bone and/or injured, damaged, aged, or diseased cartilage is capable of functioning such as mechanical support and movement; hematopoiesis; storage of mineral or fat; stabilizing pH or calcium; hormone secretion: lubrication; or damages repair at levels that are substantially similar to levels of function found in young bone and/or cartilage. In some embodiments, after administration of the 15-PGDH inhibitor and/or PGE2 activator, the injured, damaged, aged, or diseased bone and/or injured, damaged, aged, or diseased cartilage exhibits decreased fibrosis or exhibits a fibrosis level that is substantially similar to a fibrosis level in young bone and/or cartilage. In some embodiments, after administration of the 15-PGDH inhibitor and/or PGE2 activator, the injured, damaged, aged, or diseased bone and/or injured, damaged, aged, or diseased cartilage exhibits increased growth of bone or cartilage. In some embodiments, after administration of the 15-PGDH inhibitor and/or PGE2 activator, the injured, damaged, aged, or diseased cartilage exhibits increased proteoglycan levels or exhibits a level of proteoglycans that is substantially similar to a proteoglycan level in young cartilage. In some embodiments, after administration of the 15-PGDH inhibitor and/or PGE2 activator, the injured, damaged, aged, or diseased tendon or ligament exhibits a level of extracellular matrix (ECM) proteins that is substantially similar to an ECM protein level in young tendon or ligament.

Regenerating and/or Rejuvenating Injured, Damaged, Aged, or Diseased Sensory Organs (e.g., Eye, Ear, Nose, Tongue)

Sensory organs are organs that sense and transduce stimuli and can include eyes, ears, skin, nose, or mouth that correspond to a respective visual system (sense of vision), auditory system (sense of hearing), somatosensory system (sense of touch), olfactory system (sense of smell), and gustatory system (sense of taste). In some cases, the sensory organs are for sensing internal sensation, or interoception, for detecting stimuli from internal organs and tissues. Such sensory organs can include vestibular system (sense of balance) sensed by the inner ear and providing the perception of spatial orientation: proprioception (body position); and nociception (pain). Additionally, sensory organ can include internal chemoreception or and osmoreception-based sensory systems, leading to various perceptions such as hunger, thirst, suffocation, nausea, or different involuntary behaviors, such as vomiting.

The sensory organ's main function includes sensing stimuli such as physical stimuli such as pressure and vibration, sensation of sound, or body position (balance); light (visible electromagnetic radiation); chemical stimuli such as taste or smell: pain: temperature; or other internal stimuli. During the aging process, one or more of these functions may be abrogated or reduced. In elderly people, sensory organ function has been shown to be decreased or impaired.

Accordingly, in one aspect of the disclosure, a method is provided for regenerating and/or rejuvenating injured, damaged, aged, or diseased sensory organ by administering to an individual (e.g., an aged individual, e.g., having an injured, damaged, aged, or diseased sensory organ) or to an injured, damaged, aged, or diseased sensory organ a 15-PGDH inhibitor and/or PGE2 activator in an amount effective to inhibit 15-PGDH activity and/or increase PGE2 activity and/or increase PGE2 synthesis and/or reduce 15-PGDH levels and/or increase PGE2 levels in the individual, thereby regenerating and/or rejuvenating the injured, damaged, aged, or diseased sensory organ. In some cases, the administering is systemic administration (e.g., orally or intravenously). In other cases, the administering is local administration (e.g., intraocularly or intranasally). Any suitable administration route as described herein may be used, including intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

In various aspects, after administration of the 15-PGDH inhibitor and/or PGE2 activator, a function of the injured, damaged, aged, or diseased sensory organ is regenerated or rejuvenated relative to a function of the injured, damaged, aged, or diseased sensory organ prior to treatment with the 15-PGDH inhibitor and/or PGE2 activator. In various aspects, after administration of the 15-PGDH inhibitor and/or PGE2 activator, a function of the injured, damaged, aged, or diseased sensory organ is regenerated or rejuvenated to a level substantially similar to a level found in young sensory organ.

In some embodiments, after administration of the 15-PGDH inhibitor and/or PGE2 activator, the injured, damaged, aged, or diseased sensory organ exhibits increased or enhanced sensory organ tissue functions (e.g., as compared to the injured, damaged, aged, or diseased sensory organ prior to treatment with the 15-PGDH inhibitor and/or PGE2 activator) as determined by: visual test, hearing test, olfactory test, physical fitness or balance test, or any other tests that examine internal or external senses or stimuli.

In some embodiments, after administration of the 15-PGDH inhibitor and/or PGE2 activator, the injured, damaged, aged, or diseased sensory organ is capable of functioning at substantially similar to levels found in young sensory organ. In some embodiments, after administration of the 15-PGDH inhibitor and/or PGE2 activator, the injured, damaged, aged, or diseased sensory organ is capable of functioning such as sensing stimuli such as physical stimuli such as pressure and vibration, sensation of sound, or body position (balance); light (visible electromagnetic radiation): chemical stimuli such as taste or smell; pain; temperature; or other internal stimuli at levels that are substantially similar to levels of function found in young sensory organ. In some embodiments, after administration of the 15-PGDH inhibitor and/or PGE2 activator, the injured, damaged, aged, or diseased eye exhibits decreased level of dry eye disease, lacrimal gland inflammation, or macular degeneration

Regenerating and/or Rejuvenating Injured, Damaged, Aged, or Diseased Kidney

Kidney is an organ that receives blood from renal arteries, and the blood exits into renal veins. Kidney is also connected to a ureter, which carries urine to the bladder. Exemplary cell types of kidney include glomerulus parietal cell, glomerulus podocyte, proximal tubule brush border cell. Loop of Henle thin segment cell, thick ascending limb cell, distal tubule cell, collecting duct principal cell, collecting duct intercalated cell, or interstitial kidney cells. The kidney participates in the control of the volume of various body fluids, fluid osmolality, acid-base balance, various electrolyte concentrations, and removal of toxins. Filtration occurs in the glomerulus: one-fifth of the blood volume that enters the kidneys is filtered. Examples of substances reabsorbed are solute-free water, sodium, bicarbonate, glucose, and amino acids.

Examples of substances secreted are hydrogen, ammonium, potassium and uric acid. The kidney also carries out functions independent of the nephron. For example, they convert a precursor of vitamin D to its active form, calcitriol; and synthesize the hormones erythropoietin and renin.

The kidney's main function includes formation of urine (e.g., filtration, reabsorption, secretion, or excretion); hormone secretion; blood pressure regulation; acid-base balance; or regulation of osmolality. During the aging process, one or more of these functions may be abrogated or reduced. In elderly people, kidney function has been shown to be decreased or impaired.

Accordingly, in one aspect of the disclosure, a method is provided for regenerating and/or rejuvenating injured, damaged, aged, or diseased kidney by administering to an individual (e.g., an aged individual, e.g., having an injured, damaged, aged, or diseased kidney) or to an injured, damaged, aged, or diseased kidney a 15-PGDH inhibitor and/or PGE2 activator in an amount effective to inhibit 15-PGDH activity and/or increase PGE2 activity and/or increase PGE2 synthesis and/or reduce 15-PGDH levels and/or increase PGE2 levels in the individual, thereby regenerating and/or rejuvenating the injured, damaged, aged, or diseased kidney. In some cases, the administering is systemic administration (e.g., orally). In other cases, the administering is local administration (e.g., intravenously or subcutaneously). Any suitable administration route as described herein may be used, including intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

In various aspects, after administration of the 15-PGDH inhibitor and/or PGE2 activator, a function of the injured, damaged, aged, or diseased kidney is regenerated or rejuvenated relative to a function of the injured, damaged, aged, or diseased kidney prior to treatment with the 15-PGDH inhibitor and/or PGE2 activator. In various aspects, after administration of the 15-PGDH inhibitor and/or PGE2 activator, a function of the injured, damaged, aged, or diseased kidney is regenerated or rejuvenated to a level substantially similar to a level found in young kidney.

In some embodiments, after administration of the 15-PGDH inhibitor and/or PGE2 activator, the injured, damaged, aged, or diseased kidney exhibits increased or enhanced kidney tissue functions (e.g., as compared to the injured, damaged, aged, or diseased kidney prior to treatment with the 15-PGDH inhibitor and/or PGE2 activator) as determined by: clinical assessment; urine tests; blood tests (e.g., glomerular filtration rate); medical imaging (e.g., CT scan); or biopsy.

In some embodiments, after administration of the 15-PGDH inhibitor and/or PGE2 activator, the injured, damaged, aged, or diseased kidney is capable of functioning at substantially similar to levels found in young kidney. In some embodiments, after administration of the 15-PGDH inhibitor and/or PGE2 activator, the injured, damaged, aged, or diseased kidney is capable of functioning such as formation of urine (e.g., filtration, reabsorption, secretion, or excretion); hormone secretion; blood pressure regulation; acid-base balance; or regulation of osmolality at levels that are substantially similar to levels of function found in young kidney. In some embodiments, after administration of the 15-PGDH inhibitor and/or PGE2 activator, the injured, damaged, aged, or diseased kidney exhibits decreased level of kidney disease such as chronic kidney disease, nephritic and nephrotic syndromes, acute kidney injury, pyelonephritis, or kidney cancer.

Regenerating and/or Rejuvenating Injured, Damaged, Aged, or Diseased Thyroid

Thyroid is an endocrine gland that comprises spherical thyroid follicle, lined with follicular cells (thyrocytes), and parafollicular cells that surround a lumen containing colloid. The thyroid gland secretes three hormones: the two thyroid hormones: triiodothyronine (T3) and thyroxine (T4); and a peptide hormone, calcitonin. The thyroid hormones influence the metabolic rate and protein synthesis. Calcitonin plays a role in calcium homeostasis. The thyroid's main function includes regulating, producing, and secreting hormones. During the aging process, one or more of these functions may be abrogated or reduced. In elderly people, thyroid function has been shown to be decreased or impaired.

Accordingly, in one aspect of the disclosure, a method is provided for regenerating and/or rejuvenating injured, damaged, aged, or diseased thyroid by administering to an individual (e.g., an aged individual, e.g., having an injured, damaged, aged, or diseased thyroid) or to an injured, damaged, aged, or diseased thyroid a 15-PGDH inhibitor and/or PGE2 activator in an amount effective to inhibit 15-PGDH activity and/or increase PGE2 activity and/or increase PGE2 synthesis and/or reduce 15-PGDH levels and/or increase PGE2 levels in the individual, thereby regenerating and/or rejuvenating the injured, damaged, aged, or diseased thyroid. In some cases, the administering is systemic administration (e.g., orally). In other cases, the administering is local administration (e.g., intravenously or subcutaneously). Any suitable administration route as described herein may be used, including intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

In various aspects, after administration of the 15-PGDH inhibitor and/or PGE2 activator, a function of the injured, damaged, aged, or diseased thyroid is regenerated or rejuvenated relative to a function of the injured, damaged, aged, or diseased thyroid prior to treatment with the 15-PGDH inhibitor and/or PGE2 activator. In various aspects, after administration of the 15-PGDH inhibitor and/or PGE2 activator, a function of the injured, damaged, aged, or diseased thyroid is regenerated or rejuvenated to a level substantially similar to a level found in young thyroid.

In some embodiments, after administration of the 15-PGDH inhibitor and/or PGE2 activator, the injured, damaged, aged, or diseased thyroid exhibits increased or enhanced thyroid tissue functions (e.g., as compared to the injured, damaged, aged, or diseased thyroid prior to treatment with the 15-PGDH inhibitor and/or PGE2 activator) as determined by: blood tests for measuring thyroid-stimulating hormone (TSH), triiodothyronine (T3), thyroxine (T4), or calcitonin; antibody tests for detecting thyroid hormones; or radioactive iodine uptake.

In some embodiments, after administration of the 15-PGDH inhibitor and/or PGE2 activator, the injured, damaged, aged, or diseased thyroid is capable of functioning at substantially similar to levels found in young thyroid. In some embodiments, after administration of the 15-PGDH inhibitor and/or PGE2 activator, the injured, damaged, aged, or diseased thyroid is capable of functioning such as regulating, producing, and secreting hormones at levels that are substantially similar to levels of function found in young thyroid.

In some embodiments, after administration of the 15-PGDH inhibitor and/or PGE2 activator, the injured, damaged, aged, or diseased thyroid exhibits decreased level of thyroid disease such as hyperthyroidism, hypothyroidism, Hashimoto's thyroiditis, Graves' disease, goiter, thyroid nodule, or thyroid cancer.

Regenerating and/or Rejuvenating Injured, Damaged, Aged, or Diseased Lung

Lung is an organ of the respiratory system and comprises various cell types in lung connective tissue, respiratory epithelium, bronchial airways, respiratory zone, or alveoli. Example cell types of lung include alveolar epitheliums. The lung's main function includes gas exchange between lung and blood; protection against respiratory pathogen or infection; maintaining homeostasis of pressure or acid-base in blood; or speech by providing air and airflow for the creation of vocal sound. During the aging process, one or more of these functions may be abrogated or reduced. In elderly people, lung function has been shown to be decreased or impaired.

Accordingly, in one aspect of the disclosure, a method is provided for regenerating and/or rejuvenating injured, damaged, aged, or diseased lung by administering to an individual (e.g., an aged individual, e.g., having an injured, damaged, aged, or diseased lung) or to an injured, damaged, aged, or diseased lung a 15-PGDH inhibitor and/or PGE2 activator in an amount effective to inhibit 15-PGDH activity and/or increase PGE2 activity and/or increase PGE2 synthesis and/or reduce 15-PGDH levels and/or PGE2 levels in the individual, thereby regenerating and/or rejuvenating the injured, damaged, aged, or diseased lung. In some cases, the administering is systemic administration (e.g., orally). In other cases, the administering is local administration (e.g., by inhalation or intranasally). Any suitable administration route as described herein may be used, including intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

In various aspects, after administration of the 15-PGDH inhibitor and/or PGE2 activator, a function of the injured, damaged, aged, or diseased lung is regenerated or rejuvenated relative to a function of the injured, damaged, aged, or diseased lung prior to treatment with the 15-PGDH inhibitor and/or PGE2 activator. In various aspects, after administration of the 15-PGDH inhibitor and/or PGE2 activator, a function of the injured, damaged, aged, or diseased lung is regenerated or rejuvenated to a level substantially similar to a level found in young lung.

In some embodiments, after administration of the 15-PGDH inhibitor and/or PGE2 activator, the injured, damaged, aged, or diseased lung exhibits increased or enhanced lung tissue functions (e.g., as compared to the injured, damaged, aged, or diseased lung prior to treatment with the 15-PGDH inhibitor and/or PGE2 activator) as determined by: evaluating lung capacity for volume or air inhaled or exhaled; pulmonary plethysmographs: spirometry: lung diffusing capacity: pulse oximetry: lung imaging; bronchoscopy; or thoracotomy.

In some embodiments, after administration of the 15-PGDH inhibitor and/or PGE2 activator, the injured, damaged, aged, or diseased lung is capable of functioning at substantially similar to levels found in young lung. In some embodiments, after administration of the 15-PGDH inhibitor and/or PGE2 activator, the injured, damaged, aged, or diseased lung exhibits decreased level of lung disease such as inflammation, infection, blood-supply change, obstructive lung disease, restrictive lung disease, congenital disorder, pneumothorax, lung nodule, or lung cancer. In some embodiments, after administration of the 15-PGDH inhibitor and/or PGE2 activator, the injured, damaged, aged, or diseased lung exhibits decreased level of lung fibrosis.

Regenerating and/or Rejuvenating Injured, Damaged, Aged, or Diseased Smooth Muscle

Smooth muscle is an involuntary non-striated muscle. It is divided into two subgroups: single-unit (unitary) and multiunit smooth muscle. Within single-unit cells, the whole bundle or sheet contracts as a syncytium. Smooth muscle cells (myocytes) are found in the walls of hollow organs, including the stomach, intestines, urinary bladder and uterus, and in the walls of passageways, such as the arteries and veins of the circulatory system, and the tracts of the respiratory, urinary, and reproductive systems. Smooth muscles' main function includes contraction and relaxation, which lead to movement of the digestive tract, movement of the autonomous nervous system (e.g., for breathing), or regulating homeostasis such as raising skin hair follicles for regulating body temperature. During the aging process, one or more of these functions may be abrogated or reduced. In elderly people, smooth muscle function has been shown to be decreased or impaired.

Accordingly, in one aspect of the disclosure, a method is provided for regenerating and/or rejuvenating injured, damaged, aged, or diseased smooth muscle by administering to an individual (e.g., an aged individual, e.g., having an injured, damaged, aged, or diseased smooth muscle) or to an injured, damaged, aged, or diseased smooth muscle a 15-PGDH inhibitor and/or PGE2 activator in an amount effective to inhibit 15-PGDH activity and/or increase PGE2 activity and/or increase PGE2 synthesis and/or reduce 15-PGDH levels and/or increase PGE2 levels in the individual, thereby regenerating and/or rejuvenating the injured, damaged, aged, or diseased smooth muscle. In some cases, the administering is systemic administration (e.g., orally). In other cases, the administering is local administration (e.g., by intramuscularly). Any suitable administration route as described herein may be used, including intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

In various aspects, after administration of the 15-PGDH inhibitor and/or PGE2 activator, a function of the injured, damaged, aged, or diseased smooth muscle is regenerated or rejuvenated relative to a function of the injured, damaged, aged, or diseased smooth muscle prior to treatment with the 15-PGDH inhibitor and/or PGE2 activator. In various aspects, after administration of the 15-PGDH inhibitor and/or PGE2 activator, a function of the injured, damaged, aged, or diseased smooth muscle is regenerated or rejuvenated to a level substantially similar to a level found in young smooth muscle.

In some embodiments, after administration of the 15-PGDH inhibitor and/or PGE2 activator, the injured, damaged, aged, or diseased smooth muscle exhibits increased or enhanced smooth muscle functions (e.g., as compared to the injured, damaged, aged, or diseased smooth muscle prior to treatment with the 15-PGDH inhibitor and/or PGE2 activator) as determined by: blood test for measuring antibody associated with smooth muscle or medical imaging.

In some embodiments, after administration of the 15-PGDH inhibitor and/or PGE2 activator, the injured, damaged, aged, or diseased smooth muscle is capable of functioning at substantially similar to levels found in young smooth muscle. In some embodiments, after administration of the 15-PGDH inhibitor and/or PGE2 activator, the injured, damaged, aged, or diseased smooth muscle exhibits decreased level of smooth muscle disease such as multisystemic smooth muscle dysfunction syndrome, blood vessel disorders, atherosclerosis, or inflammation. In some embodiments, after administration of the 15-PGDH inhibitor and/or PGE2 activator, the injured, damaged, aged, or diseased smooth muscle exhibits decreased level of smooth muscle fibrosis.

Regenerating and/or Rejuvenating Injured, Damaged, Aged, or Diseased Blood

Blood is a body fluid that delivers necessary substances such as nutrients and oxygen to the cells and transports metabolic waste products away from those same cells and comprises blood plasma (about 55% of blood fluid,) comprising proteins, glucose, mineral ions, hormones, carbon dioxide (plasma being the main medium for excretory product transportation), and blood cells, mainly red blood cells (RBCs or erythrocytes), white blood cells (WBCs or leukocytes), and platelets. Blood is circulated around the body through blood vessels by the pumping action of the heart. Arterial blood carries oxygen from inhaled air to the tissues of the body, and venous blood carries carbon dioxide, a waste product of metabolism produced by cells, from the tissues to the lungs to be exhaled. Blood's main function includes supply of oxygen to tissues (bound to hemoglobin in red blood cells); supply of nutrients such as glucose, amino acids, or fatty acids (dissolved in the blood or bound to plasma proteins): removal of waste such as carbon dioxide, urea, or lactic acid: immune response, including circulation of white blood cells and detection of foreign material by antibodies: coagulation, messenger function, including transport of hormones: or regulation of core body temperature. During the aging process, one or more of these functions may be abrogated or reduced. In elderly people, blood function has been shown to be decreased or impaired.

Accordingly, in one aspect of the disclosure, a method is provided for regenerating and/or rejuvenating injured, damaged, aged, or diseased blood by administering to an individual (e.g., an aged individual, e.g., having an injured, damaged, aged, or diseased blood) or to an injured, damaged, aged, or diseased blood a 15-PGDH inhibitor and/or PGE2 activator in an amount effective to inhibit 15-PGDH activity and/or increase PGE2 activity and/or increase PGE2 synthesis and/or reduce 15-PGDH levels and/or increase PGE2 levels in the individual, thereby regenerating and/or rejuvenating the injured, damaged, aged, or diseased blood. In some cases, the administering is systemic administration (e.g., orally). In other cases, the administering is local administration (e.g., by intravenously or intraventricularly). Any suitable administration route as described herein may be used, including intrathecally, intraocularly, intravitreally, retinally, intravenously, intramuscularly, intraventricularly, intracerebrally, intracerebellarly, intracerebroventricularly, intraperenchymally, subcutaneously, or a combination thereof.

In various aspects, after administration of the 15-PGDH inhibitor and/or PGE2 activator, a function of the injured, damaged, aged, or diseased blood is regenerated or rejuvenated relative to a function of the injured, damaged, aged, or diseased blood prior to treatment with the 15-PGDH inhibitor and/or PGE2 activator. In various aspects, after administration of the 15-PGDH inhibitor and/or PGE2 activator, a function of the injured, damaged, aged, or diseased blood is regenerated or rejuvenated to a level substantially similar to a level found in young blood. In some embodiments, after administration of the 15-PGDH inhibitor and/or PGE2 activator, the injured, damaged, aged, or diseased blood exhibits restored or regenerated or rejuvenated serum cytokine levels that are substantially similar to serum cytokine levels found in a young individual. The restored or regenerated or rejuvenated serum cytokine levels in the treated injured, damaged, aged, or diseased blood can be any one or any combination of: interleukin-10 (IL10), interleukin-6 (IL6), betacellulin (BTC), granulocyte-macrophage colony-stimulating factor (GM-CSF), interleukin-13 (IL13), tumor necrosis factor alpha (TNF-α), interleukin-1 beta (IL1b), interleukin-22 (1L22).

In some embodiments, after administration of the 15-PGDH inhibitor and/or PGE2 activator, the injured, damaged, aged, or diseased blood exhibits increased or enhanced blood functions (e.g., as compared to the injured, damaged, aged, or diseased blood prior to treatment with the 15-PGDH inhibitor and/or PGE2 activator) as determined by: complete blood count; metabolic panel for measuring metabolites such as electrolytes, calcium, glucose, sodium, potassium, carbon dioxide, chloride, blood urea nitrogen (BUN), creatinine, albumin, total protein, alkaline phosphatase, alkaline aminotransferase, aspartate aminotransferase, or bilirubin; lipid panel; thyroid panel; enzyme markers; coagulation panel; dehydroepiandrosterone (DHEA)-sulfate serum test; C-reactive protein test; or circulating cytokines (e.g., by detecting and measuring serum cytokines are selected from the group consisting of: interleukin-10 (IL10), interleukin-6 (IL6), betacellulin (BTC), granulocyte-macrophage colony-stimulating factor (GM-CSF), interleukin-13 (IL13), tumor necrosis factor alpha (TNF-α), interleukin-1 beta (IL1b), interleukin-22 (IL22), or any combination thereof.)

In some embodiments, after administration of the 15-PGDH inhibitor and/or PGE2 activator, the injured, damaged, aged, or diseased blood is capable of functioning at substantially similar to levels found in young blood. In some embodiments, after administration of the 15-PGDH inhibitor and/or PGE2 activator, the injured, damaged, aged, or diseased blood is capable of functioning such as supply of oxygen to tissues (bound to hemoglobin in red blood cells): supply of nutrients such as glucose, amino acids, or fatty acids (dissolved in the blood or bound to plasma proteins); removal of waste such as carbon dioxide, urea, or lactic acid: immune response, including circulation of white blood cells and detection of foreign material by antibodies; coagulation, messenger function, including transport of hormones; or regulation of core body temperature at levels that are substantially similar to levels of function found in young blood. In some embodiments, after administration of the 15-PGDH inhibitor and/or PGE2 activator, the injured, damaged, aged, or diseased blood exhibits decreased level of blood disease such as anemia, hemophilia, blood clots, and blood cancers such as leukemia, lymphoma, or myeloma.

### Subjects

The subject can be any subject, e.g., a human or other mammal, with an age-related condition or at risk of having an age-related condition. In some embodiments, the subject is a human. In some embodiments, the subject is an adult. In some embodiments, the subject is a child (e.g., a child with a genetic disorder that causes premature aging, e.g., progeria). In some embodiments, the subject is female (e.g., an adult female). In some embodiments, the subject is male (e.g., an adult male).

In some embodiments, the subject is human, and the method further comprises a step in which the human is selected for treatment with the 15-PGDH inhibitor and/or PGE2 activator based on a diagnosis of an age-related condition or disease, or on the potential for or risk of developing an age-related condition or disease, or based on the age of the individual, based on the presence of one or more biomarkers of aging as described herein, or based on the potential for, the risk of developing, or a diagnosis of injury, damage, and/or disease to a tissue or organ. In some such embodiments, the human is selected based on his or her age. For example, a human can be selected for treatment based on age who is over 20, 25, 30, 35, 40, 45, 50, 55, 60, 65, 70, 75, 80, 85, 90, 95, or 100 years old or older, or any age in which the human has or potentially has an age-related condition or disease or has one or more biomarkers of aging as described herein. In some embodiments, the human is selected based on a potential for an age-related condition or disease, based on the presence or potential presence of an environmental, lifestyle, or medical factor linked to premature aging of one or more (e.g., skeletal muscle and/or non-skeletal muscle tissues and/or organs, such as smoking, drinking, diet, lack of physical activity, insufficient sleep, drug use, exposure to UV rays, exposure to extreme temperatures, stress, excess weight, or health-related factors such as infections, mental illness, cancer, diabetes, etc. In some embodiments, the subject has an age-related condition caused by premature aging of one or more tissues, e.g., a genetic disorder such as Osteogenesis imperfecta, Bloom syndrome, Cockayne Syndrome, Hutchinson-Gilford Progeria Syndrome, Mandibuloacral Dysplasia, Progeria, Progeroid Syndrome, Rothmund-Thomson Syndrome, Seip Syndrome, Werner Syndrome, Down Syndrome, Acrogeria, Rothmund-Thomson syndrome, an immunodeficiency of these tissues that lead to premature aging syndromes, such as Ataxia telangiectasia, or an infectious disease of these tissues that lead to premature aging syndromes, such as human immunodeficiency virus (HIV).

In some embodiments, the subject is determined to have injured, damaged, aged, or diseased tissues and/or organs or have an age-related condition or disease as determined using any method of assessing any measure of the function, performance, health, strength, endurance, physiological activity, or any other property of a (e.g., skeletal muscle and/or non-skeletal muscle) tissue and/or organ, e.g., a performance-based, imaging-based, physiological, molecular, cellular, or functional assay. For example, a heart can be assessed using any method of assessing heart function or health, such as angiograms, electrocardiograms, treadmill test, echocardiogram, etc. In some embodiments, the subject is selected for treatment based on a detection of elevated levels of 15-PGDH transcript or prostaglandin-E synthases transcript, protein, or enzymatic activity in a (e.g., skeletal muscle and/or non-skeletal muscle) related tissue and/or organ, or on a detection of decreased levels of PGE2 and/or PGD2 in the tissue and/or organ.

In some embodiments, the methods comprise an additional step subsequent to the administration of a 15-PGDH inhibitor and/or PGE2 activator, comprising assessing the health, function, performance, or any other property of a (e.g., skeletal muscle and/or non-skeletal muscle) tissue and/or organ in the subject, or comprising assessing the level of 15-PGDH (e.g., of 15-PGDH protein, transcript, or activity) and/or PGE2 and/or PGD2 in the (e.g., skeletal muscle and/or non-skeletal) muscle tissue and/or organ in the subject, e.g., to ascertain the potential effects of the prior administration of the 15-PGDH inhibitor and/or PGE2 activator on the tissue and/or organ. In some such embodiments, the health, function, performance, 15-PGDH level, 15-PGDH activity, PGE2 level. PGD2 level, or other property of the tissue and/or organ is detected or examined and compared to the health, function, performance, 15-PGDH level, 15-PGDH activity, PGE2 level, PGD2 level, or other property of the tissue and/or organ prior to the administration of the 15-PGDH inhibitor and/or PGE2 activator or to a control value, wherein a determination that the health, function, or performance of the tissue and/or organ has improved, that the 15-PGDH level has decreased, that the 15-PGDH activity has decreased, that the PGE2 level and/or PGD2 level has increased, in the tissue subsequent to the administration of the 15-PGDH inhibitor and/or PGE2 activator as compared to the value obtained prior to the administration of the 15-PGDH inhibitor and/or PGE2 activator or relative to a control value, indicates that the 15-PGDH inhibitor and/or PGE2 activator has had a beneficial effect in the (e.g., skeletal muscle and/or non-skeletal muscle) tissue and/or organ of the subject.

In some embodiments, the subject is suspected of having or at risk for developing the condition or disease associated with muscle damage, injury, or atrophy. In some cases, the condition or disease associated with muscle damage, injury or atrophy is selected from the group consisting of acute muscle injury or trauma, soft, tissue hand injury, spinal muscular atrophy (SMA). Duchenne muscular dystrophy (DMD), Becker muscular dystrophy, limb girdle muscular dystrophy, amyotrophic lateral sclerosis (ALS), distal muscular dystrophy (DD), inherited myopathies, myotonic muscular dystrophy (MOD), mitochondrial myopathies, myotubular myopathy (MM), myasthenia gravis (MG), congestive heart failure, periodic paralysis, polymyositis, rhabdomyolysis, dermatomyositis, cancer cachexia, AIDS cachexia, cardiac cachexia, stress induced urinary incontinence, and sarcopenia. In some embodiments, the subject has an age-related condition, disorder, or disease such as a cardiovascular disease or condition (e.g., atrial fibrillation, stroke, ischemic heart diseases, cardiomyopathies, endocarditis, intracerebral haemorrhage, hypertension), a chronic respiratory disease or condition (e.g., chronic obstructive pulmonary disease, asbestosis, silicosis), a nutritional disease or condition (e.g., trachoma, diarrheal diseases, encephalitis), a kidney disease or condition (e.g., chronic kidney diseases), a gastrointestinal or digestive disease or condition (e.g., NASH, pancreatitis, ulcer, intestinal obstruction), a neurological disorder (e.g., Alzheimer's, dementia, Parkinson's, cognitive decline), a sensory disorder (e.g., hearing loss, vision loss, loss of sense of smell or sense of taste, macular degeneration, retinitis pigmentosa, glaucoma), a skin or subcutaneous disease or condition (e.g., cellulitis, ulcer, fungal skin diseases, pyoderma), osteoporosis, osteoarthritis, rheumatoid arthritis, a genetic disease causing premature aging in one or more (e.g., skeletal muscle and/or non-skeletal muscle) tissues (e.g., progeria, osteogenesis imperfecta, Bloom syndrome, Cockayne Syndrome. Hutchinson-Gilford Progeria Syndrome, Mandibuloacral Dysplasia, Progeroid Syndrome, Rothmund-Thomson Syndrome, Seip Syndrome, Werner Syndrome, Down Syndrome, Acrogeria, Rothmund-Thomson syndrome), an immunodeficiency of these tissues that lead to premature aging syndromes (e.g., Ataxia telangiectasia), or an infectious disease of these tissues that leads to premature aging syndromes, (e.g., human immunodeficiency virus (HIV)), and the like.

The administration of the 15-PGDH inhibitor and/or PGE2 activator can provide improvement in any of these conditions, and can help improve, e.g., osteoporosis, hair loss, aged skin, cognitive disorders, sensory disorders, aged hematopoietic stem cell function, and gastrointestinal function, as described herein.

The present methods and compositions can be used to treat any (e.g., skeletal muscle and/or non-skeletal muscle) tissue, or organs including such tissues, or cells within such tissues, including epithelial tissue, nerve tissue, connective tissue, smooth muscle, cardiac muscle, epidermal tissues, vascular tissues, heart, kidney, brain, bone, cartilage, brown fat, spleen, liver, colon, sensory organs, thyroid, lung, blood, small intestine, dental tissue, ovaries or other reproductive tissue, hair, cochlea, oligodendrocytes, etc.

In some embodiments, subjects are identified for treatment based on a diagnosis of an age-related condition, disorder, or disease; based on a determination of the presence of or potential for age-related loss of (e.g., skeletal muscle and/or non-skeletal muscle) tissue and/or organ function, health, or performance; based on a subject's age, e.g., an age associated with an age-related condition or disease; or based on a detection of any of the herein-described features of injured, damaged, aged, or diseased (e.g., skeletal muscle and/or non-skeletal muscle) tissues and/or organs, e.g., of elevated levels of PGE2 and/or PGD2 metabolites such as 15-keto-PGE2. PGEM, 15-keto-PGD2, or 13,14-Dihydro-15-PGD2, of decreased levels of PGE2 and/or PGD2, of decreased protein synthesis, of decreased mitochondrial activity, of decreased signaling through the EP1, EP2, EP3, EP4, DP1, and/or DP2 receptors, of elevated expression of genes associated with the senescence phenotype such as p16 (Ink4a) or p21 (Cdkn1a), of shortened telomere length in cells of the tissue, of elevated numbers of senescent cells in a (e.g., skeletal muscle and/or non-skeletal muscle) tissue, or of elevated levels or activity of 15-PGDH, in particular in senescent cells, e.g., macrophages, fibroadipocytes, fibroblasts, endothelial cells, etc.

In some embodiments, the subject is a pet or a farm animal such as a porcine, bovine, ovine, poultry, or fish, and the methods are used, e.g., to enhance (e.g., skeletal muscle and/or non-skeletal muscle) tissue and/or organ function or health in an aging animal or in an animal having injured, damaged, or diseased tissues and/or organs. In some embodiments, a vector or expression cassette comprising a nucleic acid inhibitor of 15-PGDH or enhancer of PGE2 synthesis (e.g., prostaglandin-E synthases), e.g., an shRNA, is introduced into the animal such that the nucleic acid inhibitor or enhancer is expressed in the cells of the animal, e.g., the cells of the (e.g., skeletal muscle and/or non-skeletal muscle) tissue and/or organ. In some embodiments, a vector or expression cassette comprising a polynucleotide encoding a polypeptide inhibitor of 15-PGDH or enhancer of PGE2 synthesis (e.g., prostaglandin-E synthases), e.g., an antibody or peptide, is introduced into the animal such that the polypeptide inhibitor or enhancer is expressed in the cells of the animal, e.g., the cells of the (e.g., skeletal muscle and/or non-skeletal muscle) tissue and/or organ. In some embodiments, gene therapy is used, e.g., such that all or part of an endogenous 15-PGDH encoding gene is replaced with a form of the gene that is less active, less stable, or less highly expressed in cells, e.g., skeletal muscle and/or non-skeletal muscle tissue and/or organ cells, of the animal. In some embodiments, gene therapy is used, e.g., such that all or part of an endogenous PGE2 synthase gene is replaced with a form of the gene that is more active, more stable, or more highly expressed in cells, e.g., skeletal muscle and/or non-skeletal muscle tissue and/or organ cells, of the animal. In some embodiments, modified RNA, e.g., a chemically modified RNA inhibitor such as shRNA or a chemically modified mRNA encoding a polypeptide 15-PGDH inhibitor and/or PGE2 activator is introduced into the animal such that the RNA inhibitor or activator, or the expressed inhibitor or activator, is present in cells of the animal.

### 5. Assessing 15-PGDH and/or PGE2 Levels

Any of a number of methods can be used to assess the level of 15-PGDH and/or PGE2 in a (e.g., skeletal muscle and/or non-skeletal muscle) tissue and/or organ, e.g., when using 15-PGDH or PGE2 as a biomarker or when assessing the efficacy of an inhibitor of 15-PGDH or an activator of PGE2. For example, the level of 15-PGDH and PGE2 can be assessed by examining the transcription of a gene encoding 15-PGDH (e.g., the Hpgd gene) or enzyme involved in the synthesis of PGE2 (e.g., prostaglandin-E synthases), by examining the levels of 15-PGDH protein or PGE2 in the tissue and/or organ (e.g., skeletal muscle and/or non-skeletal muscle tissue and/or organ), or by measuring the 15-PGDH enzyme activity or PGE2 activity in the tissue and/or organ (e.g., skeletal muscle and/or non-skeletal muscle tissue and/or organ). Such methods can be performed on the overall tissue and/or organ or on a subset of cells within the tissue and/or organ, e.g., senescent cells.

In some embodiments, the methods involve the measurement of 15-PGDH enzyme activity. e.g., using standard methods such as incubating a candidate compound in the presence of 15-PGDH enzyme, NAD(+), and PGE2 in an appropriate reaction buffer, and monitoring the generation of NADH (see, e.g., Zhang et al., (2015) Science 348: 1224), or by using any of a number of available kits such as the fluorometric PicoProbe 15-PGDH Activity Assay Kit (BioVision), or by using any of the methods and/or indices described in, e.g., publication EP2838533.

In some embodiments, the methods involve the detection of 15-PGDH-encoding or PGE2 enzyme (e.g., prostaglandin-E synthases) polynucleotide (e.g., mRNA) expression, which can be analyzed using routine techniques such as RT-PCR, Real-Time RT-PCR, semi-quantitative RT-PCR, quantitative polymerase chain reaction (qPCR), quantitative RT-PCR (qRT-PCR), multiplexed branched DNA (bDNA) assay, microarray hybridization, or sequence analysis (e.g., RNA sequencing (“RNA-Seq”)). Methods of quantifying polynucleotide expression are described, e.g., in Fassbinder-Orth, Integrative and Comparative Biology, 2014, 54:396-406; Thellin et al., Biotechnology Advances, 2009, 27:323-333; and Zheng et al., Clinical Chemistry, 2006, 52:7 (doi: 10/1373/clinchem.2005.065078). In some embodiments, real-time or quantitative PCR or RT-PCR is used to measure the level of a polynucleotide (e.g., mRNA) in a biological sample. See, e.g., Nolan et al., Nat. Protoc, 2006, 1:1559-1582. Wong et al., BioTechniques, 2005, 39:75-75. Quantitative PCR and RT-PCR assays for measuring gene expression are also commercially available (e.g., TaqMan® Gene Expression Assays, ThermoFisher Scientific).

In some embodiments, the methods involve the detection of 15-PGDH protein or PGE2 synthesis or stability, e.g., using routine techniques such as immunoassays, two-dimensional gel electrophoresis, and quantitative mass spectrometry that are known to those skilled in the art. Protein quantification techniques are generally described in “Strategies for Protein Quantitation,” Principles of Proteomics, 2nd Edition, R. Twyman, ed., Garland Science, 2013. In some embodiments, protein (e.g., 15-PGDH) or signaling molecule (e.g., PGE2) expression or stability is detected by immunoassay, such as but not limited to enzyme immunoassays (EIA) such as enzyme multiplied immunoassay technique (EMIT), enzyme-linked immunosorbent assay (ELISA). IgM antibody capture ELISA (MAC ELISA), and microparticle enzyme immunoassay (MEIA); capillary electrophoresis immunoassays (CEIA); radioimmunoassays (RIA); immunoradiometric assays (IRMA); immunofluorescence (IF); fluorescence polarization immunoassays (FPIA); and chemiluminescence assays (CL). If desired, such immunoassays can be automated. Immunoassays can also be used in conjunction with laser induced fluorescence (see, e.g., Schmalzing et al., Electrophoresis, 18:2184-93 (1997); Bao, J. Chromatogr. B. Biomed. Sci., 699:463-80 (1997)).

6. 15-PGDH and/or PGE2 as a Biomarker

In some embodiments, 15-PGDH and/or PGE2 may be used as a biomarker for injured, damaged, aged, or diseased (e.g., skeletal muscle and/or non-skeletal muscle) tissue and/or organs, or for the presence or potential for an age-related condition or disease. For example, a detection of an increase in 15-PGDH levels or decrease in PGE2 levels in a (e.g., skeletal muscle and/or non-skeletal muscle) tissue and/or organ, e.g., in the overall tissue and/or organ or in specific cells within the tissue and/or organ such as senescent cells, is indicative of aging in the tissue and/or organ, of a loss or decrease of function or health of the tissue and/or organ related to aging, or of the presence of an age-related condition or disease. For example, a detected increase of 15-PGDH or decrease in PGE2 of about 5%, about 10%, about 20%, about 30%, about 40%, about 50%, about 60%, about 70%, about 80%, about 90%, about 100%, or more 15-PGDH or PGE2, respectively in a (e.g., skeletal muscle and/or non-skeletal muscle) tissue and/or organ as compared to in a control tissue and/or organ from a subject without an age-related condition or disease may be indicative of aging of the tissue and/or organ, of a loss or decrease of function or health of the tissue and/or organ related to aging, or of the presence of an age-related condition or disease.

7. 15-PGDH Inhibitor and/or PGE2 Activators

Any agent that reduces, decreases, counteracts, attenuates, inhibits, blocks, downregulates, or eliminates in any way the expression, stability, or activity, e.g., enzymatic activity, of 15-PGDH can be used in the present methods. Inhibitors can be peptides, polypeptides, nucleic acids, antibodies, e.g., blocking antibodies or nanobodies, or any other molecule that reduces, decreases, counteracts, attenuates, inhibits, blocks, downregulates, or eliminates in any way the expression, stability, and/or activity of 15-PGDH, e.g., the enzymatic activity of 15-PGDH. Inhibitors may act at the level of nucleic acids, including reducing, decreasing, counteracting, attenuating, inhibiting, blocking, downregulating, or eliminating in any way the expression of 15-PGDH, for example, by inhibiting 15-PGDH promoter or enhancer activity, including, for example, reducing or decreasing the level of transcription factors associated with aging.

In addition, any agent that increases, activates, switches on, initiates, triggers or stimulates in any way the expression, stability, or activity, e.g., expression of PGE-2, can be used in the present methods. Activators can be peptides, polypeptides, nucleic acids, antibodies, nanobodies, or any other molecule that increases, activates, switches on, initiates, triggers or stimulates in any way the expression, stability, and/or activity of PGE-2, e.g., the expression of PGE-2. Activators or enhancers of PGE-2 expression may act at the level of nucleic acids or protein synthesis, including increasing, activating, switching on, initiating, triggering or stimulating in any way the expression of PGE-2, for example, by activating or enhancing PGE-2 promoter or enhancer activity, or increasing, activating, switching on, initiating, triggering or stimulating, for example COX1 and COX2 enzyme activity. Increasing, activating, switching on, initiating, triggering or stimulating expression of PGE-2 may also occur through inhibiting or decreasing activity of inhibitors of PGE-2 expression, including, for example, PGE-2 synthase levels.

In some embodiments, the 15-PGDH inhibitor and/or PGE2 activator decreases the activity, stability, or expression of 15-PGDH by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or more relative to a control level, e.g., in the absence of the inhibitor, in vivo or in vitro.

The efficacy of inhibitors can be assessed, e.g., by measuring 15-PGDH enzyme activity. e.g., using standard methods such as incubating a candidate compound in the presence of 15-PGDH enzyme, NAD(+), and PGE2 in an appropriate reaction buffer, and monitoring the generation of NADH (see, e.g., Zhang et al., (2015) Science 348: 1224), or by using any of a number of available kits such as the fluorometric PicoProbe 15-PGDH Activity Assay Kit (BioVision), or by using any of the methods and/or indices described in, e.g., publication EP2838533.

The efficacy of inhibitors can also be assessed, e.g., by detection of decreased polynucleotide (e.g., mRNA) expression, which can be analyzed using routine techniques such as RT-PCR, Real-Time RT-PCR, semi-quantitative RT-PCR, quantitative polymerase chain reaction (qPCR), quantitative RT-PCR (qRT-PCR), multiplexed branched DNA (bDNA) assay, microarray hybridization, or sequence analysis (e.g., RNA sequencing (“RNA-Seq”)). Methods of quantifying polynucleotide expression are described, e.g., in Fassbinder-Orth, Integrative and Comparative Biology, 2014, 54:396-406; Thellin et al., Biotechnology Advances, 2009, 27:323-333; and Zheng et al., Clinical Chemistry, 2006, 52:7 (doi: 10/1373/clinchem.2005.065078). In some embodiments, real-time or quantitative PCR or RT-PCR is used to measure the level of a polynucleotide (e.g., mRNA) in a biological sample. See, e.g., Nolan et al., Nat. Protoc, 2006, 1:1559-1582; Wong et al., BioTechniques, 2005, 39:75-75. Quantitative PCR and RT-PCR assays for measuring gene expression are also commercially available (e.g., TaqMan® Gene Expression Assays, ThermoFisher Scientific).

In some embodiments, the 15-PGDH inhibitor and/or PGE2 activator is considered effective if the level of expression of a 15-PGDH-encoding polynucleotide is decreased by at least about 5%, at least about 10%, at least about 20%, at least about 25%, at least about 30%, at least about 40%, at least about 50%, at least about 60%, at least about 70%, at least about 80%, at least about 90% or more as compared to the reference value, e.g., the value in the absence of the inhibitor, in vitro or in vivo. In some embodiments, a 15-PGDH inhibitor and/or PGE2 activator is considered effective if the level of expression of a 15-PGDH-encoding polynucleotide is decreased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, or more as compared to the reference value.

The effectiveness of a 15-PGDH inhibitor and/or PGE2 activator can also be assessed by detecting protein expression or stability, e.g., using routine techniques such as immunoassays, two-dimensional gel electrophoresis, and quantitative mass spectrometry that are known to those skilled in the art. Protein quantification techniques are generally described in “Strategies for Protein Quantitation,” Principles of Proteomics, 2nd Edition, R. Twyman, ed., Garland Science, 2013. In some embodiments, protein expression or stability is detected by immunoassay, such as but not limited to enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); immunofluorescence (IF); fluorescence polarization immunoassays (FPIA); and chemiluminescence assays (CL). If desired, such immunoassays can be automated. Immunoassays can also be used in conjunction with laser induced fluorescence (see, e.g., Schmalzing et al., Electrophoresis, 18:2184-93 (1997): Bao, J. Chromatogr. B. Biomed Sci., 699:463-80 (1997)).

For determining whether 15-PGDH protein levels are decreased in the presence of a 15-PGDH inhibitor and/or PGE-2 protein levels are increased in the presence of a PGE2 activator, the method comprises comparing the level of the protein (e.g., 15-PGDH protein or PGE-2) in the presence of the 15-PGDH inhibitor and/or PGE2 activator to a reference value, e.g., the level in the absence of the 15-PGDH inhibitor and/or PGE2 activator. In some embodiments, a 15-PGDH protein is decreased in the presence of an inhibitor if the level of the 15-PGDH protein is decreased by at least about 5%, at least about 10%, at least about 20%, at least about 30%, at least about 40%, at least about 50%, at least about 60%, at least about 70%, at least about 80%, at least about 90%/6, or more as compared to the reference value. In some embodiments, a 15-PGDH protein is decreased in the presence of an inhibitor if the level of the 15-PGDH protein is decreased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, or more as compared to the reference value. In some embodiments, a PGE-2 protein is decreased in the presence of an inhibitor if the level of the PGE-2 protein is decreased by at least about 5%, at least about 10%, at least about 20%, at least about 30%, at least about 40%, at least about 50%, at least about 60%, at least about 70%, at least about 80%, at least about 90%, or more as compared to the reference value. In some embodiments, a PGE-2 protein is decreased in the presence of an inhibitor if the level of the PGE-2 protein is decreased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, or more as compared to the reference value.

### Small Molecules

In particular embodiments, 15-PGDH is inhibited by the co-administration of a small molecule inhibitor, for example, as a combination therapy. Any small molecule inhibitor can be used that reduces, e.g., by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or more, the expression, stability, or activity of 15-PGDH relative to a control, e.g., the expression, stability, or activity in the absence of the 15-PGDH inhibitor and/or PGE2 activator. In particular embodiments, small molecule inhibitors may be used that can reduce the enzymatic activity of 15-PGDH in vitro or in vivo. Non-limiting examples of small molecule compounds that can be used in the present methods include the small molecules disclosed in publication EP 2838533, the entire disclosure of which is herein incorporated by reference. Small molecules can include, inter alia, the small molecules disclosed in Table 2 of publication EP 2838533, i.e., SW033291, SW033291 isomer B, SW033291 isomer A, SW033292, 413423, 980653, 405320, SW208078, SW208079, SW033290, SW208080, SW208081, SW206976, SW206977, SW206978, SW206979, SW206980, SW206992, SW208064, SW208065, SW208066, SW208067, SW208068, SW208069, SW208070, as well as combinations, derivatives, isomers, or tautomers thereof. In particular embodiments, the 15-PGDH inhibitor used is SW033291 (2-(butylsulfinyl)-4-phenyl-6-(thiophen-2-yl)thieno[2,3-b]pyridin-3-amine; PubChem CID: 3337839).

In some embodiments, the 15-PGDH inhibitor is a thiazolidinedione derivative (e.g., benzylidenethiazolidine-2,4-dione derivative) such as (5-(4-(2-(thiophen-2-yl)ethoxy)benzylidene)thiazolidine-2,4-dione), 5-(3-chloro-4-phenylethoxybenzylidene)thiazolidine-2,4-dione, 5-(4-(2-cyclohexylethoxv)benzylidene)thiazolidine-2,4-dione, 5-(3-chloro-4-(2-cyclohexylethoxy)benzyl)thiazolidine-2,4-dione, (Z)—N-benzyl-4-((2,4-dioxothiazolidin-5-ylidene)methyl)benzamide, or any of the compounds disclosed in Choi et al. (2013) Bioorganic & Medicinal Chemistry 21:4477-4484; Wu et al. (2010) Bioorg. Med. Chem. 18(2010) 1428-1433; Wu et al. (2011) J. Med Chem. 54:5260-5264; or Yu et al. (2019) Biotechnology and Bioprocess Engineering 24:464-475, the entire disclosures of which are herein incorporated by reference. In some embodiments, the 15-PGDH inhibitor is a COX inhibitor or chemopreventive agent such as ciglitazone (CID: 2750), or any of the compounds disclosed in Cho et al. (2002) Prostaglandins, Leukotrienes and Essential Fatty Acids 67(6):461-465, the entire disclosure of which is herein incorporated by reference.

In some embodiments, the 15-PGDH inhibitor is a compound containing a benzimidazole group, such as (1-(4-methoxyphenyl)-1H-benzo[d]imidazol-5-yl)(piperidin-1-yl)methanone (CID: 3474778), or a compound containing a triazole group, such as 3-(2,5-dimethyl-1-(p-tolyl)-1H-pyrrol-3-yl)-6,7,8,9-tetrahydro-5H-[1,2,4]triazolo[4,3-a]azepine (CID: 71307851), or any of the compounds disclosed in Duveau et al. (2015) (“Discovery of two small molecule inhibitors, ML387 and ML388, of human NAD+-dependent 15-hydroxyprostaglandin dehydrogenase,” published in Probe Reports from the NIH Molecular Libraries Program [Internet]), the entire disclosure of which is herein incorporated by reference. In some embodiments, the 15-PGDH inhibitor is 1-(3-methylphenyl)-1H-benzimidazol-5-yl)(piperidin-1-yl)methanone (CID: 4249877) or any of the compounds disclosed in Niesen et al. (2010) PLoS ONE 5(11):e13719, the entire disclosure of which is herein incorporated by reference. In some embodiments, the 15-PGDH inhibitor and/or PGE2 activator is 2-((6-bromo-4H-imidazo[4,5-b]pyridin-2-ylthio)methyl)benzonitrile (CID: 3245059), piperidin-1-yl(1-m-tolyl-1H-benzo[d]imidazol-5-yl)methanone (CID: 3243760), or 3-(2,5-dimethyl-1-phenyl-1H-pyrrol-3-yl)-6,7,8,9-tetrahydro-5H-[1,2,4]triazolo[4,3-a]azepine (CID: 2331284), or any of the compounds disclosed in Jadhav et al. (2011) (“Potent and selective inhibitors of NAD+-dependent 15-hydroxyprostaglandin dehydrogenase (HPGD),” published in Probe Reports from the NIH Molecular Libraries Program [Internet]), the entire disclosure of which is herein incorporated by reference.

In some embodiments, the 15-PGDH inhibitor is TD88 or any of the compounds disclosed in Seo et al. (2015) Prostaglandins, Leukotrienes and Essential Fatty Acids 97:35-41, or Shao et al. (2015) Genes & Diseases 2(4):295-298, the entire disclosures of which are herein incorporated by reference. In some embodiments, the 15-PGDH inhibitor and/or PGE2 activator is EEAH (Ethanol extract of Artocarpus heterophyllus) or any of the compounds disclosed in Karna (2017) Pharmacogn Mag. 2017 January; 13(Suppl 1): S122-S126, the entire disclosure of which is herein incorporated by reference.

### PGE2 Activators

In some embodiments, the PGE2 activator is any one of small molecule compounds, peptides, polypeptides, nucleic acids, antibodies, nanobodies, or any other molecule that increases, activates, switches on, initiates, triggers or stimulates in any way the expression, stability, and/or activity of PGE2, e.g., the expression of PGE2. Activators or enhancers of PGE2 expression may act at the level of nucleic acids or protein synthesis, including increasing, activating, switching on, initiating, triggering or stimulating in any way the expression of PGE2, for example, by activating or enhancing PGE2 promoter or enhancer activity, or increasing, activating, switching on, initiating, triggering or stimulating, for example Cox1 and Cox2 enzyme activity. Increasing, activating, switching on, initiating, triggering or stimulating expression of PGE2 may also occur through inhibiting or decreasing activity of inhibitors of PGE2 expression, including, for example, PGE2 synthase levels.

### Inhibitory Nucleic Acids

In some embodiments, the agent comprises an inhibitory nucleic acid, e.g., antisense DNA or RNA, morpholino oligomer, small interfering RNA (siRNA), microRNA (miRNA), or short hairpin RNA (shRNA). In some embodiments, the inhibitory RNA targets a sequence that is identical or substantially identical (e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical) to a target sequence in a 15-PGDH polynucleotide (e.g., a portion comprising at least 20, at least 30, at least 40, at least 50, at least 60, at least 70, at least 80, at least 90, or at least 100 contiguous nucleotides, e.g., from 20-500, 20-250, 20-100, 50-500, or 50-250 contiguous nucleotides of a 15-PGDH-encoding polynucleotide sequence (e.g., the human HPGD gene, Gene ID: 3248, including of any of its transcript variants, e.g., as set forth in GenBank Accession Nos. NM_000860.6, NM_001145816.2, NM_001256301.1, NM_001256305.1. NM_001256306.1, NM_001256307.1, or NM_001363574.1).

In some embodiments, the methods described herein comprise treating a subject, e.g., a subject with an age-related condition, disorder, or disease, or with injured, damaged, aged, or diseased tissues and/or organs using an shRNA or siRNA. A shRNA is an artificial RNA molecule with a hairpin turn that can be used to silence target gene expression via the siRNA it produces in cells. See, e.g., Fire et. al., Nature 391:806-811, 1998: Elbashir et al., Nature 411:494-498, 2001; Chakraborty et al., Mol Ther Nucleic Acids 8:132-143, 2017; and Bouard et al., Br. J. Pharmacol. 157:153-165, 2009. In some embodiments, a method of treating a subject, e.g., a subject with an age-related condition, disorder, or disease, or a subject with injured, damaged, aged, or diseased tissues and/or organs comprises administering to the subject a therapeutically effective amount of a modified RNA or a vector comprising a polynucleotide that encodes an shRNA or siRNA capable of hybridizing to a portion of a 15-PGDH mRNA (e.g., a portion of the human 15-PGDH-encoding polynucleotide sequence set forth in any of GenBank Accession Nos. NM_000860.6. NM_001145816.2, NM_001256301.1, NM_001256305.1, NM_001256306.1, NM_001256307.1, or NM_001363574.1). In some embodiments, the vector further comprises appropriate expression control elements known in the art, including, e.g., promoters (e.g., inducible promoters or tissue specific promoters), enhancers, and transcription terminators.

In some embodiments, the agent is a 15-PGDH-specific microRNA (miRNA or miR). A microRNA is a small non-coding RNA molecule that functions in RNA silencing and post-transcriptional regulation of gene expression. miRNAs base pair with complementary sequences within the mRNA transcript. As a result, the mRNA transcript may be silenced by one or more of the mechanisms such as cleavage of the mRNA strand, destabilization of the mRNA through shortening of its poly(A) tail, and decrease in the translation efficiency of the mRNA transcript into proteins by ribosomes.

In some embodiments, the agent may be a morpholino oligomer, an antisense oligonucleotide, e.g., an RNase H-dependent antisense oligonucleotide (ASO), or combinations thereof. ASOs are single-stranded, chemically modified oligonucleotides that bind to complementary sequences in target mRNAs and reduce gene expression both by RNase H-mediated cleavage of the target RNA and by inhibition of translation by steric blockade of ribosomes. In some embodiments, the oligonucleotide is capable of hybridizing to a portion of a 15-PGDH mRNA (e.g., a portion of a human 15-PGDH-encoding polynucleotide sequence as set forth in any of GenBank Accession Nos. NM_000860.6, NM_001145816.2, NM_001256301.1, NM_001256305.1, NM_001256306.1, NM_001256307.1, or NM_001363574.1). In some embodiments, the oligonucleotide has a length of about 10-30 nucleotides (e.g., 10, 12, 14, 16, 18, 20, 22, 24, 26, 28, or 30 nucleotides). In some embodiments, the oligonucleotide has 100% complementarity to the portion of the mRNA transcript it binds. In other embodiments, the DNA oligonucleotide has less than 100% complementarity (e.g., about 95%, about 90%, about 85%, about 80%, about 75%, or about 70% complementarity) to the portion of the mRNA transcript it binds, but can still form a stable RNA:DNA duplex for the RNase H to cleave the mRNA transcript.

Suitable antisense molecules, siRNA, miRNA, and shRNA can be produced by standard methods of oligonucleotide synthesis or by ordering such molecules from a contract research organization or supplier by providing the polynucleotide sequence being targeted. The manufacture and deployment of such antisense molecules in general terms may be accomplished using standard techniques described in contemporary reference texts: for example, Gene and Cell Therapy: Therapeutic Mechanisms and Strategies. 4^{th} edition by N. S. Templeton; Translating Gene Therapy to the Clinic: Techniques and Approaches, 1^{st} edition by J. Laurence and M. Franklin; High-Throughput RNAi Screening: Methods and Protocols (Methods in Molecular Biology) by D. O. Azorsa and S. Arora; and Oligonucleotide-Based Drugs and Therapeutics: Preclincal and Clinical Considerations by N. Ferrari and R. Segui.

Inhibitory nucleic acids can also include morpholino oligomers (DNA bases attached to a backbone of methylenemorpholine rings linked through phosphordiamidate groups), which block access to short (approximately 25 bases) specific sequences of the base-pairing surfaces of ribonucleic acid and are used to knock down gene function. Also included are RNA aptamers, which are short, synthetic oligonucleotide sequences that bind to proteins (see, e.g., Li et al., Nuc. Acids Res. (2006), 34:6416-24). Both are notable for both high affinity and specificity for the targeted molecule, and have the additional advantage of being smaller than antibodies (usually less than 6 kD). RNA aptamers with a desired specificity are generally selected from a combinatorial library, and can be modified to reduce vulnerability to ribonucleases, using methods known in the art.

### Antibodies

In some embodiments, the agent is an anti-15-PGDH antibody or an antigen-binding fragment thereof. In some embodiments, the antibody is a blocking antibody (e.g., an antibody that binds to a target and directly interferes with the target's function, e.g., 15-PGDH enzyme activity). In some embodiments, the antibody is a neutralizing antibody (e.g., an antibody that binds to a target and negates the downstream cellular effects of the target). In some embodiments, the antibody binds to human 15-PGDH.

In some embodiments, the antibody is a monoclonal antibody. In some embodiments, the antibody is a polyclonal antibody. In some embodiments, the antibody is a chimeric antibody. In some embodiments, the antibody is a humanized antibody. In some embodiments, the antibody is a human antibody. In some embodiments, the antibody is an antigen-binding fragment, such as a F(ab′)2, Fab′, Fab, scFv, and the like. The term “antibody or antigen-binding fragment” can also encompass multi-specific and hybrid antibodies, with dual or multiple antigen or epitope specificities.

In some embodiments, an anti-15-PGDH antibody comprises a heavy chain sequence or a portion thereof, and/or a light chain sequence or a portion thereof, of an antibody sequence disclosed herein. In some embodiments, an anti-15-PGDH antibody comprises one or more complementarity determining regions (CDRs) of an anti-15-PGDH antibody as disclosed herein. In some embodiments, an anti-15-PGDH antibody is a nanobody, or single-domain antibody (sdAb), comprising a single monomeric variable antibody domain, e.g., a single VHH domain.

For preparing an antibody that binds to 15-PGDH, many techniques known in the art can be used. See, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2nd ed. 1986)). In some embodiments, antibodies are prepared by immunizing an animal or animals (such as mice, rabbits, or rats) with an antigen for the induction of an antibody response. In some embodiments, the antigen is administered in conjugation with an adjuvant (e.g., Freund's adjuvant). In some embodiments, after the initial immunization, one or more subsequent booster injections of the antigen can be administered to improve antibody production. Following immunization, antigen-specific B cells are harvested, e.g., from the spleen and/or lymphoid tissue. For generating monoclonal antibodies, the B cells are fused with myeloma cells, which are subsequently screened for antigen specificity.

The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Additionally, phage or yeast display technology can be used to identify antibodies and heteromeric Fab fragments that specifically bind to selected antigens (see, e.g., McCafferty et al., Nature 348:552-554 (1990); Marks et al., Biotechnology 10:779-783 (1992); Lou et al. m PEDS 23:311 (2010); and Chao et al., Nature Protocols, 1:755-768 (2006)). Alternatively, antibodies and antibody sequences may be isolated and/or identified using a yeast-based antibody presentation system, such as that disclosed in, e.g., Xu et al., Protein Eng Des Sel, 2013, 26:663-670; WO 2009/036379; WO 2010/105256; and WO 2012/009568. Random combinations of the heavy and light chain gene products generate a large pool of antibodies with different antigenic specificity (see, e.g., Kuby, Immunology (3rd ed. 1997)). Techniques forthe production of single chain antibodies or recombinant antibodies (U.S. Pat. Nos. 4,946,778, 4,816,567) can also be adapted to produce antibodies.

Antibodies can be produced using any number of expression systems, including prokaryotic and eukaryotic expression systems. In some embodiments, the expression system is a mammalian cell, such as a hybridoma, or a CHO cell. Many such systems are widely available from commercial suppliers. In embodiments in which an antibody comprises both a VH and VL region, the VH and VL regions may be expressed using a single vector, e.g., in a di-cistronic expression unit, or be under the control of different promoters. In other embodiments, the VH and VL region may be expressed using separate vectors.

In some embodiments, an anti-15-PGDH antibody comprises one or more CDR, heavy chain, and/or light chain sequences that are affinity matured. For chimeric antibodies, methods of making chimeric antibodies are known in the art. For example, chimeric antibodies can be made in which the antigen binding region (heavy chain variable region and light chain variable region) from one species, such as a mouse, is fused to the effector region (constant domain) of another species, such as a human. As another example, “class switched” chimeric antibodies can be made in which the effector region of an antibody is substituted with an effector region of a different immunoglobulin class or subclass.

In some embodiments, an anti-15-PGDH antibody comprises one or more CDR, heavy chain, and/or light chain sequences that are humanized. For humanized antibodies, methods of making humanized antibodies are known in the art. See, e.g., U.S. Pat. No. 8,095,890. Generally, a humanized antibody has one or more amino acid residues introduced into it from a source which is non-human. As an alternative to humanization, human antibodies can be generated. As a non-limiting example, transgenic animals (e.g., mice) can be produced that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immun., 7:33 (1993); and U.S. Pat. Nos. 5,591,669, 5,589,369, and 5,545,807.

In some embodiments, antibody fragments (such as a Fab, a Fab′, a F(ab′)2, a scFv, nanobody, or a diabody) are generated. Various techniques have been developed for the production of antibody fragments, such as proteolytic digestion of intact antibodies (see, e.g., Morimoto et al., J. Biochem. Biophys. Meth., 24:107-117 (1992); and Brennan et al., Science, 229:81 (1985)) and the use of recombinant host cells to produce the fragments. For example, antibody fragments can be isolated from antibody phage libraries. Alternatively, Fab′-SH fragments can be directly recovered from E. coli cells and chemically coupled to form F(ab′)2 fragments (see, e.g., Carter et al., BioTechnology, 10:163-167 (1992)). According to another approach, F(ab′)2 fragments can be isolated directly from recombinant host cell culture. Other techniques for the production of antibody fragments will be apparent to those skilled in the art.

Methods for measuring binding affinity and binding kinetics are known in the art. These methods include, but are not limited to, solid-phase binding assays (e.g., ELISA assay), immunoprecipitation, surface plasmon resonance (e.g., Biacore™ (GE Healthcare, Piscataway, NJ)), kinetic exclusion assays (e.g., KinExA®), flow cytometrv, fluorescence-activated cell sorting (FACS), BioLayer interferometry (e.g., Octet™ (FortdBio, Inc., Menlo Park, CA)), and western blot analysis.

### Peptides

In some embodiments, the agent is a peptide, e.g., a peptide that binds to and/or inhibits the enzymatic activity or stability of 15-PGDH. In some embodiments, the agent is a peptide aptamer. Peptide aptamers are artificial proteins that are selected or engineered to bind to specific target molecules. Typically, the peptides include one or more peptide loops of variable sequence displayed by the protein scaffold. Peptide aptamer selection can be made using different systems, including the yeast two-hybrid system. Peptide aptamers can also be selected from combinatorial peptide libraries constructed by phage display and other surface display technologies such as mRNA display, ribosome display, bacterial display and yeast display. See, e.g., Reverdatto et al., 2015, Curr. Top. Med. Chem. 15:1082-1101.

In some embodiments, the agent is an affimer. Affimers are small, highly stable proteins, typically having a molecular weight of about 12-14 kDa, that bind their target molecules with specificity and affinity similar to that of antibodies. Generally, an affimer displays two peptide loops and an N-terminal sequence that can be randomized to bind different target proteins with high affinity and specificity in a similar manner to monoclonal antibodies. Stabilization of the two peptide loops by the protein scaffold constrains the possible conformations that the peptides can take, which increases the binding affinity and specificity compared to libraries of free peptides. Affimers and methods of making affimers are described in the art. See, e.g., Tiede et al., eLijfe, 2017, 6:e24903. Affimers are also commercially available, e.g., from Avacta Life Sciences.

### Vectors and Modified RNA

In some embodiments, polynucleotides providing 15-PGDH inhibiting activity or PGE2 upregulation, e.g., a nucleic acid inhibitor such as an siRNA or shRNA, or a polynucleotide encoding a polypeptide that inhibits 15-PGDH, or a nucleic acid that increases expression of PGE2 (for example, a transcription or activator of transcription), are introduced into cells, e.g., muscle cells, non-skeletal muscle tissue and/or organ cells, using an appropriate vector. Examples of delivery vectors that may be used with the present disclosure are viral vectors, plasmids, exosomes, liposomes, bacterial vectors, or nanoparticles. In some embodiments, any of the herein-described 15-PGDH inhibitor and/or PGE2 activators, e.g., a nucleic acid inhibitor or a polynucleotide encoding a polypeptide inhibitor, are introduced into cells, e.g., muscle cells, non-skeletal muscle tissue and/or organ cells, using vectors such as viral vectors. Suitable viral vectors include but not limited to adeno-associated viruses (AAVs), adenoviruses, and lentiviruses. In some embodiments, a 15-PGDH inhibitor and/or PGE2 activator or PGE2 activator. e.g., a nucleic acid inhibitor or expression activator or a polynucleotide encoding a polypeptide inhibitor or expression activator, is provided in the form of an expression cassette, typically recombinantly produced, having a promoter operably linked to the polynucleotide sequence encoding the inhibitor or activator. In some cases, the promoter is a universal promoter that directs gene expression in all or most tissue types; in other cases, the promoter is one that directs gene expression specifically in cells of the tissue being targeted.

In some embodiments, the nucleic acid or protein inhibitors of 15-PGDH are introduced into a subject, e.g., into the (e.g., skeletal muscle and/or non-skeletal muscle) tissues and/or organs of a subject, using modified RNA. Various modifications of RNA are known in the art to enhance, e.g., the translation, potency and/or stability of RNA, e.g., shRNA or mRNA encoding a 15-PGDH polypeptide inhibitor, when introduced into cells of a subject. In particular embodiments, modified mRNA (mmRNA) is used, e.g., mmRNA encoding a polypeptide inhibitor of 15-PGDH. In other embodiments, modified RNA comprising an RNA inhibitor of 15-PGDH expression is used, e.g., siRNA, shRNA, or miRNA. Non-limiting examples of RNA modifications that can be used include anti-reverse-cap analogs (ARCA), polyA tails of, e.g., 100-250 nucleotides in length, replacement of AU-rich sequences in the 3′UTR with sequences from known stable mRNAs, and the inclusion of modified nucleosides and structures such as pseudouridine, e.g., N-methylpseudouridine, 2-thiouridine, 4′thioRNA, 5-methylcytidine, 6-methyladenosine, amide 3 linkages, thioate linkages, inosine, 2-deoxyribonucleotides, 5-Bromo-uridine and 2′-O-methylated nucleosides. A non-limiting list of chemical modifications that can be used can be found, e.g., in the online database crdd.osdd.net/servers/sirnamod/. RNAs can be introduced into cells in vivo using any known method, including, inter alia, physical disturbance, the generation of RNA endocytosis by cationic carriers, electroporation, gene guns, ultrasound, nanoparticles, conjugates, or high-pressure injection. Modified RNA can also be introduced by direct injection, e.g., in citrate-buffered saline. RNA can also be delivered using self-assembled lipoplexes or polyplexes that are spontaneously generated by charge-to-charge interactions between negatively charged RNA and cationic lipids or polymers, such as lipoplexes, polyplexes, polycations and dendrimers. Polymers such as poly-L-lysine, polyamidoamine, and polyethyleneimine, chitosan, and poly(β-amino esters) can also be used. See, e.g., Youn et al. (2015) Expert Opin Biol Ther, September 2; 15(9): 1337-1348: Kaczmarek et al. (2017) Genome Medicine 9:60; Gan et al. (2019) Nature comm. 10: 871: Chien et al. (2015) Cold Spring Harb Perspect Med. 2015; 5:a014035; the entire disclosures of each of which are herein incorporated by reference.

### Gene Editing

Provided herein are nucleic acid molecules to inhibit 15-PGDH and/or activate or upregulate PGE2 synthesis. The nucleic acid molecules may be used in a TALEN (transcription activator-like effector nuclease technology), zinc-finger nuclease or CRISPR-Cas system to target a nucleic acid sequence for genetic screening, targeted transcriptional regulation, targeted knock-in, and targeted genome editing, including base editing, epigenetic editing, and introducing double strand breaks (DSBs) for homologous recombination-mediated insertion of a nucleotide sequence. Genome editing can refer to the targeted modification of a DNA sequence, including but not limited to, adding, removing, replacing, or modifying existing DNA sequences, and inducing chromosomal rearrangements or modifying transcription regulation elements (e.g., methylation/demethylation of a promoter sequence of a gene) to alter gene expression. In some embodiments, inhibiting 15-PGDH expression comprises inserting a silencer sequence near a polynucleotide sequence encoding 15-PGDH. In yet other embodiments, upregulating PGE2 synthesis comprises inserting an enhancer sequence near a polynucleotide sequence encoding PGE2.

CRISPR-Cas system requires a guide system that can locate Cas protein to the target DNA site in the genome. In some instances, the guide system comprises a crispr RNA (crRNA) with a 17-20 nucleotide sequence that is complementary to a target DNA site and a trans-activating crRNA (tracrRNA) scaffold recognized by the Cas protein (e.g., Cas9). The 17-20 nucleotide sequence complementary to a target DNA site is referred to as a spacer while the 17-20 nucleotide target DNA sequence is referred to a protospacer. While crRNAs and tracrRNAs exist as two separate RNA molecules in nature, single guide RNA (sgRNA or gRNA) can be engineered to combine and fuse crRNA and tracrRNA elements into one single RNA molecule. Thus, in one embodiment, the CRISPR guide system comprises two or more RNAs, e.g., crRNA and tracrRNA. In another embodiment, the CRISPR guide system comprises a sgRNA comprising a spacer sequence for genomic targeting and a scaffold sequence for Cas protein binding. In some instances, the guide system naturally comprises a sgRNA. For example, Cas12a/Cpf1 utilizes a guide system lacking tracrRNA and comprising only a crRNA containing a spacer sequence and a scaffold for Cas12a/Cpf1 binding. While the spacer sequence can be varied depending on a target site in the genome, the scaffold sequence for Cas protein binding can be identical for all gRNAs.

CRISPR-Cas systems described herein can comprise different CRISPR enzymes. For example, the CRISPR-Cas system can comprise Cas9, Cas12a/Cpf1, Cas12b/C2c1, Cas12c/C2c3, Cas12d/CasY, Cas12e/CasX, Cas12g, Cas12h, or Cas12i. Non-limiting examples of Cas enzymes include, but are not limited to, Cas1, CasiB, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpf1, Cas12b/C2c1, Cas12c/C2c3, Cas12d/CasY, Cas12e/CasX, Cas12f/Cas14/C2c10, Cas12g, Cas12h, Cas12i, Cas12k/C2c5, Cas13a/C2c2, Cas13b, Cas13c, Cas13d, C2c4, C2c8, C2c9, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4. Csd1, Csd2, Cst1, Cst2, Csh1, Csh2, Csa1, Csa2, Csa3, Csa4, Csa5, GSU0054, Type II Cas effector proteins, Type V Cas effector proteins, Type VI Cas effector proteins, CARF, DinG, homologues thereof, or modified or engineered versions thereof such as dCas9 (endonuclease-dead Cas9) and nCas9 (Cas9 nickase that has inactive DNA cleavage domain). In some cases, the compositions, methods, devices, and systems, described herein, may use the Cas9 nuclease from Streptococcus pyogenes, of which amino acid sequences and structures are well known to those skilled in the art.

Binding of a Cas protein to its target DNA sequence (i.e. target dsDNA) requires the presence of a protospacer adjacent motif (PAM), i.e. a short sequence adjacent to the protospacer, on the non-targeted DNA strand, which limits the region in the genome that can be targeted by Cas proteins. Cas proteins from different bacterial species recognize different PAM sequences and make cuts 3-4 nucleotides upstream (e.g., Cas9) or 18-23 nucleotides downstream (e.g., Cas12a/Cpf1) of the PAM sequence. For example, Cas9 from Streptococcus pyogene recognizes the PAM sequence 5′-NGG-3′ (wherein “N” can be any nucleotide) and cleaves 3 nucleotide upstream of the PAM sequence. In another example, Cas9 from Staphylococcus aureus recognizes the PAM sequence 5′-NNGRRN-3′ (wherein “N” can be any nucleotide) and cleaves 4 nucleotide upstream of the PAM sequence. Although the PAM sequence is essential for Cas-mediated cleavage, in some instances, the gRNA sequence does not comprise a PAM sequence.

Provided herein are nucleic acid molecules comprising a target sequence comprising a spacer that is complementary to a sequence at a target site in the genome. A spacer, as described herein, can comprise about 10 to about 25 nucleotides in length. For example, a spacer sequence that is complementary to a target site sequence in the genome can be about 10, 11, 12, 13, 14, 15, 16, 17, 18, 19, 20, 22, 23, 24, 25, 26, 27, 27, 28, 30, or more nucleotides in length. A target site, as described herein, can comprise a sequence of about 20 nucleotides immediately upstream or 5′ of the first nucleotide of the PAM. Provided herein are guide system nucleic acid molecules, wherein at least 1, at least 2, or at least 3 consecutive nucleotides at the 5′ terminus of the guide system nucleic acid molecules are 100% identical to the 3′ terminus of the target sequence in a genome. Guide system nucleic acid molecules synthesized by the compositions, methods, devises, and systems, described herein, exhibit improved or enhanced pairing with a target sequence via complementary base pairing with the target sequence in a target gene compared to a guide system nucleic acid molecule comprising additional 5′ terminal G nucleotide that is not present in the protospacer sequence or complementary to the 3′ terminus of the target sequence.

Provided herein are guide system nucleic acid molecules exhibiting enhanced editing efficiency of a target sequence when guide system nucleic acid molecules are contacted with the target sequence in a complex with a CRISPR-Cas system (e.g., CRISPR-Cas9, CRISPR-Cas12a/Cpf1, etc.). For example, gRNA (guide RNA) provided herein can exhibit enhanced editing efficiency of a target sequence when in complex with a CRISPR-Cas system compared to a gRNA comprising additional one or more 5′ terminal G nucleotides, wherein the additional one or more 5′ terminal G nucleotides are not present in the protospacer sequence or not complementary to one or more nucleotides at the 3′ terminus of the target sequence in a genome. As used herein, the term “codon” generally refers to three consecutive nucleotides, which may or may not encode an amino acid. As used herein, the efficiency of editing” or “editing efficiency” can refer to the ability of a guide system nucleic acid molecule directed effector protein (e.g., CRISPR-Cas protein) to modify a target DNA sequence. Non-limiting examples of modification of a target sequence can include introducing a double stranded break, modifying a nucleobase, inducing chromosomal rearrangements, and modifying methylation/demethylation of a promoter sequence of a gene. A target sequence may be located in a gene or in a promoter region in a genome. An effector protein may be a gRNA directed nuclease, e.g., Cas protein such as Cas9 or any other Cas protein described herein. The editing efficiency can be measured by using any methods well known to one skilled in the art. For example, the efficiency of genome editing or editing efficiency can be measured by using tracking of indels by decomposition (TIDE) analysis, surveyor nuclease assay, junction PCR, droplet digital PCR (ddPCR), denaturing high-performance liquid chromatography (DHPLC), PCR single-stranded conformational polymorphism (SSCP), high-resolution melting (HRM), restriction enzyme digestion-suppressed PCR (RE-PCR), engineered nuclease-induced translocations (ENIT), restriction enzyme digestion, Sanger DNA sequencing, deep sequencing such as next generation sequencing (NGS), or any combination thereof. The term “indel(s)”, as used herein, refers to the insertion or deletion of a nucleotide base within a nucleic acid. In some embodiments, the efficiency of genome editing, e.g., generating a double-strand break, can be measured by TIDE analysis, a three-step method whereby the region targeted by the nuclease (e.g., Cas9) is PCR-amplified from DNA isolated from cells transfected with CRISPR-Cas system and gRNAs. Amplicons of 500-1500 bp generated around the target site are subject to conventional Sanger DNA sequencing followed by analysis using the web-based TIDE software. Any sequence modifications made by the nuclease are visualized in a graph of the sequence with the aberrant base signal. The software also provides precise localization of break sites and estimated statistical significance of each indel. In some embodiments, the editing efficiency e.g., the efficiency of generating a DNA break in the intended target site, is at least 25%, 30%, 35%, 40%, 45%, 50%, 55%, 60%, 65%, 70%, 75%, 80%, 85%, 90%, 95%, or 99%.

Further provided herein are guide system nucleic acid molecules that exhibit reduced off-target editing (e.g., editing of a non-target sequence) when guide system nucleic acid molecules are contacted with the target sequence in a complex with a CRISPR-Cas system (e.g., CRISPR-Cas9, CRISPR-Cas12a/Cpf1, etc.). For example, gRNA provided herein can exhibit reduced off-target editing when in complex with a CRISPR-Cas system compared to a gRNA comprising additional one or more 5′ terminal G nucleotides, wherein the additional one or more 5′ terminal G nucleotides are not present in the protospacer sequence or not complementary to one or more nucleotides at the 3′ terminus of the target sequence in a genome. In some embodiments, the off-target editing can be reduced by at least 5%, 10%, 15%, 20%, 25%, 30%, 35%, 40%, 45%, 50%, 55%, 60%, 65%, 70%, 75%, 80%, 85%, 90%, 95%, or 99%.

Provided herein are guide system nucleic acid molecules comprising a secondary structure. For example, the scaffold region of the guide system nucleic acid molecule recognized by the Cas protein may form a secondary structure such as a stem, a hairpin, and/or a loop. Stems or hairpins, described herein, can be about 3-10 nucleotides in length. Loops can be about 6-20 nucleotides in length. Stems may comprise one or more bulges of 1-10 nucleotides in length.

Further provided herein are guide system nucleic acid molecules comprising a target sequence comprising one or more mismatched nucleotide, i.e. the spacer sequence may comprise one or more nucleotides that are not complementary to the target site sequence in the genome. Spacers described herein may harbor various number of mismatches, for example, 1, 2, 3, 4, 5, 6, 7, 8, 9, 10, 11, 12, 13, 14, or 15 mismatches. In some embodiments, a spacer comprises at most 1, 2, 3, 4, or 5 mismatches. In some embodiments, a spacer does not comprise any mismatch as compared to a protospacer sequence at the target site, i.e. the spacer hybridizes with the target sequence at 100%. Spacers described herein may comprise at least 1 to at least 5 mismatched nucleotides. For example, the spacer may comprise at least 1, at least 2, at least 3, at least 4, or at least 5 mismatched nucleotides. In some embodiments, the spacer may comprise at most 3, at most 4, at most 5, at most 6, or at most 7 mismatched nucleotides. In some embodiments, the one or more mismatched nucleotides may be located at the 5′ terminus of the spacer sequence. In some embodiments, the one or more mismatched nucleotides may be located at the 3′ terminus of the spacer sequence. In some embodiments, the one or more mismatched nucleotides may be internally located in the spacer sequence.

Provided herein are guide system nucleic acid molecules comprising a sequence extension. A sequence extension can be on the 5′ or 3′ terminus or can be added internally. For example, the 5′ terminus of the gRNA Cas12a/Cpf1 (e.g., crRNA) can be extended by 2-59 nucleotides. Extending the 5′ terminus of the Cas12a/Cpf1 gRNA, which comprises a scaffold sequence for Cas12a/Cpf1 binding on the 5′ terminus and a target sequence on the 3′ terminus, can increase the editing efficiency and delivery of Cas12a/Cpf1 in vitro and in vivo. It can also increase tolerance of gRNAs to chemical modifications, leading to enhanced stability of gRNAs. In another example, a gRNA of Cas9 comprising an internal extension of 2-10 nucleotides to extend the stem region of the stem loop structure can increases gene knockout efficiency in CRISPR-Cas9-mediated genome editing. In some instances, a gRNA may comprise two or more of crRNA sequences and tracrRNA sequence and bind two or more Cas proteins and a target DNA sites at two or more distinct regions in the genome. In some embodiments, the gRNAs described herein may comprise a 5′ sequence extension. In some embodiments, the gRNAs described herein may comprise a 3′ sequence extension. In some embodiments, the gRNAs described herein may comprise an internal sequence extension. In some embodiments, the sequence extension can comprise about at least 1 to at least 70 nucleotides. In some embodiments, the sequence extension can comprise at least 1, at least 5, at least 10, at least 20, at least 30, at least 40, at least 50, at least 60, or at least 70 nucleotides.

Provided herein are guide system nucleic acid molecules comprising a nucleotide analog, e.g., guanine (G) analog. A nucleotide analog comprises an alteration in a phosphate backbone, a sugar, and/or nucleobases. Non-limiting examples of nucleotide analog include 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, 2′-fluororibose, ribose, 2′-deoxyribose, arabinose, hexose, phosphorothioate linkages, 5′-N-phosphoramidite linkages, intercalated bases, and/or chemically modified bases. Provided herein are gRNAs comprising a 5′ terminal G analog that can exhibit enhanced editing efficiency of a target sequence when in complex with a CRISPR-Cas system compared to a gRNA lacking 5′ terminal G analog.

Further provided herein are nucleic acid molecules comprising 10 to 100 nucleotides in length. In some embodiments, the synthesized nucleic acid molecules comprises 10, 20, 30, 40, 50, 60, 70, 80, 90, or 100 nucleotides in length. In some embodiments, the synthesized nucleic acid molecules comprises 10 to 20 nucleotides, 10 to 30 nucleotides, 10 to 40 nucleotides, 10 to 50 nucleotides, 10 to 60 nucleotides, 10 to 70 nucleotides, 10 to 80 nucleotides, 10 to 90 nucleotides, 10 to 100 nucleotides, 20 to 30 nucleotides, 20 to 40 nucleotides, 20 to 50 nucleotides, 20 to 60 nucleotides, 20 to 70 nucleotides, 20 to 80 nucleotides, 20 to 90 nucleotides, 20 to 100 nucleotides, 30 to 40 nucleotides, 30 to 50 nucleotides, 30 to 60 nucleotides, 30 to 70 nucleotides, 30 to 80 nucleotides, 30 to 90 nucleotides, 30 to 100 nucleotides, 40 to 50 nucleotides, 40 to 60 nucleotides, 40 to 70 nucleotides, 40 to 80 nucleotides, 40 to 90 nucleotides, 40 to 100 nucleotides, 50 to 60 nucleotides, 50 to 70 nucleotides, 50 to 80 nucleotides, 50 to 90 nucleotides, 50 to 100 nucleotides, 60 to 70 nucleotides, 60 to 80 nucleotides, 60 to 90 nucleotides, 60 to 100 nucleotides, 70 to 80 nucleotides, 70 to 90 nucleotides, 70 to 100 nucleotides, 80 to 90 nucleotides, 80 to 100 nucleotides, or 90 to 100 nucleotides in length. In some embodiments, the synthesized nucleic acid molecules comprises at least 10, at least 20, at least 30, at least 40, at least 50, at least 60, at least 70, at least 80, or at least 90 nucleotides in length. In some embodiments, the synthesized nucleic acid molecules comprises at most 20, at most 30, at most 40, at most 50, at most 60, at most 70, at most 80, at most 90, or at most 100 nucleotides in length. In some embodiments, the synthesized nucleic acid molecules comprises at least 20 nucleotides in length. In some embodiments, the synthesized nucleic acid molecules comprises at least 80 nucleotides in length. In some embodiments, the synthesized nucleic acid molecules comprises at most 30 nucleotides in length.

The synthesized nucleic acid molecules, as described herein, can comprise one or more modifications. For example, the synthesized nucleic acid molecules can comprise synthetic nucleotide, synthetic nucleotide analog, nucleotide derivatives, and/or modified nucleotides. In some embodiments, the one or more modification can increase stability of the synthesized nucleic acid molecules. In some embodiments, the one or more modification can enhance biological activity of the synthesized nucleic acid molecules. In some embodiments, a modification of internucleotide linkage using phosphorothioate (PS) bond substitutes can be introduced to inhibit exonuclease-mediated degradation of nucleic acid molecules. In some embodiments, the one or more modifications can be made at any location of the synthesized nucleic acid molecule. The synthesized nucleic acid molecule, as described herein, can comprise natural nucleosides (e.g., adenosine, guanosine, cytidine, and uridine), nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), and/or modified phosphate groups (e.g., phosphorothioates and 5′-N-phosphoramidite linkages).

Provided herein are nucleic acid molecule comprising a chemical modification. Chemical modifications as described herein may comprise one or more 5′ modifications selected from the group consisting of 5′ triphosphate, 5′ diphosphate, 5′ monophosphate, and 5′ hydroxyl. In another embodiment, the chemical modification comprises one or more ribose modifications selected from the group consisting of 2′-O-methylation (2′OMe), 2′-O-methoxy-ethyl (2′-MOE), 2′-fluoro (2′F), 2-deoxy-2′-thio, and 2′-azido. In some embodiments, the chemical modification comprises one or more internucleotide linkage modifications selected from the group consisting of phosphorothioate, methylphosphonate, phosphonocarboxylate phosphonothiocarboxylate, boranophosphonate, alkylphosphonate, and alkylphosphonate.

Chemical modifications can further comprise modified nucleotides comprising one or more heterocyclic modifications selected from the group consisting of 2,6-Diaminopurine, 2-Aminopurine, inosine, 2-aminoadenosine, N6-methyladenosine, N6,2′-O-dimethyladenosine, N1-methyladenosine, 2-amino-6-chloropurineriboside, 5-methylcytidine, 5-hydroxymethylcytidine, 8-oxo-7,8-dihydroguanosine, pseudouridine, N4-acetylcytidine, 5-bromo-uridine, 5-methyluridine, and 5-nitroindole. In some embodiments, the chemical modification comprises, but is not limited to, modified nucleotides comprising one or more 5′ cap modifications selected from the group consisting of GpppG, 7-methylguanylate (m7GpppG), m2,2,7GpppG, and m7-3′-OGpppG (ARCA).

In some instances, the chemical modification comprises modified nucleotides comprising one or more 5′ cap modifications selected from the group consisting of an attachment chemistry (e.g., biotin), a dye, a cell targeting moiety, an active chemistry, and an amino modifier. In some embodiments, the attachment chemistry can comprise biotin. In some embodiments, the dye comprises fluorescein. In some embodiments, the cell targeting moiety comprises digoxigenin. In some embodiments, the active chemistry comprises azides, acrydite, thiols, or alkynes. In some embodiments, the amino modifier comprises aminoallyl.

### 8. Methods of Administration

The compounds described herein can be administered locally in the subject or systemically. In some embodiments, the compounds can be administered, for example, intraperitoneally, intramuscularly, intra-arterially, orally, intravenously, intracranially, intrathecally, intraspinally, intralesionally, intranasally, subcutaneously, intracerebroventricularly, topically, and/or by inhalation. In an example, the compounds are administered intramuscularly, e.g., by intramuscular injection.

In some embodiments, the compound is administered in accordance with an acute regimen. In certain instances, the compound is administered to the subject once. In other instances, the compound is administered at one time point, and administered again at a second time point. In yet other instances, the compound is administered to the subject repeatedly (e.g., once or twice daily) as intermittent doses over a short period of time (e.g., 2 days, 3 days, 4 days, 5 days, 6 days, a week, 2 weeks, 3 weeks, 4 weeks, a month, or more). In some cases, the time between compound administrations is about 1 day, 2 days, 3 days, 4 days, 5 days, 6 days, a week, 2 weeks, 3 weeks, 4 weeks, a month, or more. In other embodiments, the compound is administered continuously or chronically in accordance with a chronic regimen over a desired period of time. For instance, the compound can be administered such that the amount or level of the compound is substantially constant over a selected time period.

Administration of the compound into a subject can be accomplished by methods generally used in the art. The quantity of the compound introduced may take into consideration factors such as sex, age, weight, the types of disease or disorder, stage of the disorder, and the quantity needed to produce the desired result. Generally, for administering the compound for therapeutic purposes, the cells are given at a pharmacologically effective dose. By “pharmacologically effective amount”, “pharmacologically effective dose”, “therapeutically effective dose”, or “therapeutically effective amount” is an amount sufficient to produce the desired physiological effect or amount capable of achieving the desired result, particularly for treating the condition or disease, including reducing or eliminating one or more symptoms or manifestations of the condition or disease.

The compounds described herein may be administered locally by injection into the (e.g., skeletal muscle and/or non-skeletal muscle) tissue and/or organ being targeted, or by administration in proximity to the tissue being targeted. The compounds described herein may be administered systemically (e.g., orally) such that multiple tissues and/or organs are treated and/or affected.

### 9. Pharmaceutical Compositions

The pharmaceutical compositions of the compounds described herein may comprise a pharmaceutically acceptable carrier. In certain aspects, pharmaceutically acceptable carriers are determined in part by the particular composition being administered, as well as by the particular method used to administer the composition. Accordingly, there is a wide variety of suitable formulations of pharmaceutical compositions described herein (see. e.g., REMINGTON'S PHARMACEUTICAL SCIENCES, 18TH ED., Mack Publishing Co., Easton, PA (1990)).

As used herein, “pharmaceutically acceptable carrier” comprises any of standard pharmaceutically accepted carriers known to those of ordinary skill in the art in formulating pharmaceutical compositions. Thus, the compounds, by themselves, such as being present as pharmaceutically acceptable salts, or as conjugates, may be prepared as formulations in pharmaceutically acceptable diluents; for example, saline, phosphate buffer saline (PBS), aqueous ethanol, or solutions of glucose, mannitol, dextran, propylene glycol, oils (e.g., vegetable oils, animal oils, synthetic oils, etc.), microcrystalline cellulose, carboxymethyl cellulose, hydroxylpropyl methyl cellulose, magnesium stearate, calcium phosphate, gelatin, polysorbate 80 or the like, or as solid formulations in appropriate excipients.

The pharmaceutical compositions often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants (e.g., ascorbic acid, sodium metabisulfite, butylated hydroxytoluene, butylated hydroxyanisole, etc.), bacteriostats, chelating agents such as EDTA or glutathione, solutes that render the formulation isotonic, hypotonic or weakly hypertonic with the blood of a recipient, suspending agents, thickening agents, preservatives, flavoring agents, sweetening agents, and coloring compounds as appropriate.

The pharmaceutical compositions described herein are administered in a manner compatible with the dosage formulation, and in such amount as will be therapeutically effective. The quantity to be administered depends on a variety of factors including, e.g., the age, body weight, physical activity, and diet of the individual, the condition or disease to be treated, and the stage or severity of the condition or disease. In certain embodiments, the size of the dose may also be determined by the existence, nature, and extent of any adverse side effects that accompany the administration of a therapeutic agent(s) in a particular individual.

It should be understood, however, that the specific dose level and frequency of dosage for any particular patient may be varied and may depend upon a variety of factors including the activity of the specific compound employed, the metabolic stability and length of action of that compound, the age, body weight, hereditary characteristics, general health, sex, diet, mode and time of administration, rate of excretion, drug combination, the severity of the particular condition, and the host undergoing therapy.

In certain embodiments, the dose of the compound may take the form of solid, semi-solid, lyophilized powder, or liquid dosage forms, such as, for example, tablets, pills, pellets, capsules, powders, solutions, suspensions, emulsions, suppositories, retention enemas, creams, ointments, lotions, gels, aerosols, foams, or the like, preferably in unit dosage forms suitable for simple administration of precise dosages.

As used herein, the term “unit dosage form” refers to physically discrete units suitable as unitary dosages for humans and other mammals, each unit containing a predetermined quantity of a therapeutic agent calculated to produce the desired onset, tolerability, and/or therapeutic effects, in association with a suitable pharmaceutical excipient (e.g., an ampoule). In addition, more concentrated dosage forms may be prepared, from which the more dilute unit dosage forms may then be produced. The more concentrated dosage forms thus will contain substantially more than, e.g., at least 1, at least 2, at least 3, at least 4, at least 5, at least 6, at least 7, at least 8, at least 9, at least 10, or more times the amount of the therapeutic compound.

Methods for preparing such dosage forms are known to those skilled in the art (see, e.g., REMINGTON'S PHARMACEUTICAL SCIENCES, supra). The dosage forms typically include a conventional pharmaceutical carrier or excipient and may additionally include other medicinal agents, carriers, adjuvants, diluents, tissue permeation enhancers, solubilizers, and the like. Appropriate excipients can be tailored to the particular dosage form and route of administration by methods well known in the art (see, e.g., REMINGTON'S PHARMACEUTICAL SCIENCES, supra).

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinvlpyrrolidone, cellulose, water, saline, syrup, methylcellulose, ethylcellulose, hydroxypropylmethylcellulose, and polyacrylic acids such as Carbopols, e.g., Carbopol 941, Carbopol 980, Carbopol 981, etc. The dosage forms can additionally include lubricating agents such as talc, magnesium stearate, and mineral oil: wetting agents; emulsifying agents: suspending agents; preserving agents such as methyl-, ethyl-, and propyl-hydroxy-benzoates (e.g., the parabens); pH adjusting agents such as inorganic and organic acids and bases; sweetening agents; and flavoring agents. The dosage forms may also comprise biodegradable polymer beads, dextran, and cyclodextrin inclusion complexes.

For oral administration, the therapeutically effective dose can be in the form of tablets, capsules, emulsions, suspensions, solutions, syrups, sprays, lozenges, powders, and sustained-release formulations. Suitable excipients for oral administration include pharmaceutical grades of mannitol, lactose, starch, magnesium stearate, sodium saccharine, talcum, cellulose, glucose, gelatin, sucrose, magnesium carbonate, and the like.

The therapeutically effective dose can also be provided in a lyophilized form. Such dosage forms may include a buffer, e.g., bicarbonate, for reconstitution prior to administration, or the buffer may be included in the lyophilized dosage form for reconstitution with, e.g., water. The lyophilized dosage form may further comprise a suitable vasoconstrictor, e.g., epinephrine. The lyophilized dosage form can be provided in a syringe, optionally packaged in combination with the buffer for reconstitution, such that the reconstituted dosage form can be immediately administered to an individual.

In some embodiments, additional compounds or medications can be co-administered to the subject. Such compounds or medications can be co-administered for the purpose of alleviating signs or symptoms of the disease being treated, reducing side effects caused by induction of the immune response, etc. In some embodiments, for example, the 15-PGDH inhibitor and/or PGE2 activators described herein are administered together with a senolytic agent, a compound to enhance PGE2 levels or PGD2 levels, a compound to decrease Atrogin1 levels or activity, a compound to increase signaling through the EP1, EP2, EP3, EP4, DP1, and/or DP2 receptors, and/or any other compound aiming to enhance muscle mass, strength, endurance or function; or the function, health, or any other desired property of the (e.g., skeletal muscle and/or non-skeletal muscle) tissue and/or organ being targeted.

### 10. Kits

Other embodiments of the compositions described herein are kits comprising a 15-PGDH inhibitor and/or PGE2 activator. The kit typically contains containers, which may be formed from a variety of materials such as glass or plastic, and can include for example, bottles, vials, syringes, and test tubes. A label typically accompanies the kit, and includes any writing or recorded material, which may be electronic or computer readable form providing instructions or other information for use of the kit contents.

In some embodiments, the kit comprises one or more reagents for the treatment of injured, damaged, aged, or diseased (e.g., skeletal muscle and/or non-skeletal muscle) tissue and/or organs. In some embodiments, the kit comprises one or more reagents for the treatment of a (e.g., skeletal muscle and/or non-skeletal muscle) tissue and/or organs in a subject with an age-related condition, disorder, or disease. In some embodiments, the kit comprises an agent that antagonizes the expression or activity of 15-PGDH. In some embodiments, the kit comprises an inhibitory nucleic acid (e.g., a morpholino oligomer, an antisense DNA or RNA, small interfering RNA (siRNA), microRNA (miRNA), short hairpin RNA (shRNA)), or a polynucleotide encoding a 15-PGDH inhibiting polypeptide, that inhibits or suppresses 15-PGDH mRNA or protein expression or activity, e.g., enzyme activity. In some embodiments, the kit comprises a modified RNA, e.g., a modified shRNA or siRNA, or a modified mRNA encoding a polypeptide 15-PGDH inhibitor and/or PGE2 activator. In some embodiments, the kit further comprises one or more plasmid, bacterial or viral vectors for expression of the inhibitory nucleic acid or polynucleotide encoding a 15-PGDH-inhibiting polypeptide. In some embodiments, the kit comprises a peptide nucleic acid (PNA), a morpholino oligomer, an aptamer, antisense oligonucleotide capable of hybridizing to a portion of a 15-PGDH-encoding mRNA. In some embodiments, the kit comprises an antibody (e.g., a monoclonal, polyclonal, humanized, bispecific, chimeric, blocking or neutralizing antibody) or antibody-binding fragment thereof that specifically binds to and inhibits a 15-PGDH protein. In some embodiments, the kit comprises a blocking peptide. In some embodiments, the kit comprises an aptamer (e.g., a peptide or nucleic acid aptamer). In some embodiments, the kit comprises an affimer. In some embodiments, the kit comprises a modified RNA. In particular embodiments, the kit comprises a combination with a small molecule inhibitor, e.g., SW033291, that binds to 15-PGDH or inhibits its enzymatic activity. In some embodiments, the kit further comprises one or more additional therapeutic agents, e.g., agents for administering in combination therapy with the agent that antagonizes the expression or activity of 15-PGDH and/or enhances the expression or activity of PGE2.

In some embodiments, the kits can further comprise instructional materials containing directions (e.g., protocols) for the practice of the methods described herein (e.g., instructions for using the kit for enhancing the function, health, or other properties of (e.g., skeletal muscle and/or non-skeletal muscle) tissues and/or organs). While the instructional materials typically comprise written or printed materials they are not limited to such. Any medium capable of storing such instructions and communicating them to an end user is contemplated by this disclosure. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The present disclosure will be described in greater detail by way of a specific example. The following example is offered for illustrative purposes only, and is not intended to limit the disclosure in any manner. Those of skill in the art will readily recognize a variety of noncritical parameters which can be changed or modified to yield essentially the same results.

### Example 1. Targeting Prostaglandin E2 Degrading Enzyme to Ameliorate Skeletal Muscle and/or Non-Skeletal Muscle Tissue Function in Age-Related Diseases and Conditions

As individuals age, quality of life is reduced and mortality is increased. Age-related diseases are a group of diseases that occur more frequently in people as they age which directly correlate to decreased longevity (1). These age-related diseases include cardiovascular diseases (atrial fibrillation, stroke, ischemic heart diseases, cardiomyopathies, endocarditis, intracerebral hemorrhage), chronic respiratory diseases (chronic obstructive pulmonary disease, asbestosis, silicosis), nutritional diseases (trachoma, diarrheal diseases, encephalitis), kidney diseases (chronic kidney diseases), gastrointestinal and digestive diseases (NASH, pancreatitis, ulcer, intestinal obstruction), neurological disorders (Alzheimer's, dementia, Parkinson's), sensory disorders (hearing loss, macular degeneration, glaucoma), skin and subcutaneous diseases (cellulitis, ulcer, fungal skin diseases, pyoderma), osteoporosis, osteoarthritis, rheumatoid arthritis and the like (2).

Usually, PGE2 may stimulate muscle stem cells (MuSCs) to regenerate damaged muscles in young mice (3), in good agreement with findings regarding its function in regeneration in other tissues, including bone, colon, liver, and blood (4-6). In some embodiments, PGE2 signaling may go awry in aging. In some embodiments, a previously unrecognized role for the PGE2 degrading enzyme, 15-hydroxyprostaglandin dehydrogenase (15-PGDH), in aged tissues has been demonstrated. Partial inhibition of 15-PGDH restores PGE2 and/or PGD2 to youthful levels, and can thereby rejuvenate tissue function. Our findings provide fresh insights into aging and uncover an innovative treatment strategy.

A reduction in PGE2 may be due to increased degradation by 15-PGDH in aged tissues. In some cases, the specific activity of the enzyme may be increased in aged tissues, including cardiac, skin, spleen and colon. Accordingly, inhibition of 15-PGDH can help ameliorate age-related diseases and conditions by restoring or increasing PGE2 and/or PGD2 levels in aged tissues.

In some instances, 15-PGDH may be used as a new marker of aging, detectable at elevated activity in numerous tissues such as heart, skin, colon, and spleen. Restoring PGE2 and/or PGD2 to youthful levels can therefore provide pleiotropic ameliorative effects, as 15-PGDH is upregulated in a range of tissues with aging.

### REFERENCES

1. D. S. Kehler, Age-related disease burden as a measure of population ageing. Lancet Public Health 4, e123-e124 (2019).
2. A. Y. Chang, V. F. Skirbekk, S. Tyrovolas, N. J. Kassebaum, J. L. Dieleman, Measuring population ageing: an analysis of the Global Burden of Disease Study 2017. Lancet Public Health 4, e159-e167 (2019).
3. A. T. V. Ho et al., Prostaglandin E2 is essential for efficacious skeletal muscle stem-cell function, augmenting regeneration and strength. Proc Natl Acad Sci USA 114, 6675-6684 (2017).
4. H. Chen et al., Prostaglandin E2 mediates sensory nerve regulation of bone homeostasis. Nat Commun 10, 181 (2019).
5. T. E. North et al., Prostaglandin E2 regulates vertebrate haematopoietic stem cell homeostasis. Nature 447, 1007-1011 (2007).
6. Y. Zhang et al., Inhibition of the prostaglandin-degrading enzyme 15-PGDH potentiates tissue regeneration. Science 348, aaa2340 (2015).

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A method of regenerating and/or rejuvenating a function of an injured, damaged, aged, or diseased tissue or an injured, damaged, aged, or diseased organ in an individual, the method comprising: administering to the individual a 15-hydroxyprostaglandin dehydrogenase (15-PGDH) inhibitor and/or a prostaglandin E2 (PGE2) activator in an amount effective to inhibit 15-PGDH expression or activity and/or increase PGE2 synthesis or activity in the individual, thereby regenerating and/or rejuvenating the function of the injured, damaged, aged, or diseased tissue or the injured, damaged, aged, or diseased organ in the individual, wherein the 15-PGDH inhibitor and/or PGE2 activator is selected from the group consisting of: a nucleic acid molecule or polypeptide capable of inhibiting 15-PGDH expression, a nucleic acid molecule or polypeptide capable of enhancing PGE2 synthesis, a gene editing system capable of inhibiting 15-PGDH expression, a gene editing system capable of enhancing PGE2 synthesis, an epigenetic modification capable of inhibiting 15-PGDH expression, an epigenetic modification capable of enhancing PGE2 synthesis, and combinations thereof.

2. The method of claim 1, wherein, after the administering, the function of the injured, damaged, aged, or diseased tissue or the injured, damaged, aged, or diseased organ is regenerated and/or rejuvenated relative to a function of the injured, damaged, aged, or diseased tissue or the injured, damaged, aged, or diseased organ prior to the administering the 15-PGDH inhibitor and/or PGE2 activator.

3. The method of claim 1 or 2, wherein, after the administering, the function of the injured, damaged, aged, or diseased tissue or the injured, damaged, aged, or diseased organ is regenerated and/or rejuvenated by at least about 5% relative to a function of the injured, damaged, aged, or diseased tissue or the injured, damaged, aged, or diseased organ prior to the administering the 15-PGDH inhibitor and/or PGE2 activator.

4. The method of any one of claims 1-3, wherein, after the administering, the function of the injured, damaged, aged, or diseased tissue or the injured, damaged, aged, or diseased organ is regenerated and/or rejuvenated to a level substantially similar to a level of a function of a young tissue or a young organ.

5. The method of any one of claims 1-4, wherein, after the administering, the function of the injured, damaged, aged, or diseased tissue or the injured, damaged, aged, or diseased organ is regenerated and/or rejuvenated to a level within at least about 25% of a level of a function of a young tissue or a young organ.

6. The method of any one of claims 1-5, wherein, after the administering, a level of prostaglandin E2 (PGE2) in the injured, damaged, aged, or diseased tissue or the injured, damaged, aged, or diseased organ is increased relative to a level of PGE2 in the injured, damaged, aged, or diseased tissue or the injured, damaged, aged, or diseased organ prior to the administering.

7. The method of any one of claims 1-6, wherein, after the administering, a level of PGE2 in the injured, damaged, aged, or diseased tissue or the injured, damaged, aged, or diseased organ is increased by at least about 5% relative to a level of PGE2 in the injured, damaged, aged, or diseased tissue or the injured, damaged, aged, or diseased organ prior to the administering.

8. The method of any one of claims 1-7, wherein, after the administering, a level of PGE2 in the injured, damaged, aged, or diseased tissue or the injured, damaged, aged, or diseased organ is increased to a level substantially similar to a level of PGE2 present in a young tissue or a young organ.

9. The method of any one of claims 1-8, wherein, after the administering, a level of PGE2 in the injured, damaged, aged, or diseased tissue or the injured, damaged, aged, or diseased organ is increased to a level within at least about 25% of a level of PGE2 present in a young tissue or a young organ.

10. The method of any one of claims 1-9, wherein the administering increases systemic levels of PGE2 in the individual.

11. The method of any one of claims 1-10, wherein the administering results in a regeneration or rejuvenation of serum cytokines to levels substantially similar to serum cytokine levels found in a young individual.

12. The method of claim 11, wherein the serum cytokines are selected from the group consisting of: interleukin-10 (IL10), interleukin-6 (IL6), betacellulin (BTC), granulocyte-macrophage colony-stimulating factor (GM-CSF), interleukin-13 (IL13), tumor necrosis factor alpha (TNF-α), interleukin-1 beta (IL1b), interleukin-22 (IL22), and any combination thereof.

13. The method of any one of claims 1-12, wherein the administering results in: a decrease in serum IL10 levels relative to serum IL10 levels prior to the administering, a decrease in serum IL6 levels relative to serum IL6 levels prior to the administering, a decrease in serum BTC levels relative to serum BTC levels prior to the administering, a decrease in serum GM-CSF levels relative to serum GM-CSF levels prior to the administering, a decrease in serum IL13 levels relative to serum IL13 levels prior to the administering, an increase in serum TNF-alpha levels relative to serum TNF-alpha levels prior to the administering, an increase in serum IL beta levels relative to serum IL1 beta levels prior to the administering, an increase in serum IL22 levels relative to serum IL22 levels prior to the administering, or any combination thereof.

14. The method of any one of claims 1-13, wherein the injured, damaged, aged, or diseased tissue or the injured, damaged, aged, or diseased organ is selected from the group consisting of: skeletal muscle tissue, epidermal tissue, epithelial tissue, vascular tissue, cardiac muscle, brain, bone, cartilage, sensory organs, kidney, thyroid, lung, smooth muscle, brown fat, spleen, liver, heart, small intestine, colon, skin, ovaries and other reproductive tissues, hair, dental tissue, blood, cochlea, and any combination thereof.

15. The method of any one of claims 1-14, wherein the individual has one or more biomarkers of aging, tissue or organ injury, tissue or organ damage, or tissue or organ disease.

16. The method of claim 15, wherein the one or more biomarkers of aging, tissue or organ injury, tissue or organ damage, or tissue or organ disease is selected from the group consisting of: an increase in 15-PGDH levels relative to a young individual, a decrease in PGE2 levels relative to a young individual, an increase in a PGE2 metabolite relative to a young individual, an increase or a greater accumulation of senescent cells relative to a young individual, an increase in expression of one or more atrogenes relative to a young individual, a decrease in mitochondria biogenesis and/or function relative to a young individual, and an increase in transforming growth factor pathway signaling relative to a young individual.

17. The method of any one of claims 1-16, wherein the 15-PGDH inhibitor and/or PGE2 activator is the nucleic acid molecule capable of inhibiting 15-PGDH or the nucleic acid molecule capable of enhancing PGE2 synthesis.

18. The method of claim 17, wherein the nucleic acid molecule capable of inhibiting 15-PGDH or the nucleic acid molecule capable of enhancing PGE2 synthesis is selected from the group consisting of: a peptide nucleic acid (PNA), an aptamer, an antisense oligonucleotide, a morpholino oligomer, microRNA, siRNA, shRNA, and combinations thereof.

19. The method of any one of claims 1-16, wherein the 15-PGDH inhibitor and/or PGE2 activator is the gene editing system capable of inhibiting 15-PGDH expression or the gene editing system capable of enhancing PGE2 synthesis.

20. The method of claim 19, wherein the gene editing system capable of inhibiting 15-PGDH expression or the gene editing system capable of enhancing PGE2 synthesis is selected from the group consisting of: a CRISPR-Cas system, a zinc-finger nuclease system, a transcription activator-like effector nuclease (TALEN) system, and combinations thereof.

21. The method of claim 20, wherein the gene editing system capable of inhibiting 15-PGDH expression or the gene editing system capable of enhancing PGE2 synthesis comprises the CRISPR-Cas system.

22. The method of claim 20 or 21, wherein the CRISPR-Cas system comprises a Cas endonuclease coupled to a guide RNA (gRNA) targeting at least a portion of a polynucleotide sequence encoding 15-PGDH and/or a prostaglandin synthase.

23. The method of claim 22, wherein the Cas endonuclease is Cas9 endonuclease.

24. The method of any one of claims 1-23, wherein the method comprises using the gene editing system capable of inhibiting 15-PGDH expression to insert a silencer sequence near a polynucleotide sequence encoding 15-PGDH, thereby inhibiting 15-PGDH expression.

25. The method of any one of claims 1-24, wherein the method comprises using the gene editing system capable of enhancing PGE2 synthesis to insert an enhancer sequence near a polynucleotide sequence encoding a prostaglandin synthase, thereby enhancing PGE2 synthesis.

26. The method of any one of claims 1-16, wherein the administering results in inhibition of transcription factors that negatively regulate the enhancer or promoter of the HPGD gene.

27. The method of any one of claims 1-16, wherein the administering results in upregulation of transcription factors that enhance an enhancer or promoter of a prostaglandin synthase gene.

28. The method of claim 27, wherein the prostaglandin synthase gene is selected from the group consisting of: PTGES1, PTGES2, COX1, COX2, and combinations thereof.

29. The method of any one of claims 1-16, wherein the 15-PGDH inhibitor and/or PGE2 activator is the polypeptide capable of inhibiting 15-PGDH expression or the polypeptide capable of enhancing PGE2 synthesis.

30. The method of claim 29, wherein the polypeptide capable of inhibiting 15-PGDH expression or the polypeptide capable of enhancing PGE2 synthesis is selected from the group consisting of: a peptide, an antibody, a nanobody, and combinations thereof.

31. The method of any one of claims 1-30, wherein the individual is a human.

32. The method of any one of claims 1-31, wherein the 15-PGDH inhibitor reduces or blocks 15-PGDH expression.

33. The method of any one of claims 1-32, wherein the 15-PGDH inhibitor reduces or blocks enzymatic activity of 15-PGDH.

34. The method of any one of claims 1-33, wherein the PGE2 activator increases expression of a prostaglandin synthase.

35. The method of any one of claims 1-34, wherein the PGE2 activator increases expression of an activating factor of a prostaglandin synthase.

36. The method of any one of claims 1-35, wherein the administering results in decreased levels of a PGE2 metabolite in the injured, damaged, aged, or diseased tissue or the injured, damaged, aged, or diseased organ relative to the injured, damaged, aged, or diseased tissue or the injured, damaged, aged, or diseased organ prior to the administering of the 15-PGDH inhibitor and/or PGE2 activator and/or to a level that is substantially similar to a level present in young tissue or a young organ.

37. The method of claim 36, wherein the PGE2 metabolite is selected from the group consisting of: 15-keto PGE2, 13,14-dihydro-15-keto PGE2, and both.
